(19) United States
(12) Patent Application Publication
(10) Pub. No.: US 2026/0027279 A1
(43) Pub. Date: Jan. 29, 2026

(54) AUTOMATED CLOT ASPIRATION AND BLOOD REINFUSION SYSTEMS, AND ASSOCIATED DEVICES AND METHODS

(71) Applicant: Inari Medical, Inc., Irvine, CA (US)

(72) Inventors: Benjamin Edward Merritt, San Clemente, CA (US); Brian Edward Merritt, San Clemente, CA (US); Donald Joseph Fuller, San Clemente, CA (US); Parker Ozenne, Aliso Viejo, CA (US); Heidi Lynne Silk, Newport Beach, CA (US); Daniel Aaron Leitner, Orange, CA (US); Jakub Marek Truty, San Clemente, CA (US); Jared Shimizu, Tustin, CA (US)

(21) Appl. No.: 19/277,188

(22) Filed: Jul. 22, 2025

Related U.S. Application Data
(60) Provisional application No. 63/675,087, filed on Jul. 24, 2024.
(60) Provisional application No. 63/697,985, filed on Sep. 23, 2024.
(60) Provisional application No. 63/763,445, filed on Feb. 26, 2025.

(51) Int. Cl. A61M 1/36; A61M 1/00; A61M 3/02; A61M 5/20; A61M 39/22; A61M 39/24

(52) U.S. Cl. CPC A61M 1/362264; A61M 1/3613; A61M 1/362265; A61M 1/65; A61M 1/67; A61M 3/0262; A61M 5/2053; A61M 39/24; A61M 2039/226; A61M 2039/229

(57) ABSTRACT

Disclosed herein are devices, systems, and methods for at least partially automatically aspirating clot material and blood from the vasculature of a patient, filtering the blood from the clot material, and returning the blood to the vasculature of the patient. In some embodiments, a system in accordance with the present technology can include (i) an aspiration catheter configured to be positioned within the vasculature of a patient proximate to clot material therein, (ii) an aspiration assembly selectively fluidly couplable to the aspiration catheter, (iii) a filter assembly selectively fluidly couplable to the aspiration assembly, (iv) a reinfusion and flushing assembly selectively fluidly couplable to the filter assembly, (v) a reinfusion catheter selectively fluidly couplable to the reinfusion and flushing assembly and configured to be positioned within the vasculature, and (vi) a control system communicatively coupled to the aspiration assembly, the filter assembly, and/or the reinfusion and flushing assembly.

### CROSS-REFERENCE TO RELATED APPLICATIONS

This application claims the benefit of U.S. Provisional Patent Application No. 63/675,087, filed Jul. 24, 2024, and titled “AUTOMATED CLOT ASPIRATION AND BLOOD RETURN SYSTEMS, AND ASSOCIATED DEVICES AND METHODS,” U.S. Provisional Patent Application No. 63/697,985, filed Sep. 23, 2024, and titled “AUTOMATED CLOT ASPIRATION AND BLOOD REINFUSION SYSTEMS, AND ASSOCIATED DEVICES AND METHODS,” and U.S. Provisional Patent Application No. 63/763,445, filed Feb. 26, 2025, and titled “AUTOMATED CLOT ASPIRATION AND BLOOD REINFUSION SYSTEMS, AND ASSOCIATED DEVICES AND METHODS,” each of which is incorporated herein by reference in its entirety.

### TECHNICAL FIELD

The present technology generally relates to systems, devices, and methods for least partially automatically (i) aspirating clot material and blood from the vasculature of a patient, (ii) filtering the blood from the clot material, and (iii) returning the filtered blood to the vasculature of the patient, and associated devices and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by occlusive material, such as clot material, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

In the pulmonary circulation, occlusive material can cause harm by obstructing pulmonary arteries—a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

Various systems exist for performing thrombectomies or removing occlusive material to reestablish blood flow within a patient. Some such systems can utilize aspiration to remove occlusive material and blood from the patient and can include components for filtering the occlusive material from the blood and returning the filtered blood to the patient. However, such systems are often cumbersome and/or difficult to operate for a surgeon and/or other surgical team members.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially-schematic side cross-sectional view of a clot treatment system in accordance with embodiments of the present technology.

FIG. 2A is a partially-schematic side view of an aspiration catheter and a reinfusion catheter of the system of FIG. 1 with the reinfusion catheter configured as an introducer in accordance with embodiments of the present technology.

FIG. 2B is a partially-schematic side view of a multi-lumen catheter configured as both the aspiration catheter and the reinfusion catheter of FIG. 1 in accordance with embodiments of the present technology.

FIGS. 3A-3N are partially-schematic side cross-sectional views of the clot treatment system of FIG. 1 illustrating a method or sequence performed by the system of aspirating clot material and blood from a patient, filtering the blood from the clot material, reinfusing the filtered blood into the patient, flushing the reinfusion catheter, and flushing a filter assembly of the system in accordance with embodiments with the present technology.

FIGS. 4A-4E are partially-schematic side cross-sectional views of the clot treatment system of FIG. 1 illustrating a method or sequence performed by the system of aspirating clot material and blood from a patient, filtering the blood from the clot material, reinfusing the filtered blood into the patient, flushing the filter assembly, and/or flushing the reinfusion catheter in accordance with additional embodiments with the present technology.

FIGS. 5A-5E are partially-schematic side cross-sectional views of the clot treatment system of FIG. 1 illustrating a method or sequence performed by the system of aspirating clot material and blood from a patient, filtering the blood from the clot material, reinfusing the filtered blood into the patient, flushing the filter assembly, and/or flushing the reinfusion catheter in accordance with additional embodiments with the present technology.

FIGS. 6A-6D are partially-schematic side cross-sectional views of the clot treatment system of FIG. 1 illustrating a method or sequence performed by the system of aspirating clot material and blood from a patient, filtering the blood from the clot material, reinfusing the filtered blood into the patient, flushing the filter assembly, and/or flushing the reinfusion catheter in accordance with additional embodiments with the present technology.

FIGS. 7A-7H are partially-schematic side cross-sectional views of the clot treatment system of FIG. 1 illustrating a method or sequence performed by the system for priming the system to have an initial configuration shown in FIG. 3A in accordance with embodiments of the present technology.

FIG. 8 is a partially-schematic side cross-sectional view of a clot treatment system in accordance with additional embodiments of the present technology.

FIG. 9 is a partially-schematic side cross-sectional view of a clot treatment system in accordance with additional embodiments of the present technology.

FIGS. 10A-10D are a first isometric view, a first side view, a second side view, and a second isometric view, respectively, of a clot treatment system in accordance with additional embodiments of the present technology.

FIG. 11 is a side view of an aspiration syringe of the system of FIGS. 10A-10D in accordance with embodiments of the present technology.

FIGS. 12A-12D are simplified side cross-sectional views of the clot treatment system of FIGS. 10A-10D illustrating a method or sequence performed by the system of aspirating clot material and blood from a patient, filtering the blood from the clot material, and reinfusing the filtered blood into the patient in accordance with embodiments with the present technology.

FIG. 13 is a partially-schematic side view of an aspiration syringe in accordance with additional embodiments of the present technology.

FIG. 14 is a side view of an automated stopcock valve in accordance with embodiments of the present technology.

FIG. 15 is a perspective view of an automated stopcock valve in accordance with additional embodiments of the present technology.

FIG. 16 is a perspective view of an automated syringe in accordance with embodiments of the present technology.

FIG. 17 is a perspective view of a clot treatment system including a pair of the automated syringes of FIG. 16 in accordance with embodiments of the present technology.

FIGS. 18A and 18B are a partially-schematic side view and a side-cross-sectional view, respectively, of a pneumatic syringe in accordance with embodiments of the present technology.

### DETAILED DESCRIPTION

The present technology is generally directed to systems, devices, and methods for at least partially automatically aspirating clot material and blood from the vasculature of a patient, filtering the blood from the clot material, returning the blood to the vasculature of the patient, and/or flushing the system, and associated devices and methods. In some embodiments, a system in accordance with the present technology can include (i) an aspiration catheter configured to be positioned within the vasculature of a patient proximate to clot material within the vasculature, (ii) an aspiration assembly selectively fluidly couplable to the aspiration catheter, (iii) a filter assembly selectively fluidly couplable to the aspiration assembly, (iv) a reinfusion and flushing assembly selectively fluidly couplable to the filter assembly, (v) a reinfusion catheter selectively fluidly couplable to the reinfusion and flushing assembly and configured to be positioned within the vasculature of the patient, and (vi) a control system communicatively coupled to the aspiration assembly, the filter assembly, and/or the reinfusion and flushing assembly.

The control system can be configured to control the aspiration assembly, the filter assembly, and/or the reinfusion and flushing assembly to carry out a myriad of aspiration, filtered blood reinfusion, and system flushing operations during a clot removal procedure (e.g., thrombectomy procedure) carried out on a patient. For example, the control system can control the aspiration assembly to (i) generate vacuum pressure (e.g., negative pressure) and apply the vacuum pressure to the aspiration catheter to aspirate clot material and blood from the vasculature of the patient into the aspiration assembly and (ii) generate positive pressure to drive the aspirated clot material and blood from the aspiration assembly into the filter assembly. The filter assembly is configured to filter the clot material from the blood. The control system can control the reinfusion and flushing assembly to (i) generate vacuum pressure to draw filtered blood from the filter assembly into the reinfusion and flushing assembly and (ii) generate positive pressure to drive the filtered blood from the reinfusion and flushing assembly into the reinfusion catheter to reinfuse the filtered blood into the vasculature. The control system can further control the reinfusion and flushing assembly to drive a flushing fluid into the filter assembly to, for example, flush a filter positioned therein. The control system can further control the reinfusion and flushing assembly to drive the flushing fluid into the reinfusion catheter to push filtered blood therethrough. In some aspects of the present technology, the system enables an automated system for clot removal and blood reinfusion that reduces blood loss for the patient, reduces complication and complexity for an operator (e.g., surgeon, surgical team member), reduces procedure times, increases procedure efficacy, and/or the like.

More particularly, the aspiration assembly can comprise an aspiration inlet tube fluidly coupled to an aspiration lumen of the aspiration catheter, an aspiration outlet tube, aspiration connection tubing, an aspiration syringe having a plunger slidable within a barrel and an aspiration syringe actuator configured to slide the plunger through the barrel, an aspiration inlet valve positioned between the aspiration connection tubing and the aspiration inlet tube, an aspiration syringe valve positioned between the aspiration connection tubing and the aspiration syringe, and an aspiration outlet valve positioned between the aspiration connection tubing and the aspiration outlet tube. The filter assembly can comprise a filter inlet fluidly coupled to the aspiration outlet tube, a filter conduit, a filter between the filter inlet and the filter conduit, and a filter valve. The reinfusion and flushing assembly can comprise reinfusion connection tubing fluidly coupled to the filter conduit via the filter valve, a reinfusion outlet tube fluidly coupled to the reinfusion lumen of the reinfusion catheter, a reinfusion syringe having a plunger slidable within a barrel and a reinfusion syringe actuator configured to slide the plunger through the barrel, a flushing syringe having a plunger slidable within a barrel and a flushing syringe actuator configured to slide the plunger through the barrel, a reinfusion outlet valve positioned between the reinfusion connection tubing and the reinfusion outlet tube, a reinfusion syringe valve positioned between the reinfusion connection tubing and the reinfusion syringe, and a flushing valve positioned between the reinfusion connection tubing and the flushing syringe. The control system can control movement of the various valves and syringes to achieve the clot removal, blood reinfusion, and system flushing operations described herein.

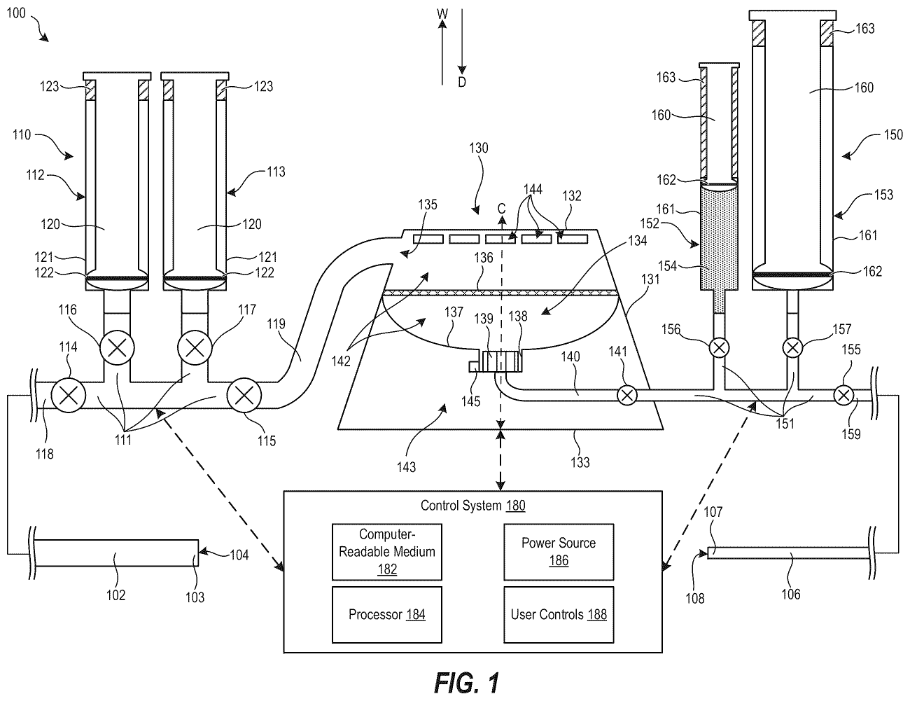

Certain details are set forth in the following description and in FIGS. 1-17 to provide a thorough understanding of various embodiments of the present technology. In other instances, well-known structures, materials, operations, and/or systems often associated with intravascular procedures, clot removal procedures, clot treatment systems, clot treatment devices, fluid control devices, electromechanical actuators, syringes, catheters, blood filtering devices, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, aspiration assembly 110 is first introduced and discussed with reference to FIG. 1.

The headings provided herein are for convenience only and should not be construed as limiting the subject matter disclosed. To the extent any materials incorporated herein by reference conflict with the present disclosure, the present disclosure controls.

### I. SELECTED EMBODIMENTS OF CLOT TREATMENT SYSTEMS

FIG. 1 is a partially-schematic side cross-sectional view of a clot treatment system 100 (“system 100”) in accordance with embodiments of the present technology. The system 100 can also be referred to as an aspiration assembly, a vascular access system, a clot removal system, a thrombectomy system, an aspiration thrombectomy and blood reinfusion system, an automated aspiration thrombectomy and blood reinfusion system, and/or the like. In the illustrated embodiment, the system 100 includes (i) an aspiration catheter 102 configured to be positioned within the vasculature of patient proximate to clot material within the vasculature, (ii) an aspiration assembly 110 selectively fluidly couplable to the aspiration catheter 102, (iii) a filter assembly 130 selectively fluidly couplable to the aspiration assembly 110, (iv) a reinfusion and flushing assembly 150 selectively fluidly couplable to the filter assembly 130, (v) a reinfusion catheter 106 selectively fluidly couplable to the reinfusion and flushing assembly 150 and configured to be positioned within the vasculature of the patient, and (vi) a control system 180 (e.g., a computing device, an electronics system, an electronics subsystem, a control system, control assembly, a controller, and/or the like) communicatively coupled to the aspiration assembly 110, the filter assembly 130, and/or the reinfusion and flushing assembly 150.

In general, the control system 180 is configured to control the aspiration assembly 110 to (i) generate vacuum pressure (e.g., negative pressure) and apply the vacuum pressure to the aspiration catheter 102 to aspirate clot material and blood from the vasculature of the patient into the aspiration assembly 110 and (ii) generate positive pressure to drive/force the aspirated clot material and blood from the aspiration assembly 110 into the filter assembly 130. The filter assembly 130 is configured to filter the clot material from the blood and to permit the filtered blood to pass from the filter assembly 130 to the reinfusion and flushing assembly 150 while the clot material remains in the filter assembly 130. The control system 180 is configured to control the reinfusion and flushing assembly 150 to (i) generate vacuum pressure to draw the filtered blood from the filter assembly 130 into the reinfusion and flushing assembly 150 and (ii) generate positive pressure to drive the filtered blood from the reinfusion and flushing assembly 150 into the reinfusion catheter 106 to reinfuse the filtered blood into the vasculature. The control system 180 can be further configured to control the reinfusion and flushing assembly 150 to drive a flushing fluid into the filter assembly 130 to, for example, flush a filter positioned therein. The control system 180 can further control the reinfusion and flushing assembly 150 to drive the flushing fluid into the reinfusion catheter 106 to drive filtered blood therethrough. The control system 180 is configured to control operation of the aspiration assembly 110, the filter assembly 130, and the reinfusion and flushing assembly 150 to at least partially perform the functions described herein. In some aspects of the present technology, the system 100 enables an automated system for clot removal and blood reinfusion that reduces blood loss for the patient, reduces complication and complexity for an operator (e.g., surgeon, surgical team member), reduces procedure times, increases procedure efficacy, and/or the like.

More specifically, the aspiration catheter 102 can be an elongate member (e.g., a sheath, a shaft) defining an aspiration lumen 104 and configured to be inserted into and through a patient's vasculature and used to, for example, remove or otherwise treat clot material therein. The aspiration catheter 102 can have a distal end portion 103 configured to be positioned proximate to the clot material within the vasculature, such as proximal to, within, and/or distal to the clot material within the vasculature. The aspiration catheter 102 can be a large bore catheter having, for example, a size equal to or greater than 16 French, such as 18 French, 20 French, 22 French, 24 French, 26 French, 28 French, 30 French, 32 French, and/or the like, and a corresponding inner diameter across an inner surface defining the aspiration lumen 104. In some embodiments, the clot material comprises a pulmonary embolism within a pulmonary artery of the patient, a deep vein thrombosis (DVT) within a peripheral vein of the patient, and/or the like. The aspiration catheter 102 can have varying lengths, flexibilities, shapes, thicknesses, and/or other properties along its length. For example, the aspiration catheter 102 can comprise one or more coils, braids, and/or other structures positioned between one or more liner layers (e.g., an inner liner layer and an outer liner layer). In some embodiments, the aspiration catheter 102 can include several features generally similar or identical in structure and/or function to any of the catheters described in (i) U.S. patent application Ser. No. 17/529,018, titled “CATHETERS HAVING SHAPED DISTAL PORTIONS, AND ASSOCIATED SYSTEMS AND METHODS,” and filed Nov. 17, 2021, (ii) U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, (iii) U.S. patent application Ser. No. 18/159,507, titled “ASPIRATION CATHETERS HAVING GROOVED INNER SURFACE, AND ASSOCIATED SYSTEM AND METHODS,” and filed Jan. 25, 2023, and/or (iv) U.S. patent application Ser. No. 18/463,960, titled “CATHETERS HAVING MULTIPLE COIL LAYERS, AND ASSOCIATED SYSTEMS AND METHODS,” and filed Sep. 8, 2023, each of which is incorporated by reference herein in its entirety.

In the illustrated embodiment, the aspiration assembly 110 comprises aspiration connection tubing 111, an aspiration inlet tube 118, an aspiration outlet tube 119, a first aspiration syringe 112, a second aspiration syringe 113, an aspiration inlet valve 114, an aspiration outlet valve 115, a first aspiration syringe valve 116, and a second aspiration syringe valve 117. In some embodiments, each of the aspiration inlet valve 114, the aspiration outlet valve 115, the first aspiration syringe valve 116, and the second aspiration syringe valve 117 (collectively valves 114-117) is a valve configured to be controlled by the control system 180 to open and close (e.g., move between an open position and a closed position). For example, the aspiration valves 114-117 can be stopcock valves, solenoid valves, pinch valves, ball valves, and/or the like that can be controlled by the control system 180 to selectively permit/inhibit fluid flow therethrough. In some embodiments, some or all of the aspiration valves 114-117 are stopcocks of any of the types described in U.S. patent application Ser. No. 18/182,966, titled “FLUID CONTROL DEVICES FOR CLOT TREATMENT SYSTEMS, AND ASSOCIATED SYSTEMS AND METHODS,” and filed Aug. 22, 2023, that can be controlled by the control system 180 to open and close. In some embodiments, one or more of the aspiration valves 114-117 comprise a check valve. In some embodiments, the aspiration connection tubing 111 and the aspiration valves 114-117 each have a size and a corresponding inner diameter defining a lumen (e.g., bore) having a size that is the same as or greater than a size of the aspiration catheter 102 and a corresponding inner diameter defining the aspiration lumen 104. For example, the aspiration connection tubing 111 and the aspiration valves 114-117 can each have a size and corresponding inner diameter equal to or greater than about 16 French, about 18 French, about 20 French, about 22 French, about 22 French, about 24 French, and/or the like.

In some embodiments, the first aspiration syringe 112 and the second aspiration syringe 113 are identical or at least generally identical—each including a plunger 120 slidably positioned within a barrel 121. The plunger 120 can include a seal 122 (e.g., an O-ring) positioned to slidably contact and seal against an interior surface of the barrel 121. The barrels 121 of the first and second aspiration syringes 112, 113 can have the same volumes or different volumes. For example, the barrel 121 of the first aspiration syringe 112 and/or the barrel 121 of the second aspiration syringe 113 can have a volume equal to or greater than about 30 cubic centimeters (cc), about 40 cc, about 50 cc, about 60 cc, about 80 cc, about 100 cc, about 150 cc, about 200 cc, and/or the like. In some embodiments, the barrels 121 of the first and second aspiration syringes 112, 113 each have a volume of about 60 cc. In the illustrated embodiment, the first and second aspiration syringes 112, 113 each further include an actuator 123 (e.g., a motor, an electromechanical actuator, a mechanical actuator, a pneumatic actuator, and/or the like) configured to be controlled by the control system 180 to (i) withdraw/retract the plunger 120 through the barrel 121 (e.g., in a direction W) to generate vacuum pressure within the barrel 121 and (ii) depress/advance the plunger 120 through the barrel 121 (e.g., in a direction D) to expel any contents therein. The first and second aspiration syringes 112, 113 are shown in a depressed configuration in FIG. 1 with the plungers 120 fully depressed through the barrels 121 thereof. In some embodiments, the first and second aspiration syringes 112, 113 are large-bore syringes, such as any of those described in detail in U.S. Pat. No. 11,559,382, titled “SYSTEM FOR TREATING EMBOLISM AND ASSOCIATED DEVICES AND METHODS,” and filed Aug. 8, 2019, which is incorporated herein by reference in its entirety.

In the illustrated embodiment, the aspiration inlet valve 114 is movable/actuatable (e.g., via the control system 180) to provide a fluid path therethrough from the aspiration lumen 104 of the aspiration catheter 102 to the aspiration connection tubing 111 via the aspiration inlet tube 118. That is, the aspiration inlet valve 114 can be actuated to move between (i) an open position in which the aspiration connection tubing 111 is fluidly coupled to the aspiration lumen 104 of the aspiration catheter 102 (e.g., fluid is able to flow from the aspiration lumen 104 to the aspiration connection tubing 111) and (ii) a closed position in which the aspiration connection tubing 111 is fluidly decoupled from the aspiration lumen 104 of the aspiration catheter 102 (e.g., fluid is inhibited or even prevented from flowing from the aspiration lumen 104 to the aspiration connection tubing 111). In some embodiments, the aspiration inlet valve 114 is a check valve configured to (i) permit fluid flow therethrough from the aspiration lumen 104 of the aspiration catheter 102 to the aspiration connection tubing 111 via the aspiration inlet tube 118 and (ii) inhibit or even prevent fluid flow therethrough from the aspiration connection tubing 111 to the aspiration lumen 104 of the aspiration catheter 102 via the aspiration inlet tube 118.

In the illustrated embodiment, the aspiration outlet valve 115 is movable/actuatable (e.g., via the control system 180) to provide a fluid path therethrough from the aspiration connection tubing 111 to the filter assembly 130 via the aspiration outlet tube 119. That is, the aspiration outlet valve 115 can be actuated to move between (i) an open position in which the aspiration connection tubing 111 is fluidly coupled to the filter assembly 130 (e.g., fluid is able to flow from the aspiration connection tubing 111 to the filter assembly 130) and (ii) a closed position in which the aspiration connection tubing 111 is fluidly decoupled from the filter assembly 130 (e.g., fluid is inhibited or even prevented from flowing from the aspiration connection tubing 111 to the filter assembly 130). In some embodiments, the aspiration outlet valve 115 is a check valve configured to (i) permit fluid flow therethrough from the aspiration assembly 110 to the filtering assembly 130 and (ii) inhibit or even prevent fluid flow therethrough from the filtering assembly 130 to the aspiration assembly 110.

The first aspiration syringe valve 116 can be movable/actuatable (e.g., via the control system 180) to provide a fluid path therethrough from the first aspiration syringe 112 to the aspiration connection tubing 111. That is, the first aspiration syringe valve 116 can be actuated to move between (i) an open position in which the aspiration connection tubing 111 is fluidly coupled to the first aspiration syringe 112 (e.g., fluid is able to flow from the barrel 121 of the first aspiration syringe 112 to the aspiration connection tubing 111 and from the aspiration connection tubing 111 to the barrel 121 of the first aspiration syringe 112) and (ii) a closed position in which the aspiration connection tubing 111 is fluidly decoupled from the first aspiration syringe 112 (e.g., fluid is inhibited or even prevented from flowing between the aspiration connection tubing 111 and the first aspiration syringe 112).

Similarly, the second aspiration syringe valve 117 can be movable/actuatable (e.g., via the control system 180) to provide a fluid path therethrough from the second aspiration syringe 113 to the aspiration connection tubing 111. That is, the second aspiration syringe valve 117 can be actuated to move between (i) an open position in which the aspiration connection tubing 111 is fluidly coupled to the second aspiration syringe 113 (e.g., fluid is able to flow from the barrel 121 of the second aspiration syringe 113 to the aspiration connection tubing 111 and from the aspiration connection tubing 111 to the barrel 121 of the second aspiration syringe 113) and (ii) a closed position in which the aspiration connection tubing 111 is fluidly decoupled from the second aspiration syringe 113 (e.g., fluid is inhibited or even prevented from flowing between the aspiration connection tubing 111 and the second aspiration syringe 113).

In the illustrated embodiment, the filter assembly 130 includes a body 131 having an upper portion 132 (e.g., a first portion) and a lower portion 133 (e.g., a second portion). The body 131 defines an interior or chamber 134 and an opening or inlet 135 near the upper portion 132 that is fluidly coupled to the aspiration outlet tube 119 of the aspiration assembly 110 for, for example, receiving aspirated blood and clot material therethrough, as described in further detail below. The filter assembly 130 can further include a filter plate or filter tray 136 spanning laterally across the chamber 134 below the inlet 135, and a collection component 137 spanning laterally across the chamber 134 below the filter tray 136. In some embodiments, the collection component 137 slopes downward in a direction toward a central axis C of the filter assembly 130 and includes a lowermost receiving portion 138 configured to receive a filter 139 therein.

A filter conduit 140 (e.g., a filter tube) can be fluidly coupled downstream of the filter 139, and a filter valve 141 can be fluidly coupled along the filter conduit 140. In some embodiments, the filter valve 141 is a valve configured to be controlled by the control system 180 to open and close (e.g., move between an open position and a closed position). For example, the filter valve 141 can be similar or identical to the aspiration valves 114-117 and can comprise a stopcock valve, a solenoid valve, a ball valve, and/or the like that can be controlled by the control system 180 to selectively permit/inhibit fluid flow therethrough. More specifically, the filter valve 141 can be movable/actuatable (e.g., via the control system 180) to provide a fluid path therethrough from the filter conduit 140 to the reinfusion and flushing assembly 150. That is, the filter valve 141 can be actuated to move between (i) an open position in which the filter conduit 140 is fluidly coupled to the reinfusion and flushing assembly 150 (e.g., fluid is able to flow from the filter conduit 140 to the reinfusion and flushing assembly 150) and (ii) a closed position in which the filter conduit 140 is fluidly decoupled from the reinfusion and flushing assembly 150 (e.g., fluid is inhibited or even prevented from flowing from the filter conduit 140 to the reinfusion and flushing assembly 150). In other embodiments, the filter valve 141 can be a check valve that, for example, is configured to (i) permit fluid flow therethrough from the from the filtering assembly 130 to the reinfusion and flushing assembly 150 and (ii) inhibit or even prevent fluid flow therethrough from the reinfusion and flushing assembly 150 to the filtering assembly 130.

In some embodiments, the filter tray 136 has a first porosity and the filter 139 has a second porosity less than the first porosity (e.g., the filter tray 136 has larger pores than the filter 139 to permit larger particles to pass therethrough). For example, the filter tray 136 can have a micron (μm) rating equal to greater than about 500 μm, about 600 μm, about 700 μm, about 800 μm, about 900 μm, about 1000 μm, about 1100 μm, about 2000 μm, about 3000 μm, and/or the like. The filter 139 can have a micron rating equal to or greater than about 10 μm, about 20 μm, about 30 μm, about 40 μm, about 50 μm, about 100 μm, about 200 μm, about 300 μm, about 400 μm, about 500 μm, and/or the like. In some embodiments the filter 139 comprises multiple filter layers having different porosities. For example, the filter 139 can comprise a first filter layer having a first porosity and a second filter layer having second porosity less than the first porosity. The first filter layer can be positioned radially outward relative to the second filter layer. In some embodiments, the first filter layer has a micron rating of about 200 μm and the second filter layer has a micron rating of about 40 μm. In some embodiments, the filter 139 comprises one or more filter layers arranged in a pleated arrangement about the central axis C and positioned to allow fluid (e.g., blood) to pass laterally therethrough from the receiving portion 138 of the collection component 137 to the filter conduit 140. In some embodiments, the filter assembly can have some components generally similar or identical to, and can operate generally similarly or identically, to any of the filter devices described in U.S. patent application Ser. No. 18/963,471, titled “FILTERING DEVICES, SUCH AS FOR USE WITH CLOT TREATMENT SYSTEMS, AND ASSOCIATED SYSTEMS AND METHODS,” and filed Nov. 27, 2024, which is incorporated by reference herein in its entirety.

In operation, the filter assembly 130 can receive blood and clot material into the chamber 134 through the inlet 135 from the aspiration outlet tube 119 of the aspiration assembly 110. The blood and clot material can move (e.g., flow) downward toward/onto/through the filter tray 136 via a gravity. The filter tray 136 can inhibit or even prevent larger portions of the aspirated material (e.g., larger portions of the clot material, coagulated blood) from flowing therethrough while permitting smaller portions of the aspirated material (e.g., blood, smaller portions of the clot material) to move therethrough downward toward/onto the collection component 137. Accordingly, the filter tray 136 provides a first filter stage that filters out large portions of the clot material. The collection component 137 can direct the first-stage filtered material toward the filter 139 positioned within the receiving portion 138. In some embodiments, the reinfusion and flushing assembly 150 can generate negative pressure to draw the first-stage filtered material through the filter 139, through the filter conduit 140, and through the filter valve 141 when the filter valve 141 is open. The filter 139 can provide a second filtering stage that filters out smaller portions of clot material while permitting blood to pass therethrough. Additionally or alternatively, the first-stage filtered material can flow through the filter 139 at least partially due to gravity. Accordingly, the filter assembly 130 is configured to receive aspirated blood and clot material from the aspiration assembly 110, filter the clot material from the blood, and permit the filtered blood to pass to the reinfusion and flushing assembly 150. In some aspects of the present technology, the filter tray 136 can filter out large portions of the clot material that may otherwise clog or interfere with the operation of the filter 139.

In some embodiments, the body 131 is at least partially transparent to allow a user of the system 100 (e.g., a surgeon and/or healthcare team member) to view clot material collected on the filter tray 136 and/or proximate to the filter 139. In the illustrated embodiment, the collection component 137 divides the chamber 134 into an upper chamber portion 142 and a lower chamber portion 143. The upper chamber portion 142 is configured to receive the aspirated blood and clot material and, in some embodiments, can have a volume greater or significantly greater than the volume of the first and second aspiration syringes 112, 113. For example, the upper chamber portion 142 can have a volume equal to or greater than about 100, about 200 cc, about 300 cc, about 400 cc, about 500 cc, about 600 cc, about 1000 cc, and/or the like. Accordingly, the upper chamber portion 142 can be sized to store/hold material aspirated multiple times via the first and second aspiration syringes 112, 113, as described in greater detail below. In some embodiments, the lower chamber portion 143 is substantially sterile and/or isolated from the aspirated material. In some embodiments, some or all of the components of the control system 180 are positioned/housed within the lower chamber portion 143.

In the illustrated embodiment, the body 131 further includes one or more air vents 144 positioned to permit air to vent from the upper chamber portion 142. The filter assembly 130 can optionally include a sensor 145 configured to detect a level of fluid (e.g., blood) within the chamber 134. The control system 180 can be communicatively coupled to the sensor 145 for receiving data about the level of fluid within the chamber 134. The sensor 145 can comprise a pressure sensor, a capacitance-sensor, and/or the like.

The reinfusion and flushing assembly 150 can have several components generally similar or identical to the aspiration assembly 110. For example, in the illustrated embodiment the reinfusion and flushing assembly 150 comprises reinfusion connection tubing 151, a reinfusion outlet tube 159, a flushing syringe 152, a reinfusion syringe 153, a reinfusion outlet valve 155, a flushing syringe valve 156, and a reinfusion syringe valve 157. In some embodiments, each of the reinfusion outlet valve 155, the flushing syringe valve 156, and the reinfusion syringe valve 157 (collectively reinfusion valves 155-157) is an electromechanical valve configured to be controlled by the control system 180 to open and close (e.g., move between an open position and a closed position). For example, the reinfusion valves 155-157 can be stopcock valves, solenoid valves, ball valves, and/or the like that can be controlled by the control system 180 to selectively permit/inhibit fluid flow therethrough. In other embodiments, one or more of the reinfusion valves 155-157 can comprise a check valve. In some embodiments, the reinfusion connection tubing 151 and the reinfusion valves 155-157 each define a lumen (e.g., bore) having a size that is smaller than the corresponding lumens of the aspiration connection tubing 111 and the aspiration valves 114-117 of the aspiration assembly 110.

In some embodiments, the flushing syringe 152 and the reinfusion syringe 153 are identical or at least generally identical to one another and/or to the first and second aspiration syringes 112, 113 of the aspiration assembly 110. For example, in the illustrated embodiment the flushing and reinfusion syringes 152, 153 each include a plunger 160 slidably positioned within a barrel 161. The plunger 160 can include a seal 162 (e.g., an O-ring) positioned to slidably contact and seal against an interior surface of the barrel 161. The barrels 161 of the flushing and reinfusion syringes 152, 153 can have the same volumes or different volumes. For example, in the illustrated embodiment the barrel 161 of the flushing syringe 152 has a volume less than a volume of the reinfusion syringe 153 and less than the volumes of the first and second aspiration syringes 112, 113 of the aspiration assembly 110. Likewise, in the illustrated embodiment the barrel 161 of the reinfusion syringe 153 has a volume greater than the volumes of the first and second aspiration syringes 112, 113. In other embodiments, the volume of the reinfusion syringe 153 can be the same as or about the same as the volumes of the first and second aspiration syringes 112, 113, such as about 60 cc. In some embodiments the barrel 161 of the flushing syringe 152 has a volume equal to greater than about 15 cc, about 30 cc, about 40 cc, about 50 cc, about 60 cc, and/or the like, and the barrel 161 of the reinfusion syringe 153 has a volume equal to or greater than about 60 cc, about 80 cc, about 100 cc, about 150 cc, about 200 cc, about 300 cc, about 500 c, and/or the like. In other embodiments, the various syringes 112, 113, 152, and 153 can have different volumes and/or volumes relative to one another. In the illustrated embodiment, the flushing and reinfusion syringes 152, 153 each further include an actuator 163 (e.g., a motor, an electromechanical actuator, a mechanical actuator, a pneumatic actuator, and/or the like) configured to be controlled by the control system 180 to (i) withdraw/retract the plunger 160 through the barrel 161 (e.g., in the direction W) to generate vacuum pressure within the barrel 161 and/or (ii) depress/advance the plunger 160 through the barrel 161 (e.g., in the direction D) to expel any contents therein. The reinfusion syringe 153 is shown in a depressed configuration in FIG. 1 with the plunger 160 fully depressed through the barrel 161 thereof, while the flushing syringe 152 is shown in a withdrawn configuration with the plunger 160 fully withdrawn through the barrel 161 thereof. In the illustrated embodiment, a flushing fluid 154 (e.g., saline) is positioned within the barrel 161 of the flushing syringe 152.

In the illustrated embodiment, the reinfusion outlet valve 155 is movable/actuatable (e.g., via the control system 180) to provide a fluid path therethrough from the reinfusion connection tubing 151 to the reinfusion catheter 106 via the reinfusion outlet tube 159. That is, the reinfusion outlet valve 155 can be actuated to move between (i) an open position in which the reinfusion connection tubing 151 is fluidly coupled to the reinfusion catheter 106 (e.g., fluid is able to flow from the reinfusion connection tubing 151 to the reinfusion catheter 106) and (ii) a closed position in which the reinfusion connection tubing 151 is fluidly decoupled from the reinfusion catheter 106 (e.g., fluid is inhibited or even prevented from flowing from the reinfusion connection tubing 151 to the reinfusion catheter 106). In some embodiments, the reinfusion outlet valve 155 is a check valve configured to (i) permit fluid flow therethrough from the reinfusion connection tubing 151 to the reinfusion outlet tube 159 and the reinfusion catheter 106 and (ii) inhibit or even prevent fluid flow therethrough from the reinfusion connection tubing 151 to the reinfusion outlet tube 159 and the reinfusion catheter 106.

The flushing syringe valve 156 can be movable/actuatable (e.g., via the control system 180) to provide a fluid path therethrough from the flushing syringe 152 to the reinfusion connection tubing 151. That is, the flushing syringe valve 156 can be actuated to move between (i) an open position in which the reinfusion connection tubing 151 is fluidly coupled to the flushing syringe 152 (e.g., fluid is able to flow from the barrel 161 of the flushing syringe 152 to the reinfusion connection tubing 151) and (ii) a closed position in which the reinfusion connection tubing 151 is fluidly decoupled from the flushing syringe 152 (e.g., fluid is inhibited or even prevented from flowing between the reinfusion connection tubing 151 and the flushing syringe 152).

Similarly, the reinfusion syringe valve 157 can be movable/actuatable (e.g., via the control system 180) to provide a fluid path therethrough from the reinfusion syringe 153 to the reinfusion connection tubing 151. That is, the reinfusion syringe valve 157 can be actuated to move between (i) an open position in which the reinfusion connection tubing 151 is fluidly coupled to the reinfusion syringe 153 (e.g., fluid is able to flow from the barrel 161 of the reinfusion syringe 153 to the reinfusion connection tubing 151 and from the reinfusion connection tubing 151 to the barrel 161 of the reinfusion syringe 153) and (ii) a closed position in which the reinfusion connection tubing 151 is fluidly decoupled from the reinfusion syringe 153 (e.g., fluid is inhibited or even prevented from flowing between the reinfusion connection tubing 151 and the reinfusion syringe 153).

The reinfusion catheter 106 can be an elongate member (e.g., a sheath, a shaft) defining a reinfusion lumen 108 and configured to be inserted into and through the patient's vasculature and used to, for example, reinfuse filtered blood into the vasculature. The reinfusion catheter 106 can have a distal end portion 107 configured to be positioned within the vasculature, and can have a construction the same as or different than the aspiration catheter 102 described in detail above. In some embodiments, the reinfusion catheter 106 can be separate from the aspiration catheter 102 and can be inserted into the patient through a vascular access site different than a vascular access site of the aspiration catheter 102. In some such embodiments, the reinfusion catheter 106 can have a smaller size than the aspiration catheter 102.

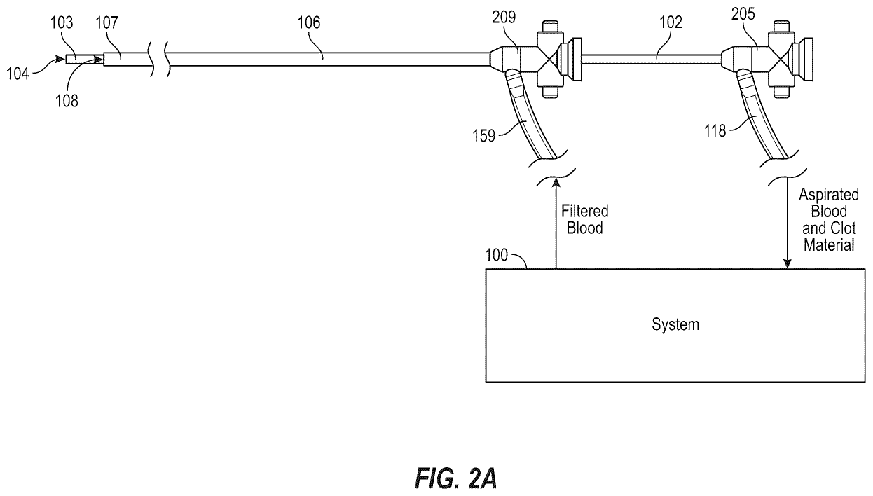

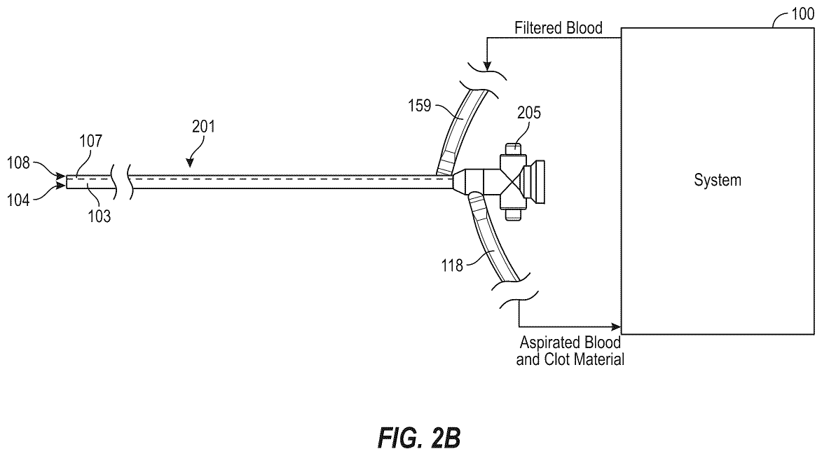

In other embodiments, the reinfusion catheter 106 can serve as introducer catheter (e.g., sheath) for the aspiration catheter 102. For example, FIG. 2A is a partially-schematic side view of the aspiration catheter 102 and the reinfusion catheter 106 of FIG. 1 with the reinfusion catheter 106 configured as an introducer in accordance with embodiments of the present technology. In the illustrated embodiment, a proximal end portion of the aspiration catheter 102 is coupled to an aspiration access valve 205 (e.g., a hemostasis valve) configured to selectively provide access to the aspiration lumen 104 of the aspiration catheter 102, and a proximal end portion of the reinfusion catheter 106 is coupled to a reinfusion access valve 209 (e.g., a hemostasis valve) configured to selectively provide access to the reinfusion lumen 108 of the reinfusion catheter 106. The aspiration and reinfusion access valves 205, 209 can be of the type disclosed in U.S. Pat. No. 11,559,382, titled “HEMOSTASIS VALVES AND METHODS OF USE,” and filed Aug. 30, 2018, which is incorporated herein by reference in its entirety. The aspiration catheter 102 can extend through the reinfusion access valve 209 and through the reinfusion lumen 108 of the aspiration catheter 102 such that, for example, the distal end portion 103 of the aspiration catheter 102 extends distally beyond the distal end portion 107 of the reinfusion catheter 106. In some embodiments, one or more clot treatment devices (e.g., mechanical thrombectomy devices) can be inserted through the aspiration access valve 105 during a clot treatment procedure using the system 100.

A first access tube, such as the aspiration inlet tube 118 of the aspiration assembly 110 of FIG. 1, can fluidly couple the aspiration lumen 104 of the aspiration catheter 102 to the system 100 (e.g., the aspiration assembly 110). A second access tube, such as the reinfusion outlet tube 159 of the reinfusion and flushing assembly 150 of FIG. 1, can fluidly couple the reinfusion lumen 108 of the reinfusion catheter 106 to the system 100 (e.g., the reinfusion and flushing assembly 150). As described in further detail below, the system 100 can aspirate blood and clot material through the aspiration lumen 104 of the aspiration catheter 102, filter the blood from the clot material, and reinfuse/return the filtered blood through the reinfusion lumen 108 of the reinfusion catheter 106. In some aspects of the present technology, configuring the reinfusion catheter 106 as an introducer sheath/catheter for the aspiration catheter 102 can reduce the number of vascular access sites needed for a thrombectomy procedure-improving procedure efficiency and reducing patient discomfort.

In further embodiments, the reinfusion catheter 106 and the aspiration catheter 102 can comprise different lumens of the same multi-lumen. For example, FIG. 2B is a partially-schematic side view of a multi-lumen (e.g., dual-lumen) catheter 201 serving as both the aspiration catheter 102 and the reinfusion catheter 106 of FIG. 1 in accordance with embodiments of the present technology. In the illustrated embodiment, the aspiration lumen 104 extends at least partially parallel to the reinfusion lumen 108, and the distal end portion 103 of the aspiration lumen 104 terminates at the same location as the distal end portion 107 of the reinfusion lumen 108. In some embodiments, the aspiration lumen 104 has a greater cross-sectional dimension (e.g., area, diameter, radius) than a corresponding cross-sectional dimension of the reinfusion lumen 108. As described in greater detail below, in some aspects of the present technology the system 100 can aspirate blood and clot material and reinfuse filtered blood at different volumetric rates-enabling the aspiration lumen 104 to have a larger dimension than the reinfusion lumen 108 to, for example, maximize aspiration forces. In some embodiments, the multi-lumen catheter 201 can be of the type described in U.S. patent application Ser. No. 18/885,201, titled “MULTI-LUMEN ASPIRATION CATHETERS, AND ASSOCIATED SYSTEMS AND METHODS,” and filed Sep. 13, 2024, which is incorporated by reference herein in its entirety. In some embodiments, a proximal end portion of the multi-lumen catheter 201 is coupled to the access valve 205 configured to selectively provide access to the aspiration lumen 104.

A first access tube, such as the aspiration inlet tube 118 of the aspiration assembly 110 of FIG. 1, can fluidly couple the aspiration lumen 104 to the system 100 (e.g., the aspiration assembly 110). A second access tube, such as the reinfusion outlet tube 159 of the reinfusion and flushing assembly 150 of FIG. 1, can fluidly couple the reinfusion lumen 108 to the system 100 (e.g., the reinfusion and flushing assembly 150). As described in further detail below, the system 100 can aspirate blood and clot material through the aspiration lumen 104, filter the blood from the clot material, and reinfuse/return the filtered blood through the reinfusion lumen 108. In some aspects of the present technology, integrating the reinfusion catheter 106 and the aspiration catheter 102 can reduce the number of vascular access sites needed for a thrombectomy procedure as well as reducing the overall profile needed for the access site-improving procedure efficiency and reducing patient discomfort.

Referring to FIGS. 1-2B, in some aspects of the present technology the aspiration assembly 110 can operate independently from the reinfusion and flushing assembly 150 to aspirate clot material and blood and pass the clot material and blood to the filter assembly 130 for storage. Accordingly, the rate at which the aspiration assembly 110 aspirates material is independent from the rate at which the reinfusion and flushing assembly 150 reinfuses filtered blood into the patient. That is, the rate at which fluid is removed from the patient need not be the same as the rate at which fluid is reintroduced into the patient. Accordingly, a size of the aspiration catheter 102 need not be the same as a size of the reinfusion catheter 106. This enables the reinfusion catheter 106 to utilize a smaller access site if introduced separately from the aspiration catheter 102 into the patient, or to comprise an introducer sheath for the aspiration catheter (FIG. 2A), or smaller lumen of a multi-lumen catheter (FIG. 2B).

Referring again to FIG. 1, the control system 180 can include a non-transitory computer-readable medium 182, a processor 184, a power source 186, and one or more user controls 188. The computer-readable medium 182 can store instructions that, when executed by the processor 184, carry out the functions attributed to the control system 180 as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

The power source 186 can provide power to the processor 184, to the various valves (e.g., the aspiration valves 114-117, the filter valve 141, the reinfusion valves 155-157) to operate (e.g., open and close) the valves, to the actuators 123, 163 to operate the syringes 112, 113, 152, 153, and/or to other electrical components of the system 100. The power source 186 can comprise one or more batteries, a connection to a source of electrical power in a hospital (e.g., within an operating room), and/or the like. As described above, some or all of the non-transitory computer-readable medium 182, the processor 184, and the power source 186 can be located at least partially within the lower chamber portion 143 of the filter assembly 130.

The user controls 188 can comprise one or more buttons, sliders, touchscreen elements, and/or the like that can be actuated, triggered, selected, and/or the like by a user of the system 100 to control one or more operations of the system 100. The user controls 188 can be utilized by the user to start/stop some or all of the aspiration, filtering, reinfusion, and/or filtering operations described in detail herein. The user controls 188 can be operably coupled to the processor 184 via a wired or wireless connection. In some embodiments, user controls 188 comprise one or more buttons positioned on the filter assembly 130 (e.g., on an external surface of the body 131), on the aspiration catheter 102 (e.g., on a proximal portion thereof), and/or elsewhere in/on the system 100. For example, the one or more buttons can comprise a first button that is actuatable to start/stop aspiration via the aspiration assembly 110 and a second button configured to start/stop reinfusion and/or flushing via the reinfusion and flushing assembly 150. In some embodiments, the user controls 188 can be omitted and the system 100 can operate entirely automatically.

In some embodiments, one or more of the syringes, valves, etc., can be operated manually by an operator (e.g., a surgeon, a surgical team member) rather than via the control system 180. Likewise, the control system 180 can be configured to control one or more aspects of the system 100 via pneumatic, mechanical, and/or other non-electronic means. For example, one or more of the first and second aspiration syringes 112, 113, the flushing syringe 152, and/or the reinfusion syringe 153 can comprise a pneumatic syringe and the control system 180 can include a switch, trigger, and/or the like that is actuatable to cause the syringe(s) to withdraw/depress to generate negative/positive pressure. In some embodiments, the control system 180 can include a pneumatic switch, trigger, and/or actuator of any of the type described in, for example, U.S. Provisional Patent Application No. 63/763,433, titled “PNEUMATIC SYRINGES, SUCH AS FOR CLOT ASPIRATION, AND ASSOCIATED SYSTEMS AND METHODS,” and filed Feb. 26, 2025, which is incorporated herein by reference in its entirety. The pneumatic switch, trigger, and/or actuator may, with a single trigger press, sequentially: operate the first and/or second aspiration syringes 112, 113 to generate negative pressure to aspirate clot and blood and then operate the first and/or second aspiration syringes 112, 113 to generate positive pressure to drive the clot and blood into the filter assembly 130.

In some embodiments, each of the first and second aspiration syringes 112, 113 can be removably coupled to the aspiration connection tubing 111 of the aspiration assembly 110 such that they can be decoupled for cleaning, replacement, etc. Likewise, the flushing syringe 152 and the reinfusion syringe 153 can be removably coupled to the reinfusion connection tubing 151 of the reinfusion and flushing assembly 150 to permit cleaning, replacement, refilling, etc. For example, the flushing syringe 152 can be decoupled from the reinfusion connection tubing 151 to permit refilling of the barrel 161 with the flushing fluid 154.

In some embodiments, each of the electronic and/or electronically-controlled components of the system 100, such as the control system 180, the various valves (e.g., the aspiration valves 114-117, the filter valve 141, the reinfusion valves 155-157), and the actuators 123, 163, can be decoupled from the system 100. Accordingly, such components can be reused in multiple clot treatment procedures, while the tubing components, 111, 118, 119, 140, 151, and 159, the syringes 112, 113, 152, 153, the filter assembly 130, etc., can be disposable.

### II. SELECTED EMBODIMENTS OF OPERATING CLOT TREATMENT SYSTEMS

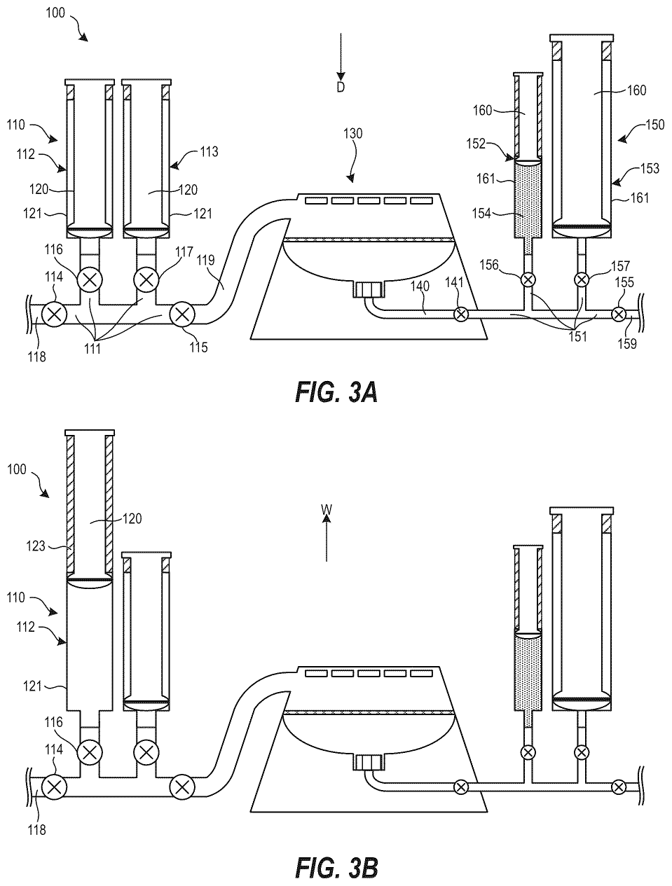

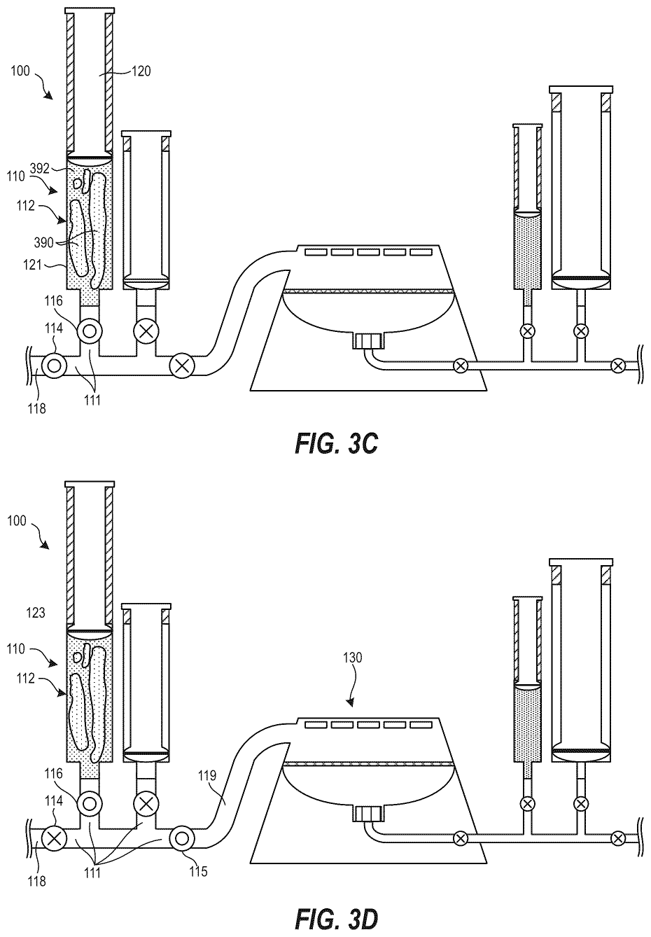

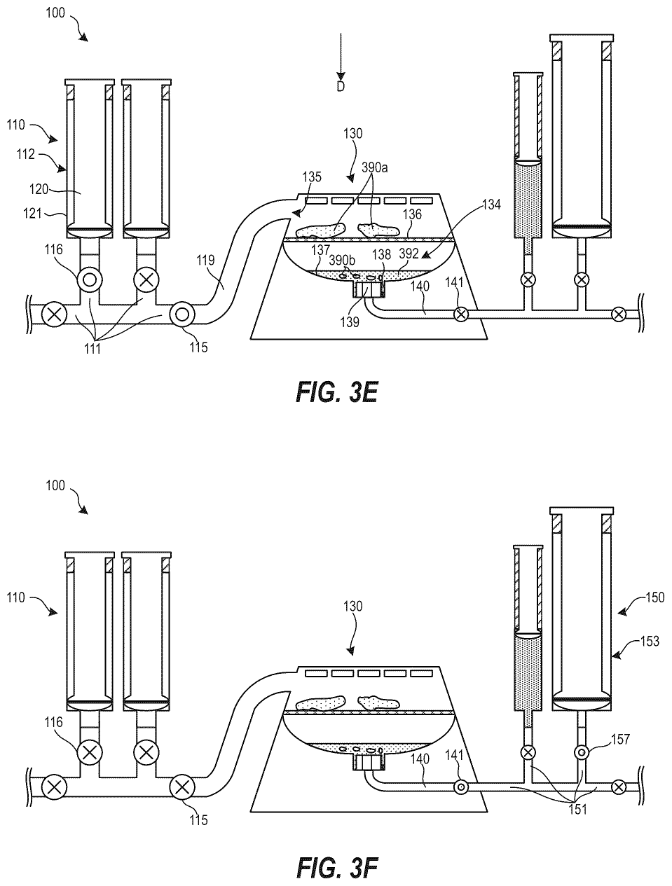

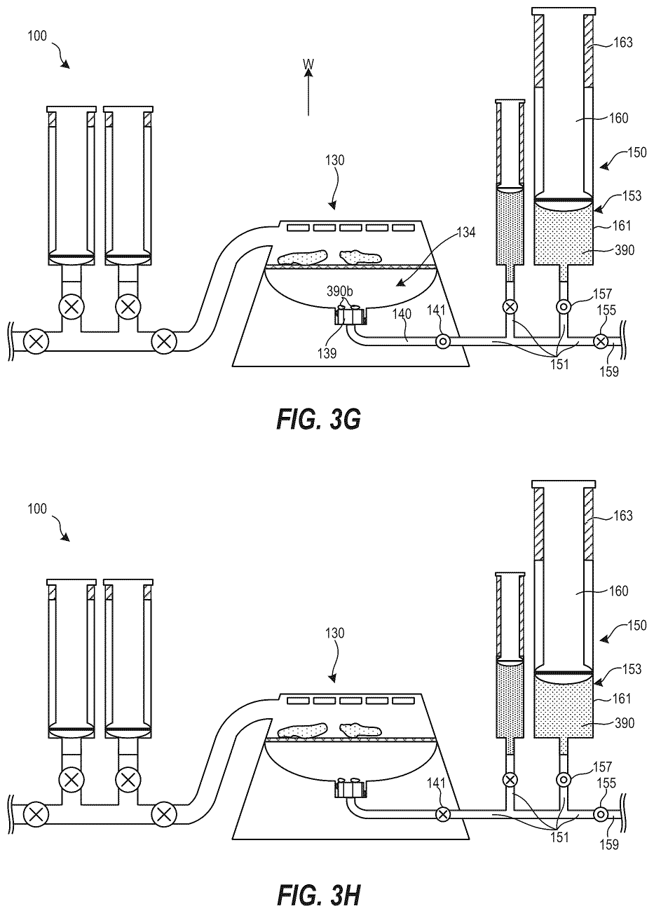

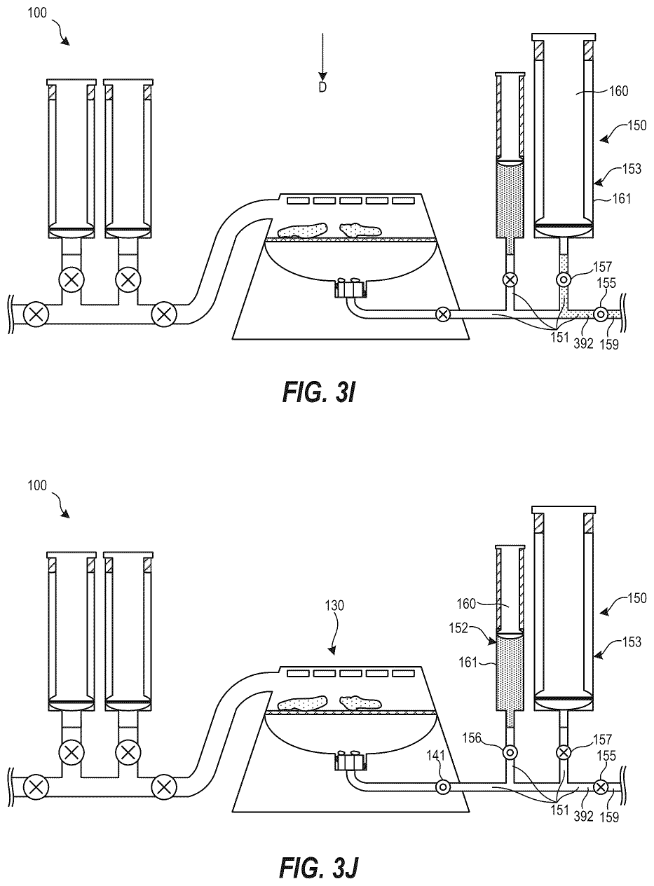

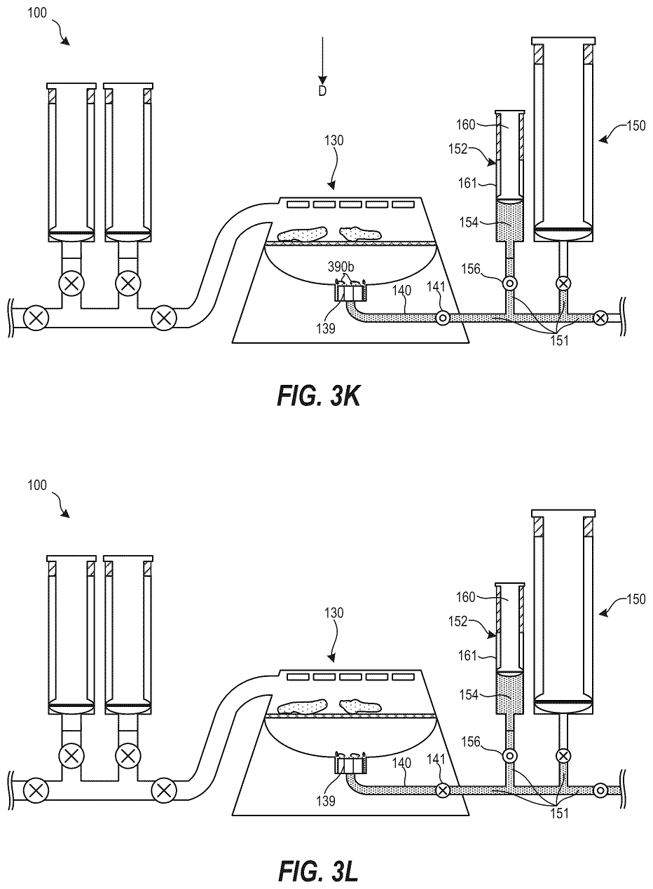

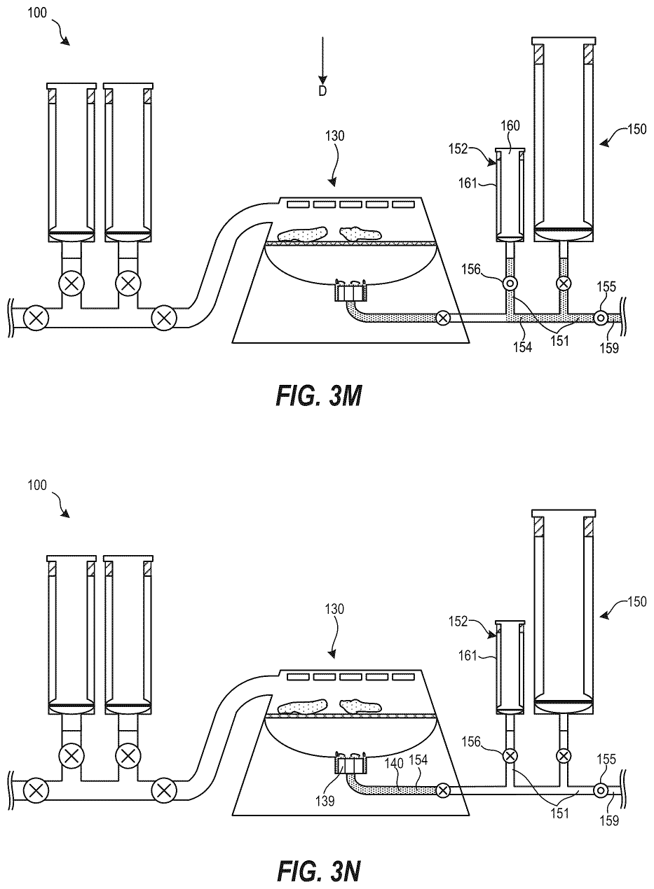

The control system 180 can control the various valves (e.g., the aspiration valves 114-117, the filter valve 141, the reinfusion valves 155-157) and the actuators 123, 163 to operate the syringes 112, 113, 152, 153 in various sequences to perform a myriad of aspiration, filtered blood reinfusion, and system flushing operations during a clot removal procedure (e.g., thrombectomy procedure) carried out on a patient. Some such operations are described in detail below with reference to FIGS. 3A-7H. However, one of ordinary skill in the art will appreciate that the various operations may be combined and/or modified. Moreover, while the various valves (e.g., aspiration valves 114-117, the filter valve 141, and the reinfusion valves 155-157) are described as electromechanically controlled in FIGS. 3A-7H, in other embodiments one or more of the valves can comprise a check valve or other type of valve that passively opens and closes to selectively provide for fluid flow in one direction and inhibit fluid flow in the other direction.

FIGS. 3A-3N are partially-schematic side cross-sectional views of the clot treatment system 100 of FIG. 1 illustrating a method or sequence performed by the system 100 of aspirating clot material and blood from a patient, filtering the blood from the clot material, reinfusing the filtered blood into the patient, flushing the reinfusion catheter 106, and flushing the filter assembly 130 (e.g., to prepare the system 100 for another aspiration and/or filtering step) in accordance with embodiments with the present technology. Although the aspiration catheter 102 and the reinfusion catheter 106 are not shown in FIGS. 3A-3N for clarity, with additional reference to FIG. 1, the distal end portion 103 of the aspiration catheter 102 can be positioned proximate to clot material within the vasculature of a patient, such as proximal to, within, and/or distal to the clot material within the vasculature, and the aspiration inlet tube 118 of the aspiration assembly 110 can be fluidly coupled to the aspiration lumen 104 of the aspiration catheter 102. In some embodiments, the clot material comprises a pulmonary embolism within a pulmonary artery of the patient, a deep vein thrombosis (DVT) within a peripheral vein of the patient, and/or the like. The reinfusion catheter 106 can likewise be positioned within the vasculature of the patient through the same or a different access site as the aspiration catheter, as described in detail above with reference to FIGS. 1-2B, and the reinfusion outlet tube 159 can be fluidly coupled to the reinfusion lumen 108 of the reinfusion catheter 106.

Referring to FIG. 3A, in an initial configuration the various tubing components 111, 118, 119, 140, 151, and 159 of the aspiration, filter, and reinfusion and flushing assemblies 110, 130, 150 can be flushed with a flushing fluid (e.g., saline), and each of the aspiration valves 114-117, the filter valve 141, and the reinfusion valves 155-157 can then be moved to a closed state (e.g., as indicated by an “X” on each valve) by the control system 180 (FIG. 1). In the initial configuration, the first aspiration syringe 112, the second aspiration syringe 113, and the reinfusion syringe 153 can each be in a depressed state (e.g., with the plungers 120 of the first and second aspiration syringes 112, 113 fully depressed in the direction D through the barrels 121 thereof, and the plunger 160 of the reinfusion syringe 153 fully depressed in the direction D through the barrel 161 thereof). The flushing syringe 152 can be in a withdrawn configuration with the plunger 160 fully withdrawn through the barrel 161 thereof and the flushing fluid 154 (e.g., saline) at least partially filling the barrel 161 of the flushing syringe 152.

Referring to FIG. 3B, the control system 180 (FIG. 1) can then actuate the actuator 123 of the first aspiration syringe 112 to withdraw the plunger 120 in the direction W through the barrel 121 of the first aspiration syringe 112 to generate vacuum pressure (e.g., negative pressure) within the barrel 121. Because the first aspiration syringe valve 116 is closed, vacuum is charged within the barrel 121 of the first aspiration syringe 112 (e.g., a negative pressure is maintained) before the first aspiration syringe 112 is fluidly connected to the aspiration lumen 104 of the aspiration catheter 102 (FIG. 1). In some embodiments, the aspiration inlet valve 114 can be opened before withdrawing the plunger 120 of the first aspiration syringe 112.

Referring to FIG. 3C, the control system 180 (FIG. 1) can then open the first aspiration syringe valve 116 and the aspiration inlet valve 114 (e.g., as indicated by an “0” on each valve) to fluidly connect the barrel 121 of the first aspiration syringe 112 to the aspiration lumen 104 of the aspiration catheter 102 (FIG. 1) via the aspiration inlet tube 118 and the aspiration connection tubing 111. The first aspiration syringe valve 116 can be opened at the same time as the aspiration inlet valve 114, before the aspiration inlet valve 114 is opened, or after the aspiration inlet valve 114 is opened. For example, in some embodiments the aspiration inlet valve 114 is opened before withdrawing the plunger 120 of the first aspiration syringe 112. Accordingly, the vacuum stored in the barrel 121 of the first aspiration syringe 112 is applied to the aspiration lumen 104 of the aspiration catheter 102 (FIG. 1) to aspirate clot material 390 and blood 392 through the aspiration lumen 104 of the aspiration catheter 102, through the aspiration inlet tube 118, through the aspiration inlet valve 114, through the aspiration connection tubing 111, through the first aspiration syringe valve 116, and into the barrel 121 of the first aspiration syringe 112. In some embodiments, opening the first aspiration syringe valve 116 instantaneously or nearly instantaneously applies the stored vacuum pressure to the aspiration lumen 104 of the aspiration catheter 102, thereby generating a suction pulse throughout the aspiration lumen 104 that can aspirate the clot material 390 and the blood 392 into and through the aspiration lumen 104. In some aspects of the present technology, such aspiration via stored vacuum pressure can have a multitude of benefits as described in, for example, U.S. Pat. No. 11,559,382, titled “SYSTEM FOR TREATING EMBOLISM AND ASSOCIATED DEVICES AND METHODS,” and filed Aug. 8, 2019, which is incorporated herein by reference in its entirety. In other embodiments, the first aspiration syringe valve 116 and the aspiration inlet valve 114 can be opened before the plunger 120 of the first aspiration syringe 112 is withdrawn to generate vacuum pressure such that the clot material 390 is aspirated continuously as the vacuum pressure is generated—that is, for example, without or substantially without storing the vacuum pressure in the barrel 121 of the first aspiration syringe 112.

In some aspects of the present technology, the first aspiration syringe 112 is no longer under vacuum after opening the aspiration syringe valve 116. Accordingly, the blood 392 is not held under vacuum therein, which can inhibit or even prevent damage to the blood 392—for example, as compared to systems that store blood under vacuum such that it may boil or otherwise damagingly increase in temperature due to the reduced boiling point caused by the reduced pressure.

Referring to FIG. 3D, the control system 180 (FIG. 1) can then (i) close the aspiration inlet valve 114 to fluidly disconnect the first aspiration syringe 112 from the aspiration inlet tube 118 and the aspiration catheter 102 (FIG. 1) and (ii) open the aspiration outlet valve 115 to fluidly connect the first aspiration syringe 112 to the filter assembly 130 via the aspiration connection tubing 111 and the aspiration outlet tube 119.

Referring to FIG. 3E, the control system 180 (FIG. 1) can then actuate the actuator 123 of the first aspiration syringe 112 to depress the plunger 120 through the barrel 121 of the first aspiration syringe 112 to drive the clot material 390 (identified as larger portions of clot material 390a and smaller portions of clot material 390b) and the blood 392 through the first aspiration syringe valve 116, through the aspiration connection tubing 111, through the aspiration outlet tube 119, and into the chamber 134 of the filter assembly 130. The clot material 390 and the blood 392 are received through the inlet 135 of the filter assembly 130, and gravity causes the clot material 390 and the blood 392 to move downward through the chamber 134 toward the filter tray 136 and the collection component 137. As described in detail above with reference to FIG. 1, the filter tray 136 can filter larger portions 390a of the clot material 390 while allowing the blood 392 and smaller portions 390b of the clot material 390 to pass therethrough. The blood 392 and the smaller portions 390b of the clot material 390a can collect on the collection component 137 and/or in the receiving portion 138 thereof around the filter 139.

Referring to FIG. 3F, the control system 180 (FIG. 1) can then (i) open the filter valve 141 to fluidly connect the filter conduit 140 to the reinfusion connection tubing 151 and (ii) open the reinfusion syringe valve 157 to fluidly connect the reinfusion syringe 113 to the filter assembly 130 via the reinfusion connection tubing 151. The control system 180 can also optionally close the first aspiration syringe valve 116 and the aspiration outlet valve 115 to fluidly disconnect the filter assembly 130 from the aspiration assembly 110.

Referring to FIG. 3G, the control system 180 (FIG. 1) can then actuate the actuator 163 of the reinfusion syringe 153 to withdraw the plunger 160 through the barrel 161 of the reinfusion syringe 153 in the direction W to generate vacuum force to pull the blood 392 from the filter assembly 130 into the barrel 161 of the reinfusion syringe 153. More specifically, the reinfusion syringe 153 can pull the blood 392 through the filter 139, through the filter conduit 140, through the filter valve 141, through the reinfusion connection tubing 151, and through the reinfusion syringe valve 157 into the barrel 161 of the reinfusion syringe 153. As described in detail above with reference to FIG. 1, the filter 139 filters out the smaller portions 390b of the clot material 390 from the blood 392 such that the blood 392 is suitable for reinfusion into the patient. In some embodiments, where the reinfusion syringe 153 is larger than the first aspiration syringe 112, the plunger 160 of the reinfusion syringe 153 can be withdrawn only partially to receive all of the filtered blood 392. In other embodiments, such as where the reinfusion syringe 153 and the first aspiration syringe 112 are the same or generally similar sizes, the plunger 160 of the reinfusion syringe 153 can be fully or further withdrawn to receive all of the filtered blood 392.

In some embodiments, the reinfusion syringe 153 is sized to inhibit or even prevent drawing a greater volume than a volume of the blood 392 in the filter assembly 130 to inhibit or even prevent air being drawn into the reinfusion syringe 153, which could subsequently form one or more air emboli if injected into the patient. Alternatively or additionally, the control system 180 can receive information from the sensor 145 (FIG. 1) about a level of the blood 392 within the chamber 134 and automatically end retraction of the plunger 160 of the reinfusion syringe 153 when the level of the blood 392 in the filter assembly 130 is below a predetermined threshold. In this manner, the system 100 can inhibit or even prevent the reinfusion syringe 153 from drawing air into the barrel 161 thereof.

Referring to FIG. 3H, the control system 180 (FIG. 1) can then (i) close the filter valve 141 to fluidly disconnect the filter assembly 130 from the reinfusion and flushing assembly 150 and (ii) open the reinfusion outlet valve 155 to fluidly connect the barrel 161 of the reinfusion syringe 153 to the reinfusion lumen 108 of the reinfusion catheter 106 (FIG. 1) via the reinfusion outlet tube 159 and the reinfusion connection tubing 151.

Referring to FIG. 3I, the control system 180 (FIG. 1) can then actuate the actuator 163 of the reinfusion syringe 153 to depress the plunger 160 through the barrel 161 of the reinfusion syringe 153 to drive the filtered blood 392 through the reinfusion syringe valve 157, through the reinfusion connection tubing 151, through the reinfusion outlet valve 155, through the reinfusion outlet tube 159, and into and at least partially through the reinfusion lumen 108 of the reinfusion catheter 106 (FIG. 1). At least some of the filtered blood 392 can exit the reinfusion lumen 108 into the vasculature of the patient.

Referring to FIG. 3J, the control system 180 (FIG. 1) can then (i) open the filter valve 141 to fluidly connect the filter assembly 130 to the reinfusion and flushing assembly 150, (ii) open the flushing syringe valve 156 to fluidly connect the barrel 161 of the flushing syringe 152 to the filter assembly 130 via the filter conduit 140 and the reinfusion connection tubing 151, (iii) close the reinfusion syringe valve 157 to fluidly disconnect the reinfusion syringe 153 from the reinfusion connection tubing 151, and (iv) close the reinfusion outlet valve to fluidly disconnect the reinfusion and flushing assembly 150 from the reinfusion catheter 106 (FIG. 1).

Referring to FIG. 3K, the control system 180 (FIG. 1) can then actuate the actuator 163 of the flushing syringe 152 to at least partially depress the plunger 160 in the direction D through the barrel 161 of the flushing syringe 152 to drive the flushing fluid 154 through the flushing syringe valve 156, through the reinfusion connection tubing 151, through the filter valve 141, through the filter conduit 140, and into and at least partially through the filter 139. The flushing fluid 154 can clear/flush the filter 139 of the smaller portions 390b of the clot material 390 and/or from any residual blood thereon.

Referring to FIG. 3L, the control system 180 (FIG. 1) can then (i) close the filter valve 141 to fluidly disconnect the filter assembly 130 from the reinfusion and flushing assembly 150 and (ii) open the reinfusion outlet valve 155 to fluidly connect the reinfusion and flushing assembly 150 to the reinfusion catheter 106 (FIG. 1).

Referring to FIG. 3M, the control system 180 (FIG. 1) can then actuate the actuator 163 of the flushing syringe 152 to further depress the plunger 160 in the direction D through the barrel 161 of the flushing syringe 152 to drive the flushing fluid 154 through the flushing syringe valve 156, through the reinfusion connection tubing 151, through the reinfusion outlet valve 155, through the reinfusion outlet tube 159, and into and at least partially through the reinfusion lumen 108 of the reinfusion catheter 106 (FIG. 1). The flushing fluid 154 can drive/force/push any of the filtered blood 392 (FIG. 3I) remaining in the reinfusion lumen 108 of the reinfusion catheter 106 out of the reinfusion lumen 108 and into the vasculature of the patient.

Referring to FIG. 3N, the control system 180 (FIG. 1) can then (i) close the flushing syringe valve 156 to fluidly disconnect the barrel 161 of the flushing syringe 152 from the reinfusion connection tubing 151 and (ii) close the reinfusion outlet valve 155 to fluidly disconnect the reinfusion and flushing assembly 150 from the reinfusion catheter 106 (FIG. 1). In some embodiments, a portion of the flushing fluid 154 remains in the filter conduit 140 and/or around the filter 139 of the filter assembly 130.

At this point, the system 100 is configured in the initial configuration shown in FIG. 3A, albeit without flushing fluid filling the barrel 161 of the reinfusion syringe 153. To repeat the same or a different method, the reinfusion syringe 153 can be decoupled from the reinfusion connection tubing 151 of the reinfusion and flushing assembly 150 and refilled with the flushing fluid 154. In other embodiments, the barrel 161 of the flushing syringe 152 can be large enough to hold enough of the flushing fluid to perform multiple operations—that is, with only a portion of the flushing fluid 154 being utilized in a given cycle/method. In some embodiments, flushing of the filter 139 (FIG. 3K) can be performed after driving the flushing fluid 154 through the reinfusion lumen 108 of the reinfusion catheter (FIG. 3M). In some embodiments, the flushing of the filter 139 (FIG. 3K) can be omitted if the subsequent aspiration/filtration steps do not need to be performed.

As noted above, the control system 180 can control the various valves (e.g., the aspiration valves 114-117, the filter valve 141, the reinfusion valves 155-157) and the actuators 123, 163 to operate the syringes 112, 113, 152, 153 in various sequences to perform a myriad of aspiration, filtered blood reinfusion, and system flushing operations during a clot removal procedure (e.g., thrombectomy procedure) carried out on a patient. Likewise, various operations can be combined and/or performed simultaneously. For example, after driving the clot material 390 and the blood 392 to the filter assembly 130 as shown in FIG. 3E, the system 100 can repeat the aspiration steps shown in FIGS. 3B-3D at the same time as the system 100 performs the filtering, flushing, and reinfusing steps shown in FIGS. 3F-3N by closing the aspiration outlet valve 115 to fluidly disconnect and isolate the aspiration assembly 110 from the filter assembly 130 and the reinfusion and flushing assembly 150. Accordingly in some embodiments aspiration and blood filtration and reinfusion can be performed concurrently, nearing a continuous aspiration and reinfusion state.

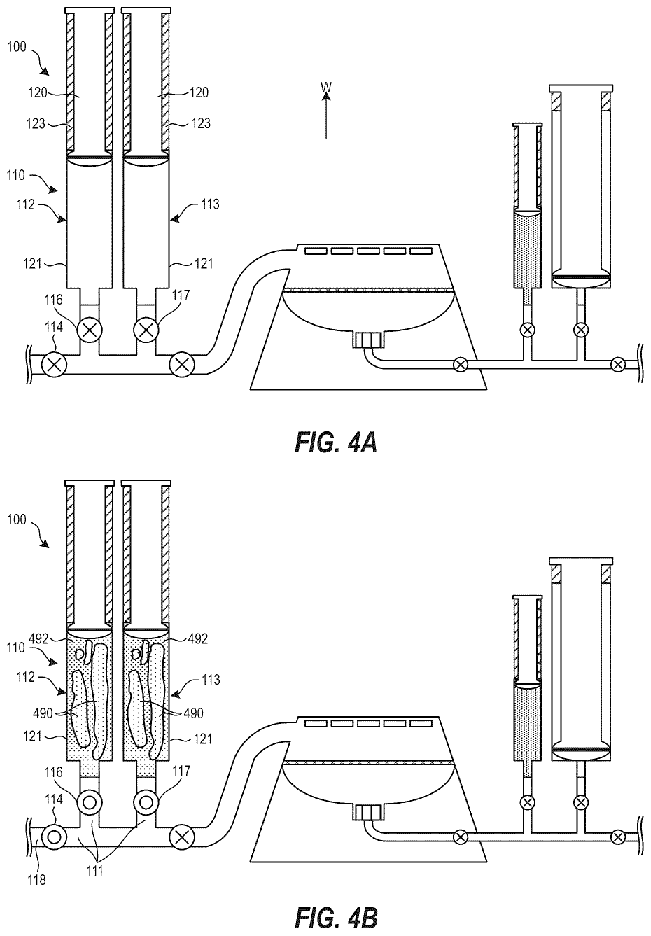

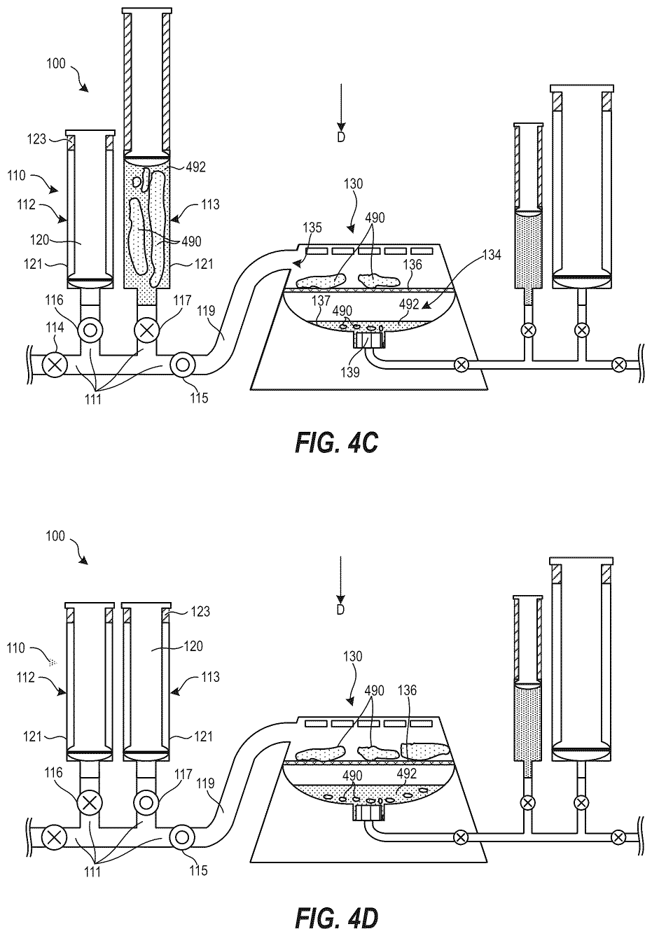

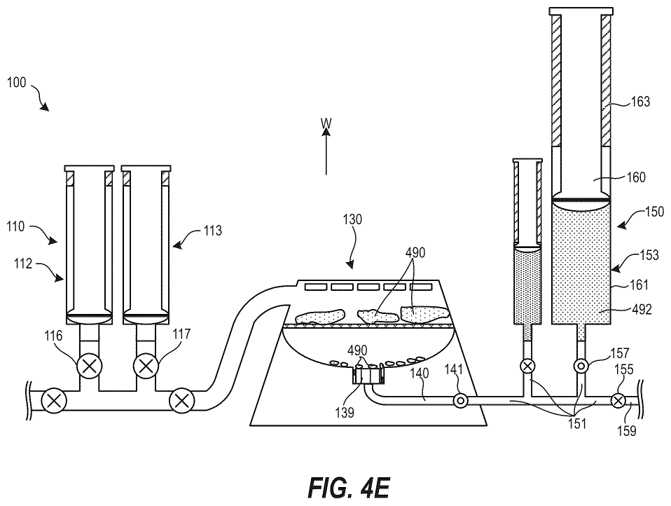

FIGS. 4A-4E are partially-schematic side cross-sectional views of the clot treatment system 100 of FIG. 1 illustrating a method or sequence performed by the system 100 of aspirating clot material and blood from a patient, filtering the blood from the clot material, reinfusing the filtered blood into the patient, flushing the filter assembly 130, and/or flushing the reinfusion catheter 106 in accordance with additional embodiments with the present technology. The system 100 can initially have the initial configuration shown in FIG. 3A. Then, referring to FIG. 4A, the control system 180 (FIG. 1) can (i) actuate the actuator 123 of the first aspiration syringe 112 to withdraw the plunger 120 in the direction W through the barrel 121 of the first aspiration syringe 112 to generate vacuum pressure (e.g., negative pressure) within the barrel 121 and (ii) actuate the actuator 123 of the second aspiration syringe 113 to withdraw the plunger 120 in the direction W through the barrel 121 of the second aspiration syringe 112 to generate vacuum pressure (e.g., negative pressure) within the barrel 121. Because the first aspiration syringe valve 116 and the second aspiration syringe valve 117 are closed, vacuum is charged within the barrels 121 of the first and second aspiration syringes 112, 113 (e.g., a negative pressure is maintained) before the first and second aspiration syringes 112, 113 are fluidly connected to the aspiration lumen 104 of the aspiration catheter 102 (FIG. 1).

Referring to FIG. 4B, the control system 180 (FIG. 1) can then open the first aspiration syringe valve 116, the second aspiration syringe valve 117, and the aspiration inlet valve 114 to fluidly connect the barrels 121 of the first and second aspiration syringes 112, 113 to the aspiration lumen 104 of the aspiration catheter 102 (FIG. 1) via the aspiration inlet tube 118 and the aspiration connection tubing 111. The first and second aspiration syringe valves 116, 117 can be opened simultaneously or substantially simultaneously at the same time as the aspiration inlet valve 114, before the aspiration inlet valve 114 is opened, or after the aspiration inlet valve 114 is opened. Accordingly, the vacuum stored in the barrels 121 of the first and second aspiration syringes 112, 113 is applied to the aspiration lumen 104 of the aspiration catheter 102 (FIG. 1) to aspirate clot material 490 and blood 492 through the aspiration lumen 104 of the aspiration catheter 102, through the aspiration inlet tube 118, through the aspiration inlet valve 114, through the aspiration connection tubing 111, through the first aspiration syringe valve 116 and into the barrels 121 of the first and second aspiration syringes 112, 113. In some embodiments, compared to the embodiment described in detail with reference to FIG. 3C, the combined vacuum (e.g., 120 cc) provided by both of the first and second aspiration syringes 112, 113—rather than just one—can provide a larger clot aspiration force.

Referring to FIG. 4C, the control system 180 (FIG. 1) can then (i) close the aspiration inlet valve 114 to fluidly disconnect the first and second aspiration syringes 112, 113 from the aspiration catheter 102 (FIG. 1), (ii) close the second aspiration syringe valve 117 to fluidly disconnect the barrel 121 of the second aspiration syringe 112 from the aspiration connection tubing 111, and (iii) open the aspiration outlet valve 115 to fluidly connect the first aspiration syringe 112 to the filter assembly 130 via the aspiration connection tubing 111 and the aspiration outlet tube 119. The control system 180 can further actuate the actuator 123 of the first aspiration syringe 112 to depress the plunger 120 through the barrel 121 of the first aspiration syringe 112 to drive the clot material 490 in the first aspiration syringe 112 and the blood 492 through the first aspiration syringe valve 116, through the aspiration connection tubing 111, through the aspiration outlet tube 119, and into the chamber 134 of the filter assembly 130. The clot material 490 and the blood 492 are received through the inlet 135 of the filter assembly 130, and gravity causes the clot material 390 and the blood 392 to move downward through the chamber 134 toward the filter tray 136, the collection component 137, and the filter 139, as described in detail above with reference to FIGS. 1 and 3E.

Referring to FIG. 4D, the control system 180 (FIG. 1) can then (i) close the first aspiration syringe valve 116 to fluidly disconnect the barrel 121 of the first aspiration syringe 112 from the aspiration connection tubing 111 and (ii) open the second aspiration syringe valve 117 to fluidly connect the second aspiration syringe 113 to the filter assembly 130 via the aspiration connection tubing 111 and the aspiration outlet tube 119. The control system 180 can further actuate the actuator 123 of the second aspiration syringe 113 to depress the plunger 120 through the barrel 121 of the second aspiration syringe 113 to drive the clot material 490 in the second aspiration syringe 113 and the blood 492 through the second aspiration syringe valve 117, through the aspiration connection tubing 111, through the aspiration outlet tube 119, and into the chamber 134 of the filter assembly 130.

Referring to FIG. 4E, the control system 180 (FIG. 1) can then (i) open the filter valve 141 of the filter assembly 130 to fluidly connect the filter conduit 140 of the filter assembly 130 to the reinfusion connection tubing 151 and (ii) open the reinfusion syringe valve 157 to fluidly connect the reinfusion syringe 112 to the filter assembly 130 via the reinfusion connection tubing 151. The control system 180 can also optionally close the first aspiration syringe valve 116 and the aspiration outlet valve 115 to fluidly disconnect the filter assembly 130 from the aspiration assembly 110. The control system 180 can further actuate the actuator 163 of the reinfusion syringe 153 to withdraw the plunger 160 through the barrel 161 of the reinfusion syringe 153 in the direction W to generate vacuum force to pull the blood 492 from the filter assembly 130 into the barrel 161 of the reinfusion syringe 153. As described in detail above with reference to FIGS. 1 and 3G, the filter 139 filters out smaller portions of the clot material 490 from the blood 492 such that the blood 492 is suitable for reinfusion into the patient. The filtered blood 492 can then be reinfused into a patient and the system 100 flushed as described in detail above with reference to FIGS. 3H-3N.

In the illustrated embodiment, the reinfusion syringe 153 is large enough to hold all of the blood 492 collected in the filter assembly 130 from the first and second aspiration syringes 112, 113. For example, the first and second aspiration syringes 112, 113 can be 60 cc syringes while the reinfusion syringe 153 can be a 120 cc syringe. In other embodiments, the steps of drawing the blood 492 into the reinfusion syringe 153 from the filter assembly 130 and subsequently reinfusing the blood 492 into the patient can be broken into multiple steps if the reinfusion syringe 153 is not large enough to hold all of the blood 492. For example, after receiving and reinfusing a portion of the blood 492, the control system 180 can (i) close the reinfusion outlet valve 155, open the filter valve 141, and actuate the actuator 163 of the reinfusion syringe 153 to draw a remaining portion of the blood 492 into the barrel 161 of the reinfusion syringe 153 and then (ii) open the reinfusion outlet valve 155, close the filter valve 141 of the filter assembly 130, and actuate the actuator 163 of the reinfusion syringe 153 to drive the remaining portion of the blood 492 into the reinfusion catheter 106 (FIG. 1). Flushing operations (FIGS. 3J-3N) can occur after all of the blood 492 is filtered through the filter assembly 130 and reinfused into the reinfusion catheter 106.

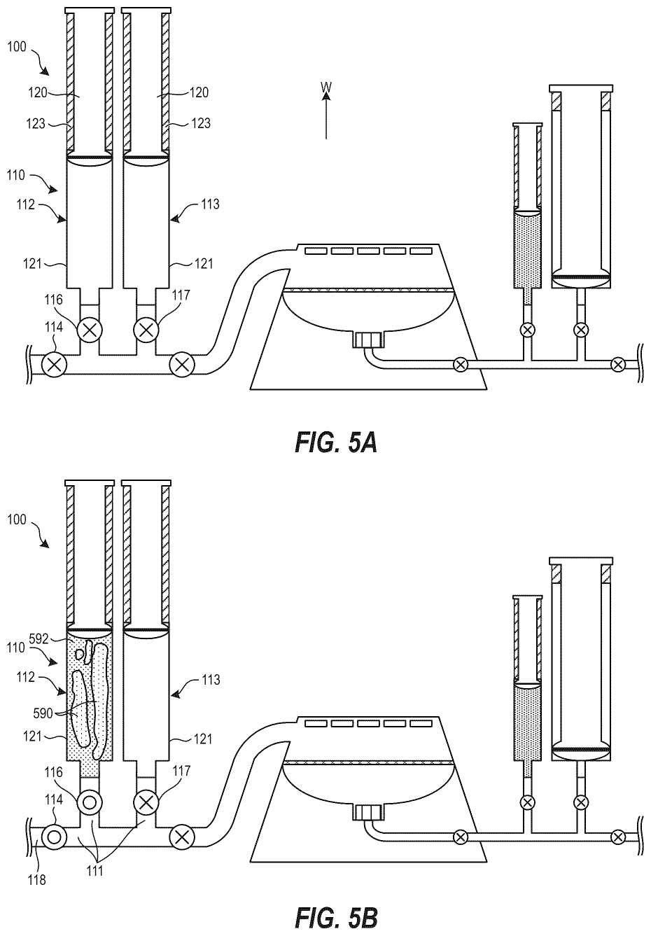

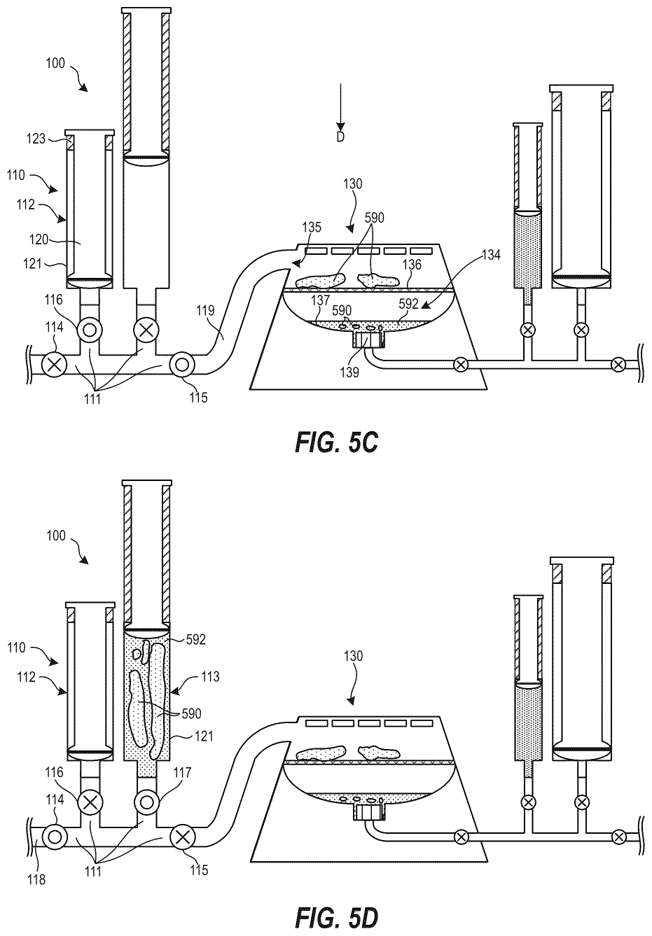

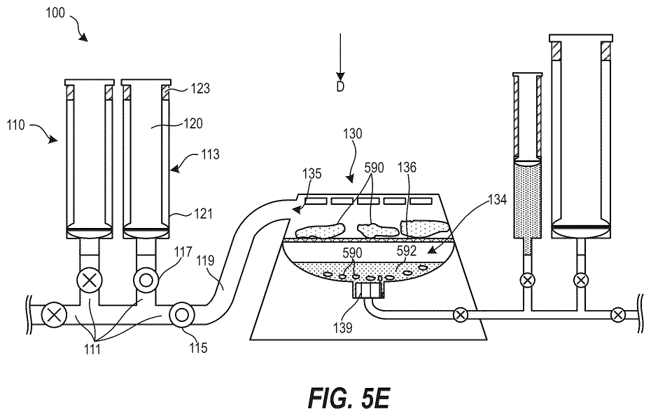

FIGS. 5A-5E are partially-schematic side cross-sectional views of the clot treatment system 100 of FIG. 1 illustrating a method or sequence performed by the system 100 of aspirating clot material and blood from a patient, filtering the blood from the clot material, reinfusing the filtered blood into the patient, flushing the filter assembly 130, and/or flushing the reinfusion catheter 106 in accordance with additional embodiments with the present technology. The system 100 can initially have the initial configuration shown in FIG. 3A. Then, referring to FIG. 5A, the control system 180 (FIG. 1) can (i) actuate the actuator 123 of the first aspiration syringe 112 to withdraw the plunger 120 in the direction W through the barrel 121 of the first aspiration syringe 112 to generate vacuum pressure (e.g., negative pressure) within the barrel 121 and (ii) actuate the actuator 123 of the second aspiration syringe 113 to withdraw the plunger 120 in the direction W through the barrel 121 of the second aspiration syringe 112 to generate vacuum pressure (e.g., negative pressure) within the barrel 121. Because the first aspiration syringe valve 116 and the second aspiration syringe valve 117 are closed, vacuum is charged within the barrels 121 of the first and second aspiration syringes 112, 113 (e.g., a negative pressure is maintained) before the first and second aspiration syringes 112, 113 are fluidly connected to the aspiration lumen 104 of the aspiration catheter 102 (FIG. 1).

Referring to FIG. 5B, the control system 180 (FIG. 1) can then open the first aspiration syringe valve 116 and the aspiration inlet valve 114 to fluidly connect the barrel 121 of the first aspiration syringe 112 to the aspiration lumen 104 of the aspiration catheter 102 (FIG. 1) via the aspiration inlet tube 118 and the aspiration connection tubing 111. The first aspiration syringe valve 116 can be opened at the same time as the aspiration inlet valve 114, before the aspiration inlet valve 114 is opened, or after the aspiration inlet valve 114 is opened. Accordingly, the vacuum stored in the barrel 121 of the first aspiration syringe 112 is applied to the aspiration lumen 104 of the aspiration catheter 102 (FIG. 1) to aspirate clot material 590 and blood 592 through the aspiration lumen 104 of the aspiration catheter 102, through the aspiration inlet tube 118, through the aspiration inlet valve 114, through the aspiration connection tubing 111, through the first aspiration syringe valve 116 and into the barrel 121 of the first aspiration syringe 112. The second aspiration syringe valve 117 can remain closed such that vacuum is maintained in the second aspiration syringe 113.

Referring to FIG. 5C, the control system 180 (FIG. 1) can then (i) close the aspiration inlet valve 114 to fluidly disconnect the first aspiration syringe 112 from the aspiration catheter 102 (FIG. 1), and (ii) open the aspiration outlet valve 115 to fluidly connect the first aspiration syringe 112 to the filter assembly 130 via the aspiration connection tubing 111 and the aspiration outlet tube 119. The control system 180 can further actuate the actuator 123 of the first aspiration syringe 112 to depress the plunger 120 through the barrel 121 of the first aspiration syringe 112 to drive the clot material 590 in the first aspiration syringe 112 and the blood 592 through the first aspiration syringe valve 116, through the aspiration connection tubing 111, through the aspiration outlet tube 119, and into the chamber 134 of the filter assembly 130. The clot material 590 and the blood 592 are received through the inlet 135 of the filter assembly 130, and gravity causes the clot material 590 and the blood 592 to move downward through the chamber 134 toward the filter tray 136, the collection component 137, and the filter 139, as described in detail above with reference to FIGS. 1 and 3E.

Referring to FIG. 5D, the control system 180 (FIG. 1) can then (i) close the first aspiration syringe valve 116 to fluidly disconnect the first aspiration syringe 112 from the aspiration connection tubing 111 and (ii) close the aspiration outlet valve 115 to fluidly disconnect the aspiration assembly 110 from the filter assembly 130. The control system 180 can then open the second aspiration syringe valve 117 and the aspiration inlet valve 114 to fluidly connect the barrel 121 of the second aspiration syringe 113 to the aspiration lumen 104 of the aspiration catheter 102 (FIG. 1) via the aspiration inlet tube 118 and the aspiration connection tubing 111. The second aspiration syringe valve 117 can be opened at the same time as the aspiration inlet valve 114, before the aspiration inlet valve 114 is opened, or after the aspiration inlet valve 114 is opened. Accordingly, the vacuum stored in the barrel 121 of the second aspiration syringe 113 is applied to the aspiration lumen 104 of the aspiration catheter 102 (FIG. 1) to aspirate additional clot material 590 and blood 592 through the aspiration lumen 104 of the aspiration catheter 102, through the aspiration inlet tube 118, through the aspiration inlet valve 114, through the aspiration connection tubing 111, through the second aspiration syringe valve 117 and into the barrel 121 of the second aspiration syringe 113.

Referring to FIG. 5E, the control system 180 (FIG. 1) can then (i) close the aspiration inlet valve 114 to fluidly disconnect the second aspiration syringe 113 from the aspiration catheter 102 (FIG. 1) and (ii) open the aspiration outlet valve 115 to fluidly connect the second aspiration syringe 113 to the filter assembly 130 via the aspiration connection tubing 111 and the aspiration outlet tube 119. The control system 180 can further actuate the actuator 123 of the second aspiration syringe 113 to depress the plunger 120 through the barrel 121 of the second aspiration syringe 113 to drive the additional clot material 590 in the second aspiration syringe 113 and the additional blood 592 through the first aspiration syringe valve 116, through the aspiration connection tubing 111, through the aspiration outlet tube 119, and into the chamber 134 of the filter assembly 130. The additional clot material 590 and the additional blood 592 are received through the inlet 135 of the filter assembly 130, and gravity causes the additional clot material 590 and the additional blood 592 to move downward through the chamber 134 toward the filter tray 136, the collection component 137, and the filter 139, as described in detail above with reference to FIGS. 1 and 3E.

At this point, the system 100 is configured as shown in FIG. 4D, and the system 100 can continue to filter the blood 592 collected in the filter assembly 130, reinfuse the blood 592 into the vasculature of the patient, and flush the system 100 as described in detail above with reference to FIGS. 3H-3N and 4E.

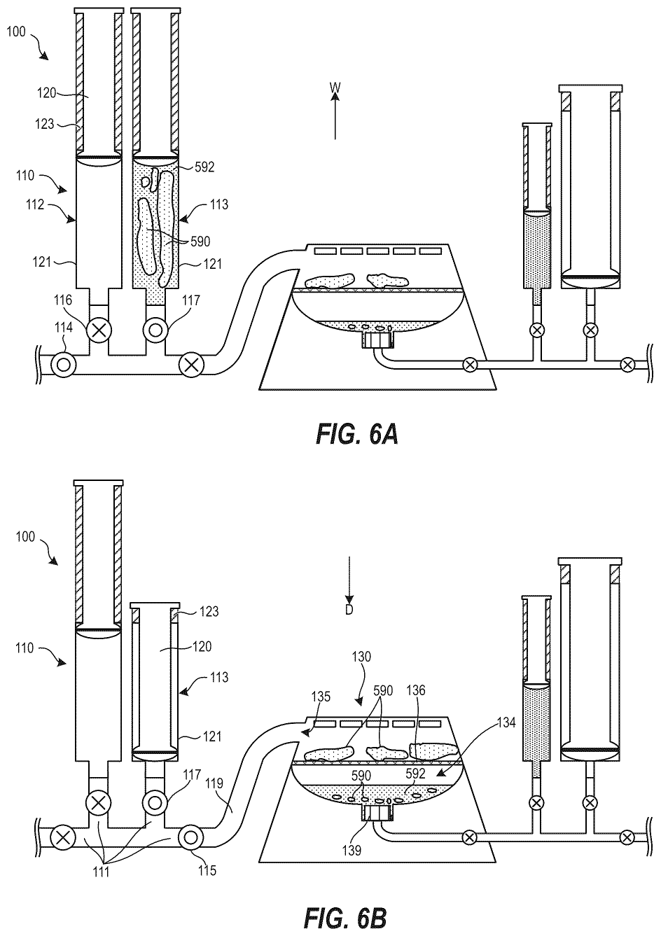

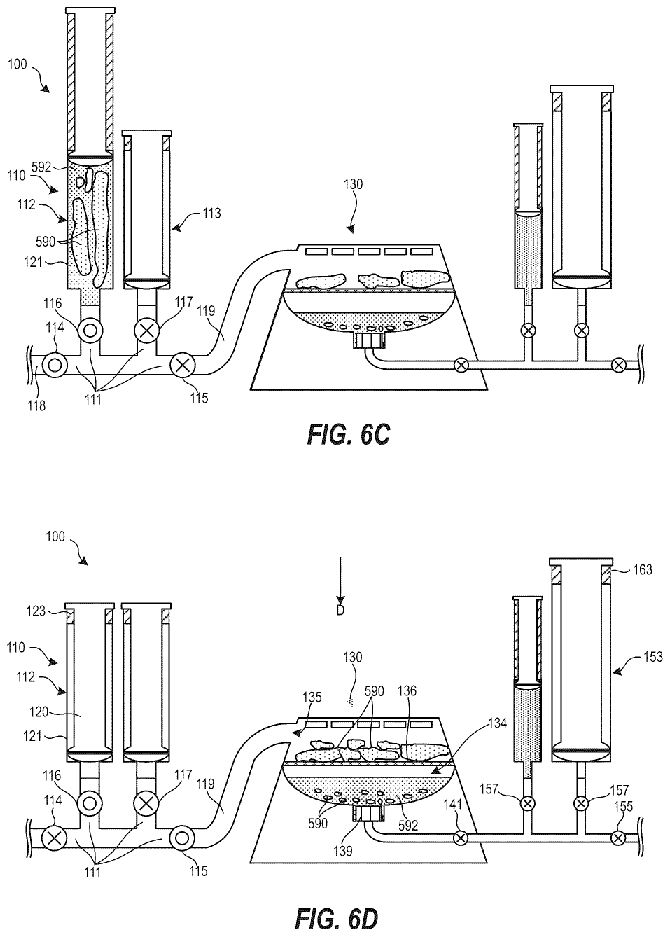

FIGS. 6A-6D are partially-schematic side cross-sectional views of the clot treatment system 100 of FIG. 1 illustrating a method or sequence performed by the system 100 of aspirating clot material and blood from a patient, filtering the blood from the clot material, reinfusing the filtered blood into the patient, flushing the filter assembly 130, and/or flushing the reinfusion catheter 106 in accordance with additional embodiments with the present technology. The method/sequence can proceed generally similarly or identically to the method/sequence described in detail above with reference to FIGS. 5A-5D. However, referring to FIG. 6A, when the second aspiration syringe valve 117 and the aspiration inlet valve 114 are opened to aspirate the additional clot material 590 and the additional blood 592 into the barrel 121 of the second aspiration syringe 113 as shown in FIG. 5D, the control system 180 can again actuate the actuator 123 of the first aspiration syringe 112 to withdraw the plunger 120 in the direction W through the barrel 121 of the first aspiration syringe 112 to generate vacuum pressure (e.g., negative pressure) within the barrel 121 with the first aspiration syringe valve 116 in a closed state. The actuator 123 can be actuated at the same time as the valves 114, 117 are opened, or before or after.

Referring to FIG. 6B, the control system 180 (FIG. 1) can then (i) close the aspiration inlet valve 114 to fluidly disconnect the second aspiration syringe 113 from the aspiration catheter 102 (FIG. 1) and (ii) open the aspiration outlet valve 115 to fluidly connect the second aspiration syringe 113 to the filter assembly 130 via the aspiration connection tubing 111 and the aspiration outlet tube 119. The control system 180 can further actuate the actuator 123 of the second aspiration syringe 113 to depress the plunger 120 through the barrel 121 of the second aspiration syringe 113 to drive the additional clot material 590 in the second aspiration syringe 113 and the additional blood 592 through the first aspiration syringe valve 116, through the aspiration connection tubing 111, through the aspiration outlet tube 119, and into the chamber 134 of the filter assembly 130. The additional clot material 590 and the additional blood 592 are received through the inlet 135 of the filter assembly 130, and gravity causes the additional clot material 590 and the additional blood 592 to move downward through the chamber 134 toward the filter tray 136, the collection component 137, and the filter 139, as described in detail above with reference to FIGS. 1 and 3E.

Referring to FIG. 6C, the control system 180 (FIG. 1) can then (i) close the second aspiration syringe valve 117 to fluidly disconnect the second aspiration syringe 113 from the aspiration connection tubing 111 and (ii) close the aspiration outlet valve 115 to fluidly disconnect the aspiration assembly 110 from the filter assembly 130. The control system 180 can then open the first aspiration syringe valve 116 and the aspiration inlet valve 114 to fluidly connect the barrel 121 of the first aspiration syringe 112 to the aspiration lumen 104 of the aspiration catheter 102 (FIG. 1) via the aspiration inlet tube 118 and the aspiration connection tubing 111. Accordingly, the vacuum stored in the barrel 121 of the first aspiration syringe 112 is applied to the aspiration lumen 104 of the aspiration catheter 102 (FIG. 1) to aspirate additional clot material 590 and blood 592 through the aspiration lumen 104 of the aspiration catheter 102, through the aspiration inlet tube 118, through the aspiration inlet valve 114, through the aspiration connection tubing 111, through the first aspiration syringe valve 116, and into the barrel 121 of the first aspiration syringe 112.

Referring to FIG. 6D, the control system 180 (FIG. 1) can then (i) close the aspiration inlet valve 114 to fluidly disconnect the first aspiration syringe 112 from the aspiration catheter 102 (FIG. 1), and (ii) open the aspiration outlet valve 115 to fluidly connect the first aspiration syringe 112 to the filter assembly 130 via the aspiration connection tubing 111 and the aspiration outlet tube 119. The control system 180 can further actuate the actuator 123 of the first aspiration syringe 112 to depress the plunger 120 through the barrel 121 of the first aspiration syringe 112 to drive the additional clot material 590 in the first aspiration syringe 112 and the additional blood 592 through the first aspiration syringe valve 116, through the aspiration connection tubing 111, through the aspiration outlet tube 119, and into the chamber 134 of the filter assembly 130. The clot material 590 and the blood 592 are received through the inlet 135 of the filter assembly 130, and gravity causes the clot material 590 and the blood 592 to move downward through the chamber 134 toward the filter tray 136, the collection component 137, and the filter 139, as described in detail above with reference to FIGS. 1 and 3E.

At this point, the system 100 is generally configured as shown in FIGS. 4D and 5E (albeit with more of the clot material 590 and the blood 592 in the filter assembly 130), and the system 100 can continue to filter the blood 592 collected in the filter assembly 130, reinfuse the blood into the vasculature of the patient, and flush the system 100 as described in detail above with reference to FIGS. 3H-3N and 4E. In particular, based on the size of the reinfusion syringe 153, the system can control the valves 141 and 155-157 and the actuator 163 of the reinfusion syringe 153 to provide one or multiple filtering and reinfusion cycles.

While FIGS. 3A-6D provide several representative methods/sequences, the system 100 can repeat such sequences in any order, and/or the system 100 can be programmed to carry out other sequences. For example, the system 100 can be configured to do 4, 5, or greater aspirations and filtrations in any combination of amounts or volumes.

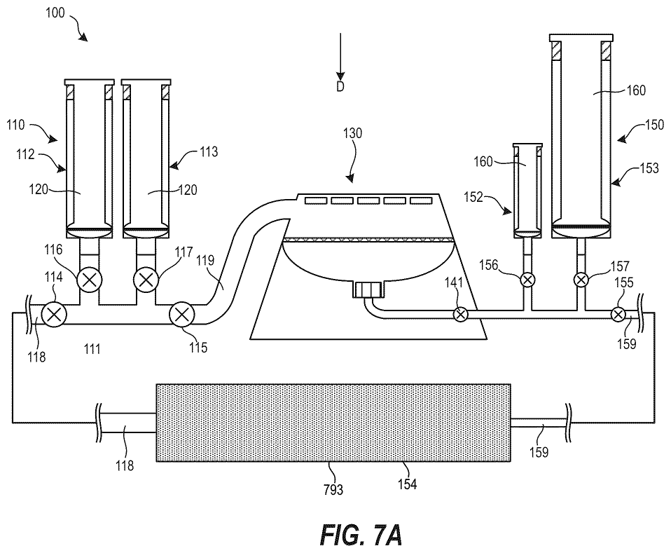

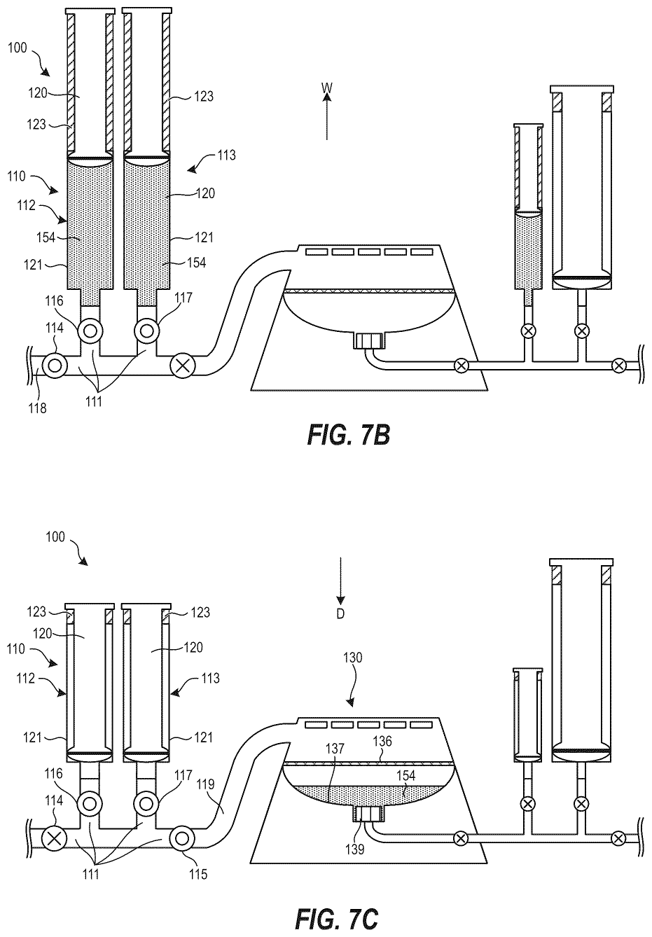

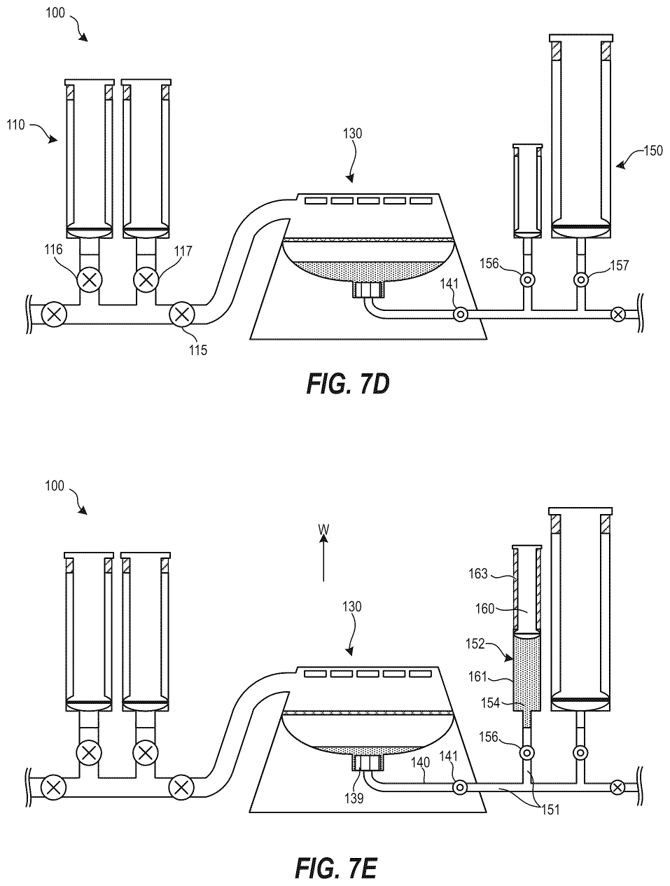

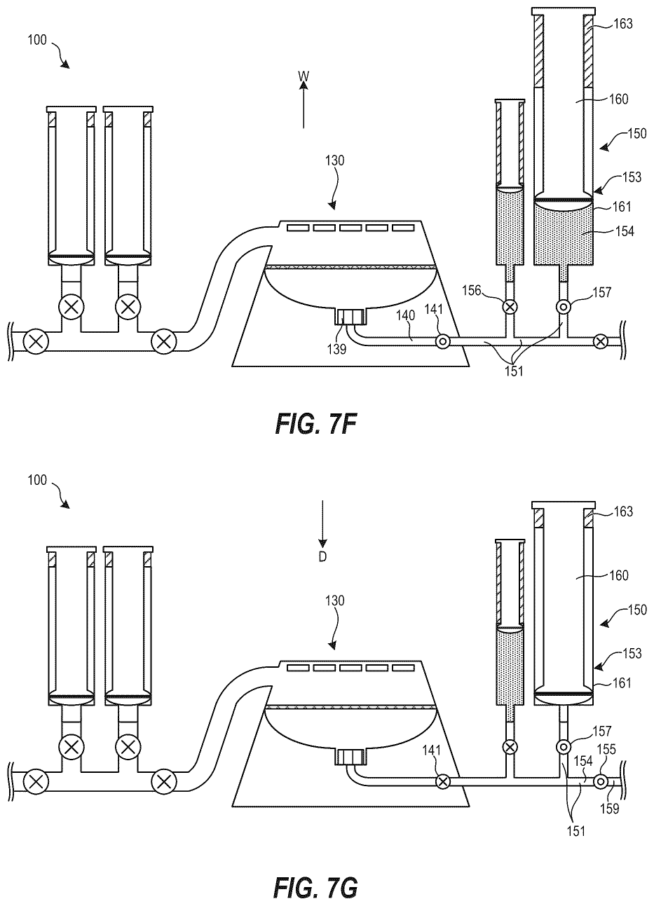

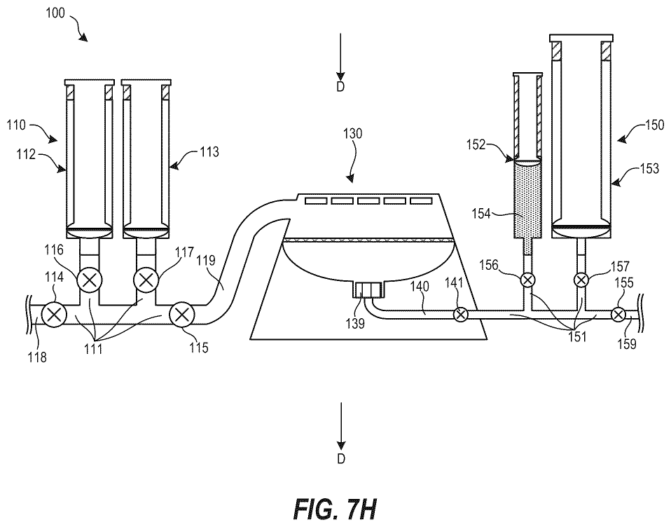

FIGS. 7A-7H are partially-schematic side cross-sectional views of the clot treatment system 100 of FIG. 1 illustrating a method or sequence performed by the system 100 for priming the system to have the initial configuration shown in FIG. 3A in accordance with embodiments of the present technology. Referring to FIG. 7A, initially the valves 114-117, 141, 155-167 can each be closed, the first and second aspiration syringes 112, 113 can be in a depressed state with the plungers 120 thereof depressed in the direction D, and the flushing and reinfusion syringes 152, 153 can be in a depressed state with the plungers 160 thereof depressed in the direction D. In the illustrated embodiment, the aspiration inlet tube 118 and the reinfusion outlet tube 159 are each placed in a reservoir 793 (e.g., bowl) containing the flushing fluid 154.

Referring to FIG. 7B, the control system 180 (FIG. 1) can (i) open the aspiration inlet valve 114, the first aspiration syringe valve 116, and the second aspiration syringe valve 117 and then (ii) actuate the actuators 123 of the first and second aspiration syringes 112, 112 to withdraw the plungers 120 in the direction W to generate vacuum pressure to draw a portion of the flushing fluid 154 from the reservoir 793 (FIG. 7A) through the aspiration inlet tube 118, through the aspiration connection tubing 111, and through the first and second aspiration syringe valves 116, 117 into the barrels 121 of the first and second aspiration syringes 112, 113.

Referring to FIG. 7C, the control system 180 (FIG. 1) can then (i) close the aspiration inlet valve 114 and open the aspiration outlet valve 115 and then (ii) actuate the actuators 123 of the first and second aspiration syringes 112, 112 to depress the plungers 120 in the direction D to drive the flushing fluid 154 from the barrels 121 of the first and second aspiration syringes 112, 113, through the first and second aspiration syringe valves 116, 117, through the aspiration connection tubing 111, through the aspiration outlet valve 115, through the aspiration outlet tube 119 and into the chamber 134 of the filter assembly 130. The flushing fluid 154 can pass through the filter tray and collect along the collection component 137 and surround the filter 139.

Referring to FIG. 7D, the control system 180 (FIG. 1) can then (i) close the first and second aspiration syringe valves 116, 117 and the aspiration outlet valve 115, (ii) open the filter valve 141 of the filter assembly 130, and (iii) open the flushing syringe valve 156 and the reinfusion syringe valve 157.

Referring to FIG. 7E, the control system 180 can then actuate the actuator 163 of the flushing syringe 152 to withdraw the plunger 160 of the flushing syringe 152 in the direction W to generate vacuum pressure to draw a portion of the flushing fluid 154 from the filter assembly 130, through the filter 139, through the filter conduit 140, through the filter valve 141, through the reinfusion connection tubing 151, through the flushing syringe valve 156, and into the barrel 161 of the flushing syringe 152.

Referring to FIG. 7F, the control system 180 (FIG. 1) can then (i) close the flushing syringe valve 156 and (ii) actuate the actuator 163 of the reinfusion syringe 153 to withdraw the plunger 160 through the barrel 161 of the reinfusion syringe 153 in the direction W to generate vacuum force to pull the remainder of the flushing fluid 154 from the filter assembly 130, through the filter 139, through the filter conduit 140, through the filter valve 141, through the reinfusion connection tubing 151, through the reinfusion syringe valve 157, and into the barrel 161 of the reinfusion syringe 153.

Referring to FIG. 7G, the control system 180 (FIG. 1) can then (i) close the filter valve 141, (ii) open the reinfusion outlet valve 155, and (iii) actuate the actuator 163 of the reinfusion syringe 153 to depress the plunger 160 through the barrel 161 of the reinfusion syringe 153 to drive the flushing fluid 154 therein through the reinfusion syringe valve 157, through the reinfusion connection tubing 151, through the reinfusion outlet valve 155, through the reinfusion outlet tube 159, and into the reservoir 793 (FIG. 7A).

Finally, referring to FIG. 7H, the control system 180 (FIG. 1) can close the reinfusion syringe valve 157 and the reinfusion outlet valve 155 of the reinfusion and flushing assembly 150. At this point, the system 100 is in the initial configuration illustrated in FIG. 3A and ready to perform any of the various operations, sequences, and/or methods described herein (e.g., with reference to FIGS. 3A-6D). In some aspects of the present technology, cycling the flushing fluid 154 through the various tubing components 118, 111, 119, 140, 151, and 159 of the aspiration, filter, and reinfusion and flushing assemblies 110, 130, 150, each of the valves 114-117, 141, 155-167, each of the syringes 112, 113, 152, 153, and the filter assembly 130 can remove any contaminants and/or air from these components-priming the system for subsequent clot removal and blood reinfusion operations.

### III. SELECTED EMBODIMENTS OF SYSTEM PROGRAMMING SEQUENCES

Referring to FIG. 1, in some embodiments the computer-readable medium 182 of the control system 180 can store a programming sequence/algorithm that, when executed by the processor 184 of the control system 180, can cause the processor 184 to control the system 100 to execute a sequence/algorithm of mechanical operations to perform a method, such any of the methods described in detail above with reference to FIGS. 3A-7H.

For example, Table 1 below provides an overview of a programming sequence of mechanical steps/actions to accomplish the same or a substantially similar method to that illustrated in and described in detail with reference to FIGS. 3A-3N—comprising, for example, a single syringe aspiration (e.g., 60 cc) followed by a blood reinfusion (e.g., 60 cc blood reinfusion). Table 1 also describes any resultant fluid flow through the system 100 caused by the mechanical step/action. The steps/actions of the programming sequence do not necessarily need to be performed in the listed sequence. Likewise, some of the steps (e.g., opening/closing of valves, retracting/advancing syringe plungers) can occur at least partially simultaneously to, for example, improve the speed of the sequence. The listed sequence can be performed after the system 100 is in the initial configuration described in detail with reference to FIGS. 3A and 7A-7G.

TABLE 1

| Step/Action of the System 100 | Fluid Flow Through the System 100 |
|---|---|
| Open the aspiration inlet valve 114. | |
| Withdraw the plunger 120 of the first aspiration | Vacuum generation in the barrel 121 of the first |
| syringe 112. | aspiration syringe 112. |
| Open the first aspiration syringe valve 116. | Clot material and blood flow into the barrel 121 |
| | of the first aspiration syringe 112 from the |
| | aspiration catheter 102 from the vasculature of |
| | the patient. |
| Close the aspiration inlet valve 114. | |
| Open the aspiration outlet valve 115. | |
| Depress the plunger 120 of the of the first | The clot material and the blood in the first |
| aspiration syringe 112. | aspiration syringe 112 flow into the filter |
| | assembly 130 from the aspiration assembly 110. |
| Close the aspiration outlet valve 115. | |
| Open the filter valve 141. | |
| Open the reinfusion syringe valve 157. | |
| Withdraw the plunger 160 of the reinfusion | Filtered blood flows into the barrel 161 of the |
| syringe 153. | reinfusion syringe 153 from the filter assembly |
| | 130. |
| Close the filter valve 141. | |
| Open the reinfusion outlet valve 155. | |
| Depress the plunger 160 of the reinfusion | The filtered blood flows into the reinfusion |
| syringe 153. | catheter 106 from the barrel 161 of the |
| | reinfusion syringe 153 for reinfusion into the |
| | vasculature of the patient. |
| Close the reinfusion outlet valve 155. | |
| Close the reinfusion syringe valve 157. | |
| Open the filter valve 141. | |
| Open the flushing syringe valve 156. | |
| Partially depress the plunger 160 of the | A portion of the flushing fluid 154 flows into the |
| reinfusion syringe 153. | filter assembly 130 from the barrel 161 of the |
| | flushing syringe 152 to flush the filter 139. |
| Close the filter valve 141. | |
| Open the reinfusion outlet valve 155. | |
| Further depress the plunger 160 of the reinfusion | A portion of the flushing fluid 154 flows into the |
| syringe 153. | reinfusion catheter 106 from the barrel 161 of |
| | the flushing syringe 152 to push any remaining |
| | filtered blood out of the reinfusion catheter 106. |
| Close all valves. | |

For example, Table 2 below provides an overview of a programming sequence of mechanical steps/actions to accomplish the same or a substantially similar method to that illustrated in and described in detail with reference to FIGS. 4A-4E—comprising, for example, a simultaneous two-syringe aspiration (e.g., 120 cc aspiration) followed by a blood reinfusion (e.g., 120 cc blood reinfusion). The listed programming sequence assumes that the first and second aspiration syringes 112, 113 and the reinfusion syringe 153 have the same or substantially similar volume (e.g., 60 cc). Table 2 also describes any resultant fluid flow through the system 100 caused by the mechanical step/action. The steps/actions of the programming sequence do not necessarily need to be performed in the listed sequence. Likewise, some of the steps (e.g., opening/closing of valves, retracting/advancing syringe plungers) can occur at least partially simultaneously to, for example, improve the speed of the sequence. The listed sequence can be performed after the system 100 is in the initial configuration described in detail with reference to FIGS. 3A and 7A-7G.

TABLE 2

| Step/Action of the System 100 | Fluid Flow Through the System 100 |
|---|---|
| Open the aspiration inlet valve 114. | |
| Withdraw the plunger 120 of the first aspiration | Vacuum generation in the barrel 121 of the first |
| syringe 112. | aspiration syringe 112. |
| Withdraw the plunger 120 of the second | Vacuum generation in the barrel 121 of the |
| aspiration syringe 113. | second aspiration syringe 113. |
| Open the first aspiration syringe valve 116 and | Clot material and blood flow into the barrels 121 |
| the second aspiration syringe valve 117 | of the first and second aspiration syringes 112, |
| simultaneously or substantially simultaneously. | 113 from the aspiration catheter 102 from the |
| | vasculature of the patient. |
| Close the aspiration inlet valve 114. | |
| Close the second aspiration syringe valve 117. | |
| Open the aspiration outlet valve 115. | |
| Depress the plunger 120 of the of the first | The clot material and the blood in the first |
| aspiration syringe 112. | aspiration syringe 112 flow into the filter |
| | assembly 130 from the aspiration assembly 110. |
| Close the first aspiration syringe valve 116. | |
| Open the second aspiration syringe valve 117. | |
| Depress the plunger 120 of the of the second | The clot material and the blood in the second |
| aspiration syringe 113. | aspiration syringe 113flow into the filter |
| | assembly 130 from the aspiration assembly 110. |
| Close the second aspiration syringe valve 117. | |
| Close the aspiration outlet valve 115. | |
| Open the filter valve 141. | |
| Open the reinfusion syringe valve 157. | |
| Withdraw the plunger 160 of the reinfusion | Filtered blood flows into the barrel 161 of the |
| syringe 153. | reinfusion syringe 153 from the filter assembly |
| | 130. |
| Close the filter valve 141. | |
| Open the reinfusion outlet valve 155. | |
| Depress the plunger 160 of the reinfusion | The filtered blood flows into the reinfusion |
| syringe 153. | catheter 106 from the barrel 161 of the |
| | reinfusion syringe 153 for reinfusion into the |
| | vasculature of the patient. |
| Open the filter valve 141. | |
| Close the reinfusion outlet valve 155. | |
| Withdraw the plunger 160 of the reinfusion | Additional filtered blood flows into the barrel |
| syringe 153. | 161 of the reinfusion syringe 153 from the filter |
| | assembly 130. |
| Close the filter valve 141. | |
| Open the reinfusion outlet valve 155. | |
| Depress the plunger 160 of the reinfusion | The additional filtered blood flows into the |
| syringe 153. | reinfusion catheter 106 from the barrel 161 of |
| | the reinfusion syringe 153 for reinfusion into the |
| | vasculature of the patient. |
| Open the flushing syringe valve 156. | |
| Close the reinfusion syringe valve 157. | |
| Partially depress the plunger 160 of the | A portion of the flushing fluid 154 flows into the |
| reinfusion syringe 153. | reinfusion catheter 106 from the barrel 161 of |
| | the flushing syringe 152 to push any remaining |
| | filtered blood out of the reinfusion catheter 106. |
| Close the reinfusion outlet valve 155. | |
| Open the filter valve 141. | |
| Further depress the plunger 160 of the reinfusion | Another portion of the flushing fluid 154 flows |
| syringe 153. | into the filter assembly 130 from the barrel 161 |
| | of the flushing syringe 152 to flush the filter 139. |
| Close all valves. | |

For example, Table 3 below provides an overview of a programming sequence of mechanical steps/actions to accomplish the same or a substantially similar method to that illustrated in and described in detail with reference to FIGS. 5A-5E—comprising, for example, back-to-back single syringe aspirations (e.g., back-to-back 60 cc aspirations) followed by a blood reinfusion (e.g., a 60 cc blood reinfusion). The listed programming sequence assumes that the first and second aspiration syringes 112, 113 and the reinfusion syringe 153 have the same or substantially similar volume (e.g., 60 cc). Table 3 also describes any resultant fluid flow through the system 100 caused by the mechanical step/action. The steps/actions of the programming sequence do not necessarily need to be performed in the listed sequence. Likewise, some of the steps (e.g., opening/closing of valves, retracting/advancing syringe plungers) can occur at least partially simultaneously to, for example, improve the speed of the sequence. The listed sequence can be performed after the system 100 is in the initial configuration described in detail with reference to FIGS. 3A and 7A-7G.

TABLE 3

| Step/Action of the System 100 | Fluid Flow Through the System 100 |
|---|---|
| Open the aspiration inlet valve 114. | |
| Withdraw the plunger 120 of the first aspiration | Vacuum generation in the barrel 121 of the first |
| syringe 112. | aspiration syringe 112. |
| Withdraw the plunger 120 of the second | Vacuum generation in the barrel 121 of the |
| aspiration syringe 113. | second aspiration syringe 113. |
| Open the first aspiration syringe valve 116. | Clot material and blood flow into the barrel 121 |
| | of the first aspiration syringe 112 from the |
| | aspiration catheter 102 from the vasculature of |
| | the patient. |
| Close the aspiration inlet valve 114. | |
| Open the aspiration outlet valve 115. | |
| Depress the plunger 120 of the of the first | The clot material and the blood in the first |
| aspiration syringe 112. | aspiration syringe 112 flow into the filter |
| | assembly 130 from the aspiration assembly 110. |
| Open the aspiration inlet valve 114. | |
| Close the aspiration outlet valve 115. | |
| Close the first aspiration syringe valve 116. | |
| Open the second aspiration syringe valve 117. | Clot material and blood flow into the barrel 121 |
| | of the second aspiration syringe 113 from the |
| | aspiration catheter 102 from the vasculature of |
| | the patient. |
| Close the aspiration inlet valve 114. | |
| Open the aspiration outlet valve 115. | |
| Depress the plunger 120 of the of the second | The clot material and the blood in the second |
| aspiration syringe 113. | aspiration syringe 113 flow into the filter |
| | assembly 130 from the aspiration assembly 110. |
| Close the second aspiration syringe valve 117. | |
| Close the aspiration outlet valve 115. | |
| Open the filter valve 141. | |
| Open the reinfusion syringe valve 157. | |
| Withdraw the plunger 160 of the reinfusion | Filtered blood flows into the barrel 161 of the |
| syringe 153. | reinfusion syringe 153 from the filter assembly |
| | 130. |
| Close the filter valve 141. | |
| Open the reinfusion outlet valve 155. | |
| Depress the plunger 160 of the reinfusion | The filtered blood flows into the reinfusion |
| syringe 153. | catheter 106 from the barrel 161 of the |
| | reinfusion syringe 153 for reinfusion into the |
| | vasculature of the patient. |
| Open the filter valve 141. | |
| Close the reinfusion outlet valve 155. | |
| Withdraw the plunger 160 of the reinfusion | Additional filtered blood flows into the barrel |
| syringe 153. | 161 of the reinfusion syringe 153 from the filter |
| | assembly 130. |
| Close the filter valve 141. | |
| Open the reinfusion outlet valve 155. | |
| Depress the plunger 160 of the reinfusion | The additional filtered blood flows into the |
| syringe 153. | reinfusion catheter 106 from the barrel 161 of |
| | the reinfusion syringe 153 for reinfusion into the |
| | vasculature of the patient. |
| Open the flushing syringe valve 156. | |
| Close the reinfusion syringe valve 157. | |
| Partially depress the plunger 160 of the | A portion of the flushing fluid 154 flows into the |
| reinfusion syringe 153. | reinfusion catheter 106 from the barrel 161 of |
| | the flushing syringe 152 to push any remaining |
| | filtered blood out of the reinfusion catheter 106. |
| Close the reinfusion outlet valve 155. | |
| Open the filter valve 141. | |
| Further depress the plunger 160 of the reinfusion | Another portion of the flushing fluid 154 flows |
| syringe 153. | into the filter assembly 130 from the barrel 161 |
| | of the flushing syringe 152 to flush the filter 139. |
| Close all valves. | |

For example, Table 4 below provides an overview of a programming sequence of mechanical steps/actions to accomplish the same or a substantially similar method to that illustrated in and described in detail with reference to FIGS. 6A-6D—comprising, for example, back-to-back-back single syringe aspirations (e.g., back-to-back-to-back 60 cc aspirations) followed by a blood reinfusion (e.g., a 180 cc blood reinfusion). The listed programming sequence assumes that the first and second aspiration syringes 112, 113 and the reinfusion syringe 153 have the same or substantially similar volume (e.g., 60 cc). Table 4 also describes any resultant fluid flow through the system 100 caused by the mechanical step/action. The steps/actions of the programming sequence do not necessarily need to be performed in the listed sequence. Likewise, some of the steps (e.g., opening/closing of valves, retracting/advancing syringe plungers) can occur at least partially simultaneously to, for example, improve the speed of the sequence. The listed sequence can be performed after the system 100 is in the initial configuration described in detail with reference to FIGS. 3A and 7A-7G.

TABLE 4

| Step/Action of the System 100 | Fluid Flow Through the System 100 |
|---|---|
| Open the aspiration inlet valve 114. | |
| Withdraw the plunger 120 of the first aspiration | Vacuum generation in the barrel 121 of the first |
| syringe 112. | aspiration syringe 112. |
| Withdraw the plunger 120 of the second | Vacuum generation in the barrel 121 of the |
| aspiration syringe 113. | second aspiration syringe 113. |
| Open the first aspiration syringe valve 116. | Clot material and blood flow into the barrel 121 |
| | of the first aspiration syringe 112 from the |
| | aspiration catheter 102 from the vasculature of |
| | the patient. |
| Close the aspiration inlet valve 114. | |
| Open the aspiration outlet valve 115. | |
| Depress the plunger 120 of the of the first | The clot material and the blood in the first |
| aspiration syringe 112. | aspiration syringe 112 flow into the filter |
| | assembly 130 from the aspiration assembly 110. |
| Open the aspiration inlet valve 114. | |
| Close the aspiration outlet valve 115. | |
| Close the first aspiration syringe valve 116. | |
| Open the second aspiration syringe valve 117. | Clot material and blood flow into the barrel 121 |
| | of the second aspiration syringe 113 from the |
| | aspiration catheter 102 from the vasculature of |
| | the patient. |
| Withdraw the plunger 120 of the first aspiration | Vacuum generation in the barrel 121 of the first |
| syringe 112. | aspiration syringe 112. |
| Close the aspiration inlet valve 114. | |
| Open the aspiration outlet valve 115. | |
| Depress the plunger 120 of the of the second | The clot material and the blood in the second |
| aspiration syringe 113. | aspiration syringe 113 flow into the filter |
| | assembly 130 from the aspiration assembly 110. |
| Open the aspiration inlet valve 114. | |
| Close the aspiration outlet valve 115. | |
| Close the second aspiration syringe valve 117. | |
| Open the first aspiration syringe valve 116. | Clot material and blood flow into the barrel 121 |
| | of the first aspiration syringe 112 from the |
| | aspiration catheter 102 from the vasculature of |
| | the patient. |
| Close the aspiration inlet valve 114. | |
| Open the aspiration outlet valve 115. | |
| Depress the plunger 120 of the of the first | The clot material and the blood in the first |
| aspiration syringe 112. | aspiration syringe 112 flow into the filter |
| | assembly 130 from the aspiration assembly 110. |
| Close the first aspiration syringe valve 116. | |
| Close the aspiration outlet valve 115. | |
| Open the filter valve 141. | |
| Open the reinfusion syringe valve 157. | |
| Withdraw the plunger 160 of the reinfusion | A first portion of filtered blood flows into the |
| syringe 153. | barrel 161 of the reinfusion syringe 153 from the |
| | filter assembly 130. |
| Close the filter valve 141. | |
| Open the reinfusion outlet valve 155. | |
| Depress the plunger 160 of the reinfusion | The first portion of filtered blood flows into the |
| syringe 153. | reinfusion catheter 106 from the barrel 161 of |
| | the reinfusion syringe 153 for reinfusion into the |
| | vasculature of the patient. |
| Open the filter valve 141. | |
| Close the reinfusion outlet valve 155. | |
| Withdraw the plunger 160 of the reinfusion | A second portion of filtered blood flows into the |
| syringe 153. | barrel 161 of the reinfusion syringe 153 from the |
| | filter assembly 130. |
| Close the filter valve 141. | |
| Open the reinfusion outlet valve 155. | |
| Depress the plunger 160 of the reinfusion | The second portion of filtered blood flows into |
| syringe 153. | the reinfusion catheter 106 from the barrel 161 |
| | of the reinfusion syringe 153 for reinfusion into |
| | the vasculature of the patient. |
| Open the filter valve 141. | |
| Close the reinfusion outlet valve 155. | |
| Withdraw the plunger 160 of the reinfusion | A third portion of filtered blood flows into the |
| syringe 153. | barrel 161 of the reinfusion syringe 153 from the |
| | filter assembly 130. |
| Close the filter valve 141. | |
| Open the reinfusion outlet valve 155. | |
| Depress the plunger 160 of the reinfusion | The third portion of filtered blood flows into the |
| syringe 153. | reinfusion catheter 106 from the barrel 161 of |
| | the reinfusion syringe 153 for reinfusion into the |
| | vasculature of the patient. |
| Open the flushing syringe valve 156. | |
| Close the reinfusion syringe valve 157. | |
| Partially depress the plunger 160 of the | A portion of the flushing fluid 154 flows into the |
| reinfusion syringe 153. | reinfusion catheter 106 from the barrel 161 of |
| | the flushing syringe 152 to push any remaining |
| | filtered blood out of the reinfusion catheter 106. |
| Close the reinfusion outlet valve 155. | |
| Open the filter valve 141. | |
| Further depress the plunger 160 of the reinfusion | Another portion of the flushing fluid 154 flows |
| syringe 153. | into the filter assembly 130 from the barrel 161 |
| | of the flushing syringe 152 to flush the filter 139. |
| Close all valves. | |

For example, Table 5 below provides an overview of a programming sequence of mechanical steps/actions to accomplish the same or a substantially similar method to that illustrated in and described in detail with reference to FIGS. 7A-7H—comprising, for example, a system priming method. Table 5 also describes any resultant fluid flow through the system 100 caused by the mechanical step/action. The steps/actions of the programming sequence do not necessarily need to be performed in the listed sequence. Likewise, some of the steps (e.g., opening/closing of valves, retracting/advancing syringe plungers) can occur at least partially simultaneously to, for example, improve the speed of the sequence. The listed sequence can be performed after the system 100 is in the initial configuration in which all the valves are closed, all the syringes are depressed, and the aspiration inlet tube 118 and the reinfusion outlet tube 159 are placed in the reservoir 793 of the flushing fluid 154 as shown in, for example, FIG. 7A.

TABLE 5

| Step/Action of the System 100 | Fluid Flow Through the System 100 |
|---|---|
| Open the aspiration inlet valve 114. | |
| Open the first aspiration syringe valve 116. | |
| Open the second aspiration syringe valve 117. | |
| Withdraw the plunger 120 of the first aspiration | The flushing fluid 154 flows into the barrel 121 |
| syringe 112. | of the first aspiration syringe 112. |
| Withdraw the plunger 120 of the second | The flushing fluid 154 flows into the barrel 121 |
| aspiration syringe 113. | of the second aspiration syringe 113. |
| Close the aspiration inlet valve 114. | |
| Open the aspiration outlet valve 115. | |
| Depress the plunger 120 of the of the first | The flushing fluid 154 in the first aspiration |
| aspiration syringe 112. | syringe 112 flows into the filter assembly 130 |
| | from the aspiration assembly 110. |
| Depress the plunger 120 of the of the second | The flushing fluid 154 in the second aspiration |
| aspiration syringe 113. | syringe 113 flows into the filter assembly 130 |
| | from the aspiration assembly 110. |
| Close the aspiration outlet valve 115. | |
| Close the first aspiration syringe valve 116. | |
| Close the second aspiration syringe valve 117. | |
| Open the filter valve 141. | |
| Open the flushing syringe valve 156. | |
| Open the reinfusion syringe valve 157. | |
| Withdraw the plunger 160 of the flushing | A portion of the flushing fluid 154 flows into the |
| syringe 152. | barrel 161 of the flushing syringe 152 from the |
| | filter assembly 130. |
| Close the flushing syringe valve 156. | |
| Withdraw the plunger 160 of the reinfusion | Another portion of the flushing fluid 154 flows |
| syringe 153. | into the barrel 161 of the reinfusion syringe 153 |
| | from the filter assembly 130. |
| Close the filter valve 141. | |
| Open the reinfusion outlet valve 155. | |
| Depress the plunger 160 of the reinfusion | The other portion of the flushing fluid 154 flows |
| syringe 153. | into the reservoir 739 from the barrel 161 of the |
| | flushing syringe 152. |
| Close all valves | |

### IV. SELECTED EMBODIMENTS OF ADDITIONAL CLOT TREATMENT SYSTEMS AND DEVICES

Referring to FIG. 1, in some embodiments one or more components of the system 100 need to be coupled to the control system 180 and, instead, can be operated manually by an operator (e.g., a surgeon, a surgical team member). For example, the reinfusion syringe 153 can be operated manually by an operator to ensure that the no air is received in the barrel 161 and subsequently reintroduced to the patient. Likewise, the entire aspiration assembly 110 may be operated manually while the filter assembly 130 and the reinfusion and flushing assembly 150 are controlled automatically by the control system 180, the entire reinfusion and flushing assembly 150 may be operated manually while the filter assembly 130 and the aspiration assembly 110 are controlled automatically by the control system 180, and so on.

Additionally, the system 100 could contain only one, 3, 4, or more aspiration syringes coupled to the aspiration connection tubing 111 of the aspiration assembly 110 via corresponding aspiration syringe valves. Each aspiration syringe and corresponding valve can be operated similarly to the first and second aspiration syringes 112, 113 described in detail herein to, for example, provide for larger combined aspiration forces, reduced aspiration cycle times, improved efficiency, etc. Similarly, the system 100 can contain 2, 3, 4, or more reinfusions syringes coupled to the reinfusion connection tubing 151 of the reinfusion and flushing assembly 150 via corresponding reinfusion syringe valves. Each reinfusion syringe and corresponding valve can be operated similarly to the reinfusion syringe 153 described in detail herein to, for example, provide for quicker filtration cycles, reduced filtration cycle times, improved efficiency, etc. Likewise, the system 100 can contain 2, 3, 4, or more flushing syringes coupled to the reinfusion connection tubing 151 of the reinfusion and flushing assembly 150 via corresponding flushing syringe valves. Each flushing syringe and corresponding valve can be operated similarly to the flushing syringe 152 described in detail herein to, for example, provide for quicker flushing cycles, reduced flushing cycle times, improved efficiency, etc.

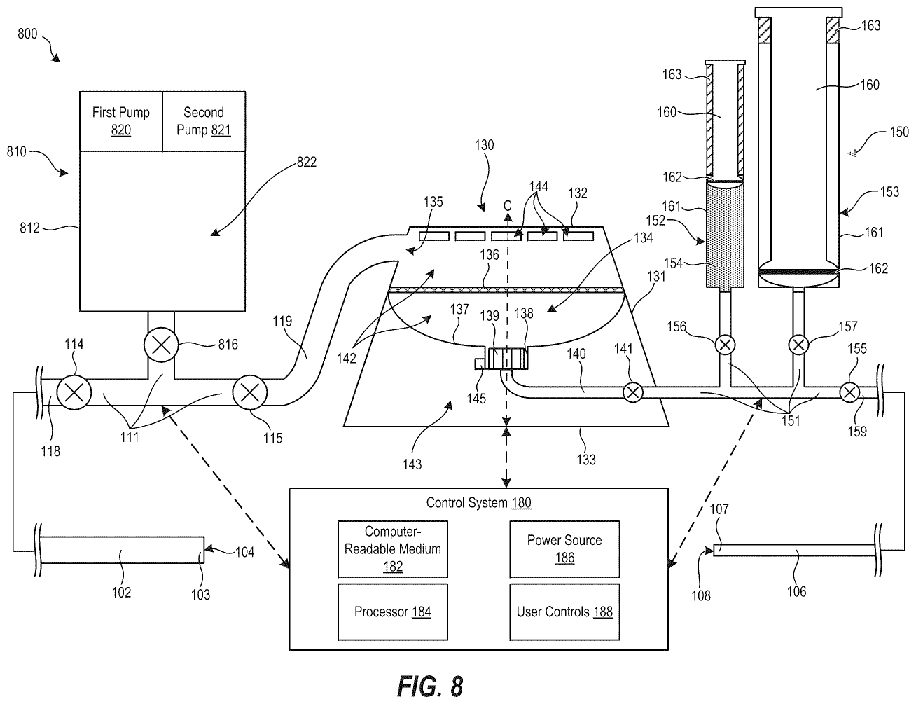

Likewise, one or more of the syringes of the system 100 can be replaced with one more vacuum pumps. FIG. 8, for example, is a partially-schematic side cross-sectional view of a clot treatment system 800 (“system 800”) in accordance with additional embodiments of the present technology. The system 100 can include several components generally similar or identical to, and can operate in a manner generally similar or identical to, the system 100 described in detail above with reference to FIGS. 1-7H. For example, similar or identical components in FIG. 8 are referred to with the same reference numbers as FIG. 1.

In the illustrated embodiment, rather than the first and second aspiration syringes 112, 113 of FIG. 1, the system 800 includes an aspiration assembly 810 having a pump assembly 812 coupled to the aspiration connection tubing 111 via a pump assembly valve 816. The pump assembly 812 can include/define a chamber 822, a first pump 820 configured to generate vacuum pressure in the chamber 822, and a second pump 821 configured to generate positive pressure in the chamber 822. While illustrated as separate pumps, the first pump 820 and the second pump 821 can comprise the same pump. For example, a single pump can have a vacuum inlet comprising the first pump 820 to generate vacuum pressure in the chamber 822 and an outlet (e.g., a positive pressure outlet) comprising the second pump 821 configured to generate positive pressure in the chamber 822. The control system 180 can control the first pump 820 to generate the vacuum pressure within the chamber 822 while the pump assembly valve 816 is closed and open the pump assembly valve 816 to apply the vacuum pressure to the aspiration lumen 104 of the aspiration catheter 102 to aspirate clot material and blood into the chamber 822. The control system 180 can control the second pump 821 to generate positive pressure to drive the clot material and blood from the chamber 822 into the filter assembly 130. In some embodiments, the chamber 822 has a volume equal to or greater than about 60 cc, about 100 cc, about 150 cc, about 200 cc, about 300 cc, about 400 cc, about 500 cc, and/or the like. In some embodiments, the control system 180 can control the first pump 820 to generate a specified level of vacuum pressure within the chamber 822 (e.g., 60 cc, 120 cc, 150 cc, etc.). In some embodiments, the first pump 820 and the second pump 821 comprise the same pump. The system 800 can include one or multiple of the pump assemblies coupled to the aspiration connection tubing 111 of the aspiration assembly 810 via corresponding pump assembly valves.

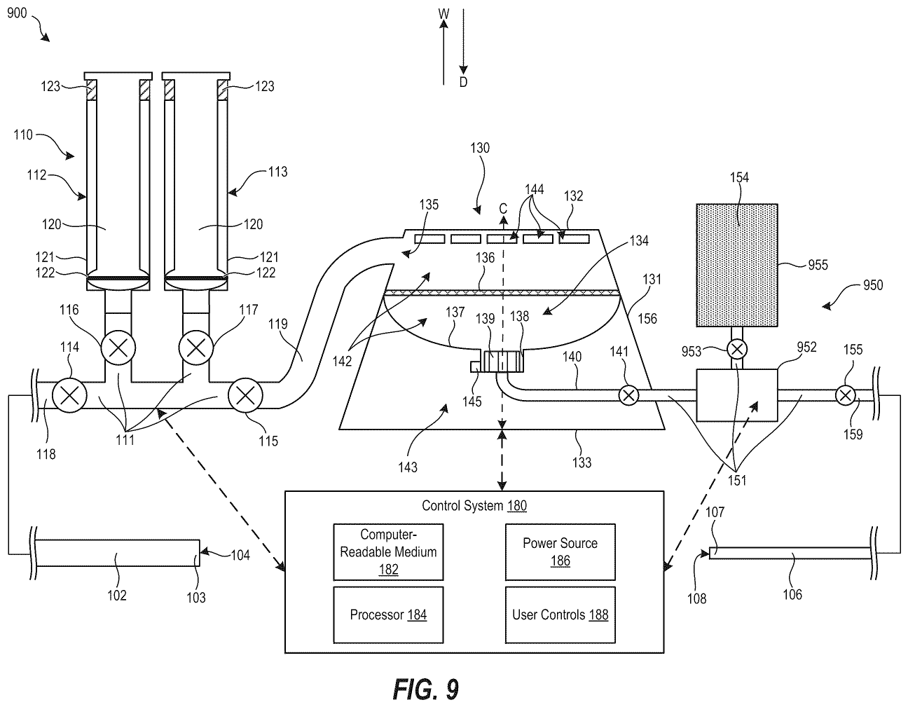

FIG. 9 is a partially-schematic side cross-sectional view of a clot treatment system 900 (“system 900”) in accordance with additional embodiments of the present technology. The system 900 can include several components generally similar or identical to, and can operate in a manner generally similar or identical to, the system 100 described in detail above with reference to FIGS. 1-7H. For example, similar or identical components in FIG. 9 are referred to with the same reference numbers as FIG. 1.

In the illustrated embodiment, rather than the flushing syringe 152 and the reinfusion syringe 153 of FIG. 1, the system 900 includes a reinfusion assembly 950 having a pump 952 coupled to the reinfusion connection tubing 151, and a reservoir 955 containing the flushing fluid 154 coupled to the pump 952 via a flushing fluid valve 953. With the filter valve 141 and the reinfusion outlet valve 155 each in an open position, the control system 180 can control the pump 952 to generate pressure to drive blood collected in the filter assembly 130 through the filter 139 and through the reinfusion connection tubing 151 to the reinfusion catheter 106. Similarly, the control system 180 can control the flushing fluid valve 953 to open and drive the pump 952 to move the flushing fluid 154 through the filter valve 141 to flush the filter 139 and/or through the reinfusion outlet valve 155 to push filtered blood through the reinfusion lumen 108 of the reinfusion catheter 106. In some embodiments, the control system 180 can control the pump 952 to pull filtered blood through the filter assembly 130 and drive the filtered blood into the reinfusion lumen 108 of the reinfusion catheter 106 when the sensor 145 indicates that the filter assembly 130 has blood therein, and cease operation of the pump 952 when the sensor 145 indicates that the level of blood in the filter assembly 130 is below a predetermined level.

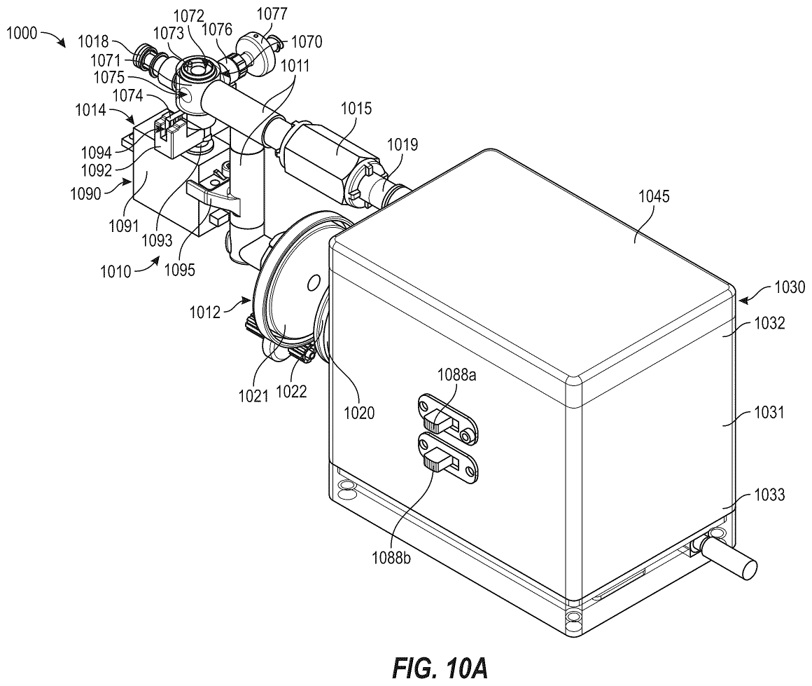

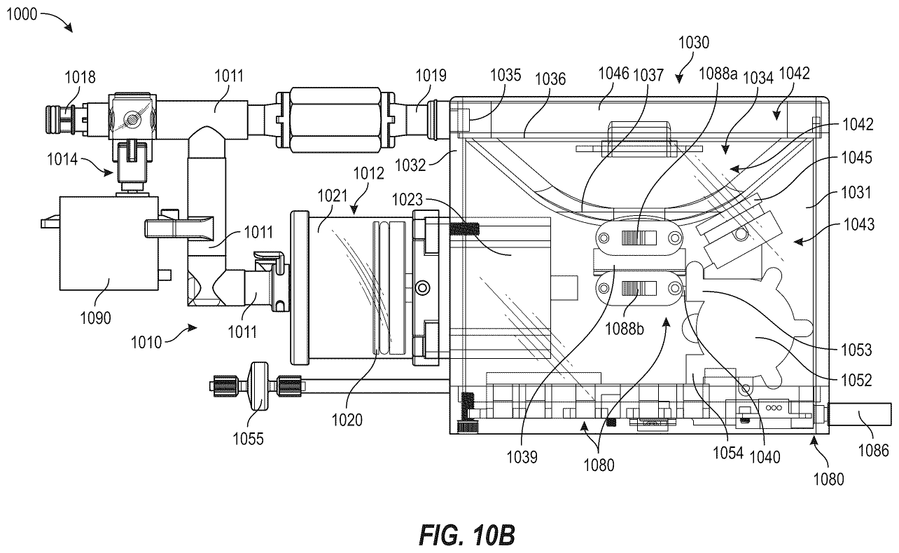

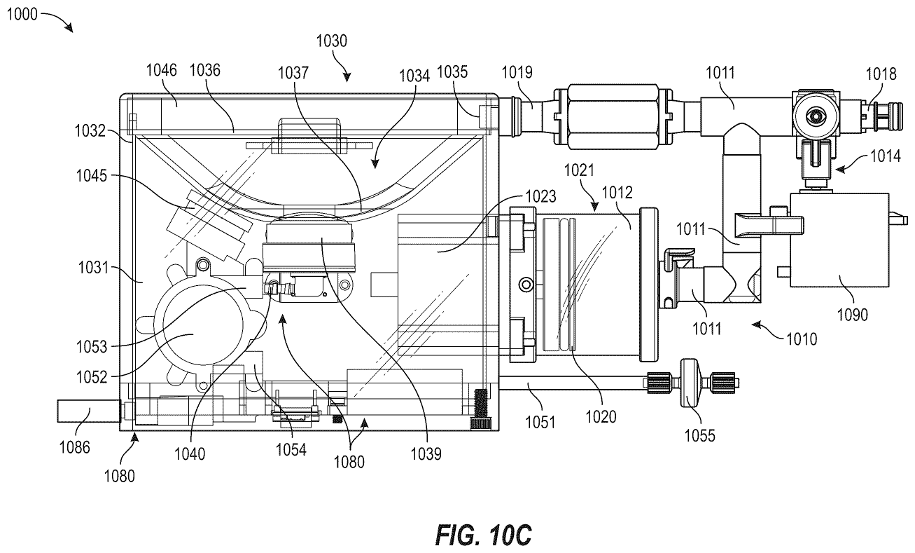

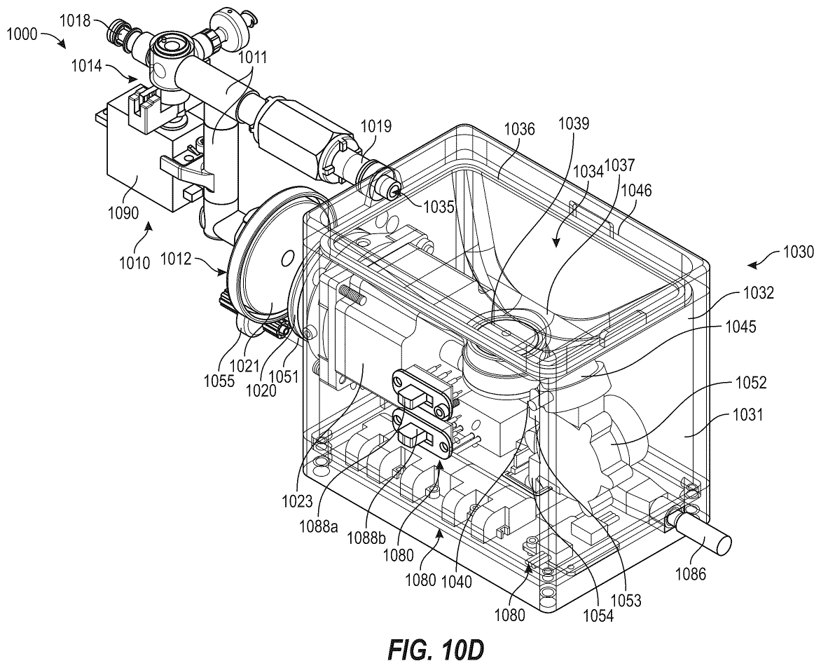

FIG. 10A is an isometric view of a clot treatment system 1000 (“system 1000”) in accordance with additional embodiments of the present technology. The system 1000 can include several components generally similar or identical to, and can operate in a manner generally similar or identical to, the system 100 and/or the system 900 described in detail above with reference to FIGS. 1-7H and 9. For example, in the illustrated embodiment the system 1000 includes an aspiration assembly 1010 selectively fluidly couplable to a filtering and reinfusion assembly 1030.

The aspiration assembly 1010 can include aspiration connection tubing 1011, an aspiration inlet 1018, an aspiration outlet 1019, an aspiration syringe 1012, an aspiration inlet valve 1014, and an aspiration outlet valve 1015. The aspiration inlet 1018 can comprise a connector, such as a large bore (e.g., 24 French, 20 French, greater than 16 French) Toomey tip connector, and is configured to be fluidly coupled to the lumen of an aspiration catheter (e.g., the aspiration lumen 104 of the aspiration catheter 102 of FIGS. 1-2B) configured to be positioned within the vasculature of a patient proximate to clot material therein. The aspiration connection tubing 111 can have a size and corresponding inner diameter equal to or greater than a size and corresponding inner diameter of the aspiration inlet 1018, such as about 16 French, about 18 French, about 20 French, about 22 French, about 22 French, about 24 French, and/or the like.

The aspiration inlet valve 1014 is configured to be controlled by a control system 1080 (FIGS. 10B-10D) of the system 100 to open and close (e.g., move between an open position and a closed position) to fluidly connect and fluidly disconnect the aspiration connection tubing 1011 from the aspiration inlet 1018. In the illustrated embodiment, for example, the aspiration inlet valve 1014 is an electromechanically controlled stopcock valve including a stopcock valve assembly 1070 operably coupled to an actuator assembly 1090 (e.g., a motor assembly, an electromechanical actuator assembly, a mechanical actuator assembly, and/or the like). The stopcock valve assembly 1070 can include (i) a base 1071 (shown as transparent in FIG. 10A) fixedly (e.g., non-movably) coupled between the aspiration connection tubing 1011 and the aspiration inlet 1018 and defining a lumen 1072 and (ii) a plunger 1073 at least partially positioned within the lumen 1072 of the base 1071 and at least partially rotatable therein. The plunger 1073 can define a lumen or through hole 1075 and further comprise one or more tabs (e.g., projections, fins) 1074 coupled to the plunger 1073 and positioned outside the lumen 1072. The through hole 1075 can have a diameter equal to or greater than about 16 French, about 18 French, about 20 French, about 22 French, about 22 French, about 24 French, and/or the like. The actuator assembly 1090 can include a motor 1091 operably coupled to an engagement member 1092 via a shaft 1093. The motor 1091 can be a servomotor and/or other type of motor configured to rotate the shaft 1093 to rotate the engagement member 1092. The engagement member 1092 is configured to engage with (e.g., mate with) the plunger 1073 such that, when the motor 1091 drives the shaft 1093 to rotate the engagement member 1092, the engagement member 1092 correspondingly drives the plunger 1073 to rotate within the base 1071. For example, the engagement member 1092 can define a channel 1094 configured to receive and secure the one or more tabs 1074 therein. In some embodiments, the actuator assembly 1090 can be fixed to a portion of the aspiration connection tubing 1011 via a mount 1095.

In operation, the actuator assembly 1090 can be controlled to operate the motor 1091 to rotate the plunger 1073 within the base 1071 (e.g., via the shaft 1093 and the engagement member 1092) to move the through hole 1075 into and out of alignment within the fluid pathways of the aspiration inlet 1018 and the aspiration connection tubing 1011 to fluidly connect the aspiration inlet 1018 to the aspiration connection tubing 1011 and to fluidly disconnect the aspiration inlet 1018 from the aspiration connection tubing 1011, respectively. In the illustrated embodiment, the aspiration inlet valve 1014 is in a closed position in which the through hole 1075 is rotated about 90 degrees relative to the adjacent lumens of the aspiration inlet 1018 and the aspiration connection tubing 1011 such that the aspiration inlet 1018 is fluidly disconnected from the aspiration connection tubing 1011. The aspiration inlet valve 1014 can be moved to an open position by controlling the motor 1091 to rotate the plunger 1073 about 90 degrees from the position shown in FIG. 10A to at least partially align the through hole 1075 with the adjacent lumens of the aspiration inlet 1018 and the aspiration connection tubing 1011 such that the aspiration inlet 1018 is fluidly connected to the aspiration connection tubing 1011. In other embodiments, the aspiration inlet valve 1014 can comprise another type of valve other than a stopcock valve (e.g., solenoid valve, pinch valve, etc.).

In some embodiments, the aspiration inlet valve 1014 can further comprise a flush port 1076 configured to provide fluid access to the aspiration inlet 1018 and/or to the aspiration connection tubing 1011 in at least some positions of the plunger 1073 (e.g., when the plunger 1073 is in the closed position illustrated in FIG. 10A). A check valve 1077 can be coupled to the flush port 1076 and configured to (i) permit fluid flow into the flush port 1076 and into the aspiration inlet 1018 and/or into the aspiration connection tubing 1011 and (ii) inhibit or even prevent fluid flow from the aspiration inlet 1018 and/or the aspiration connection tubing 1011 through the flush port 1076. During operation of the system 1000, a source of flushing fluid (e.g., saline), such as a syringe or pressure source containing the flushing fluid, can be fluidly coupled to the check valve 1077 and utilized to drive the flushing fluid through the aspiration inlet valve 1014 and into the aspiration connection tubing 1011 and/or the aspiration catheter coupled to the aspiration inlet 1018 to flush these components.

The aspiration syringe 1012 can be generally similar to the first and second aspiration syringes 112, 113 described in detail to FIG. 1 above. For example, the aspiration syringe 1012 can include a plunger 1020 slidably positioned within a barrel 1021 (shown as transparent in FIG. 10A). The plunger 1020 can include a seal 1022 (e.g., an O-ring) positioned to slidably contact and seal against an interior surface of the barrel 1021. The barrel 1021 can have a volume equal to or greater than about 30 cubic centimeters (cc), about 40 cc, about 50 cc, about 60 cc, about 80 cc, about 100 cc, about 150 cc, about 200 cc, and/or the like.

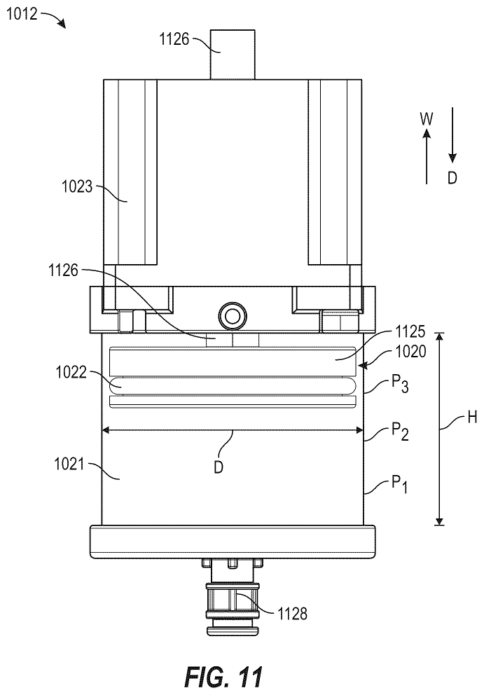

FIG. 11 is a side view of the aspiration syringe 1012 in accordance with embodiments of the present technology. Referring to FIG. 11, the aspiration syringe 1012 further includes an actuator 1023 (e.g., a motor, an electromechanical actuator, a mechanical actuator, and/or the like) configured to be controlled by the control system 1080 (FIGS. 10B-10D) of the system 1000 to (i) withdraw/retract the plunger 1020 through the barrel 1021 (e.g., in a direction W) to generate vacuum pressure within the barrel 1021 and (ii) depress/advance the plunger 1020 through the barrel 1021 (e.g., in a direction D) to expel any contents therein. More specifically, the plunger 1020 can comprise a head 1125 about which the seal 1022 is positioned, and a shaft 1126 coupled to the head 1125 and operably coupled to the actuator 1023. The actuator 1023 can operate to drive the shaft 1126 in the directions W and D to drive the head 1125 through the barrel 1021 to generate vacuum pressure and expel contents therein, respectively. In some embodiments, the shaft 1126 is threaded and the actuator 1023 comprises a motor configured to rotate the threaded shaft 1126 in a first direction to drive the plunger 1020 in the direction W and a second direction, opposite the first direction, to drive the plunger 1020 in the direction D. In some such embodiments, the actuator 1023 comprises a stepper motor configured to rotate the shaft 1126. The aspiration syringe 1012 can further comprise a tip 1128 (e.g., a Toomey tip or connector) configured to be coupled to the aspiration connection tubing 1011 (FIG. 10A). The tip 1128 can have a size and corresponding inner diameter equal to or greater than about 16 French, about 18 French, about 20 French, about 22 French, about 22 French, about 24 French, and/or the like.

In the illustrated embodiment, the barrel 1021 has a diameter A and a height H. In some embodiments, the diameter A is relatively large such that the height H can be relatively small compared to conventional syringes of the same volume. It is known that as the diameter of a syringe increases, a force required to pull vacuum within the syringe also increases. This force is a limiting factor on the volume of conventional syringes operated manually by a physician—as the force cannot be too high such that the physician cannot, or must unduly work, to withdraw the plunger to generate vacuum. In some aspects of the present technology, the actuator 1023 can be significantly more powerful than a human operator such that the actuator 1023 can withdraw the plunger 1020 in the direction W to generate vacuum pressure within the barrel 1021 even given the relatively large diameter A. In additional aspects of the present technology, increasing the diameter A can correspondingly decrease the height H of the barrel 1021 and a corresponding required length of the shaft 1126 to enable a full stroke of the plunger 1020 within the barrel 1021. This can decrease the overall footprint of the aspiration syringe 1012 enabling the aspiration syringe 1012 to occupy less space within the system 1000 (FIG. 10A). Additionally, the actuator 1023 can be powerful enough to fully withdraw the plunger 1020 even when the barrel 1021 has a large volume (e.g., greater than about 60 cc, greater than about 100 cc, greater than about 120 cc)—where manual operation of the aspiration syringe 1012 would be difficult or impossible for a human user.

In some embodiments, the actuator 1023 can be controlled (e.g., by the control system 1080 shown in FIGS. 10B-10D) to precisely withdraw the plunger 1020 through the barrel 1021 to generate a selected volume of vacuum pressure for subsequent aspiration. For example, the actuator 1023 can be controlled to withdraw the plunger 1020 in the direction W to a first position P₁ corresponding to a first vacuum volume, a second position P₂ corresponding to a second vacuum volume greater than the first vacuum volume, and a third position P₃ corresponding to a third vacuum volume greater than the second vacuum volume. Any number of discrete positions of the plunger 1020—and corresponding vacuum volumes—are possible. In some aspects of the present technology, such control of the volume of vacuum within the aspiration syringe 1012 can enable precise control of aspiration volume to, for example, allow for large aspirations to remove large clot, small aspirations that reduce blood loss, etc.

Referring to FIG. 10A, the aspiration outlet valve 1015 can comprise a check valve configured to (i) permit fluid flow from the aspiration connection tubing 1011 to the aspiration outlet 1019 and (ii) inhibit or even prevent fluid flow from the aspiration outlet 1019 to the aspiration connection tubing 1011. Accordingly, the aspiration outlet valve 1015 can be a passive valve that is not controlled by the control system 1080 (FIGS. 10B-10D). In other embodiments, the aspiration outlet valve 1015 can be an electromechanically-controlled valve.

The filtering and reinfusion assembly 1030 can include a body or housing 1031 having an upper portion 1032 (e.g., a first portion) and a lower portion 1033 (e.g., a second portion). A splashguard or lid 1046 can be releasably/movably coupled to the housing 1031. The lid 1046 is shown in a closed position in FIG. 10A. In some embodiments, the lid 1046 is pivotably coupled at one edge to the upper portion 1032 of the housing 1031. In other embodiments, the lid 1046 is coupled to the housing 1031 in a different manner (e.g., slidably coupled) or simply configured to rest on and potentially mate with (e.g., via a snap fit, friction fit) the upper portion 1032 of the housing 1031. FIGS. 10B-10D are a first side view, a second side view, and an isometric view, respectively, of the system 1000 of FIG. 10A in accordance with embodiments of the present technology. The housing 1031 and the lid 1046 are shown as partially transparent in FIGS. 10B-10D for clarity.

Referring to FIGS. 10B-10D the housing 1031 defines an interior or chamber 1034 and an opening or inlet 1035 near the upper portion 1032 and/or the lid 1046 that is fluidly coupled to the aspiration outlet 1019 of the aspiration assembly 1010 for, for example, receiving aspirated blood and clot material therethrough, as described in further detail below. The reinfusion and filtering assembly 1030 can further include a filter plate or filter tray 1036 (shown as partially transparent in FIG. 10D) spanning laterally across the chamber 1034 below the inlet 1035, and a collection component 1037 (shown as partially transparent in FIGS. 10B-10D) spanning laterally across the chamber 1034 below the filter tray 1036. In some embodiments, the collection component 1037 slopes downward in a direction toward a central axis of the reinfusion and filtering assembly 1030 toward a filter 1039 (which may be positioned in a receiving portion, chamber, hole, etc., of the collection component 1037).

A filter conduit 1040 (e.g., a filter tube, one or more tubes, barbed connectors, etc.) can be fluidly coupled downstream of the filter 1039. The filter conduit 1040 can fluidly couple the chamber 1034 to an inlet 1053 of a pump 1052 via the filter 1039. In the illustrated embodiment, a reinfusion conduit 1051 fluidly couples an outlet 1054 of the pump 1052 to a reinfusion outlet valve 1055. The pump 1052 can be a rotary pump, a centrifugal pump, and/or another type of pump configured to move fluid therethrough from the inlet 1053 to the outlet 1054. The reinfusion outlet valve 1055 can be fluidly coupled to the lumen of a reinfusion catheter (e.g., the reinfusion lumen 108 of the reinfusion catheter 106 of FIGS. 1-2B) configured to be positioned within the vasculature of a patient. The reinfusion outlet valve 1055 can comprise a check valve configured to (i) permit fluid flow from the reinfusion conduit 1051 to the reinfusion catheter coupled thereto and (ii) inhibit or even prevent fluid flow from the reinfusion catheter to the reinfusion conduit 1051. Accordingly, the reinfusion outlet valve 1055 can be a passive valve that is not controlled by the control system 1080 (FIGS. 10B-10D). In other embodiments, the reinfusion outlet valve 1055 can be an electromechanically-controlled valve.

Similar to the filter assembly 130 described in detail above with reference to FIG. 1, the filter tray 1036 can have a first porosity and the filter 1039 can have a second porosity less than the first porosity (e.g., the filter tray 1036 has larger pores than the filter 1039 to permit larger particles to pass therethrough). Likewise, in some embodiments the filter 1039 comprises multiple filter layers having different porosities. The filter 1039 can be pleated along a circumference thereof to increase a filtering surface area. In operation, the filter assembly 1030 can receive blood and clot material into the chamber 1034 through the inlet 1035 from the aspiration outlet 1019 of the aspiration assembly 1010. The blood and clot material can move (e.g., flow) downward toward/onto/through the filter tray 1036 via a gravity. The filter tray 1036 can inhibit or even prevent larger portions of the aspirated material (e.g., larger portions of the clot material, coagulated blood) from flowing therethrough while permitting smaller portions of the aspirated material (e.g., blood, smaller portions of the clot material) to move therethrough downward toward/onto the collection component 1037. Accordingly, the filter tray 1036 provides a first filter stage that filters out large portions of the clot material. The collection component 1037 can direct the first-stage filtered material toward the filter 1039 positioned within the receiving portion 1038. In some embodiments, the pump 1052 can generate negative pressure to draw the first-stage filtered material through the filter 1039, through the filter conduit 1040, into the inlet 1053 of the pump 1052, and through the pump 1052 to the reinfusion conduit 1051 via the outlet 1054 of the pump 1052. The filter 1039 can provide a second filtering stage that filters out smaller portions of clot material while permitting blood to pass therethrough. Additionally or alternatively, the first-stage filtered material can flow through the filter 1039 at least partially due to gravity. Accordingly, the filter assembly 1030 is configured to receive aspirated blood and clot material from the aspiration assembly 1010, filter the clot material from the blood, and permit the filtered blood to pass to the reinfusion conduit 1050 and into the reinfusion catheter coupled to the reinfusion outlet valve 1055. In some aspects of the present technology, the filter tray 1036 can filter out large portions of the clot material that may otherwise clog or interfere with the operation of the filter 1039.

In some embodiments, the housing 1031 and/or the lid 1046 are at least partially transparent to allow a user of the system 1000 (e.g., a surgeon and/or healthcare team member) to view clot material collected on the filter tray 1036 and/or proximate to the filter 1039. In some embodiments, the lid 1046 can be moved (e.g., removed, pivoted) to provide access to clot material collected on the filter tray 1036 and/or in the chamber 1034 for, for example, removal thereof.

In the illustrated embodiment, the collection component 1037 and/or the filter 1039 divide the chamber 1034 into an upper chamber portion 1042 and a lower chamber portion 1043. The upper chamber portion 1042 is configured to receive the aspirated blood and clot material and, in some embodiments, can have a volume greater or significantly greater than the volume of the aspiration syringe 1012. For example, the upper chamber portion 1042 can have a volume equal to or greater than about 100, about 200 cc, about 300 cc, about 400 cc, about 500 cc, about 600 cc, about 1000 cc, and/or the like. Accordingly, the upper chamber portion 1042 can be sized to store/hold material aspirated multiple times via the aspiration syringe 1012, as described in detail herein. In some embodiments, the lower chamber portion 1043 is substantially sterile and/or isolated from the aspirated material.

In the illustrated embodiment, the system 100 further comprises a control system 1080 positioned at least partially within the lower chamber portion 1043 of the housing 1031. The control system 1080 can be include features generally similar or identical to the control system 180 described in detail above with reference to FIG. 1. For example, the control system 1080 can comprise can a non-transitory computer-readable medium, a processor, a power source, and one or more user controls. These components can be secured (e.g., via screw, fasteners) to the housing 1031 within the lower chamber portion 1043 and can comprise circuit boards, power converters, wire connectors, and/or other electrical components. The control system 1080 can be operably/communicatively coupled to the various electrical components of the system 1000, such as the actuator assembly 1090 of the reinfusion inlet valve 1014, the actuator 1023 of the aspiration syringe 1012, and the pump 1052, etc., via one or more wired and/or wireless connections. Accordingly, the computer-readable medium can store instructions that, when executed by the processor, carry out the functions attributed to the control system 1080 as described herein. In the illustrated embodiment, the power source comprises an electrical cable 1086 that can, for example, be electrically coupled to a source of AC power within an operating room, such as a wall socket. In other embodiments, the power source can comprise one or more batteries and/or other power sources fully contained within the housing 1031. In some embodiments, the one or more user controls can comprise a first switch 1088a and a second switch 1088b (obscured in FIG. 10C; also shown in FIG. 10A) mounted to an external surface of the housing 1031 and accessible to a user. As described in further detail below, in some embodiments the first switch 1088a is configured to be actuated by a user to initiate one or more aspiration operations of the system 1000, and the second switch 1088b is configured to initiate one or more filtered blood reinfusion operations of the system 1000. In other embodiments, the first switch 1088a and/or the second switch 1088b can be configured as buttons, sliders, touchscreen elements, and/or the like.

In some embodiments, the system 1000 further includes a sensor 1045 (e.g., a liquid level sensor) configured to detect a level of fluid (e.g., blood) within the chamber 1034. The control system 1080 can be communicatively coupled to the sensor 1045 for receiving data about the level of fluid within the chamber 1034. The sensor 1045 can comprise a pressure sensor, a capacitance-sensor, and/or the like.

In some embodiments, some or all of the electrical components of the system 1000 are positioned/housed within the lower chamber portion 1043. For example, the control system 1080, the pump 1052, the actuator 1023 of the aspiration syringe 1012, and the sensor 1045 are positioned within the lower chamber portion 1043. As noted above, the lower chamber portion 1043 can be isolated from the upper chamber portion 1042 and sterile such that these components are not exposed to blood and/or other fluids during operation of the system 1000. The housing 1031 can provide a rigid surface for mounting of the electrical components as well as other components of the system 1000, such as the aspiration assembly 1010. In some embodiments, the system 1000 is entirely disposable and configured for use during a single clot treatment procedure. In other embodiments, the various electrical components can be reused in multiple clot treatment procedures. For example, the actuator assembly 1090 of the aspiration inlet valve 1014 can be detached from the plunger 1073 and subsequently reused. Likewise, various components not exposed to blood and/or clot material, such as the control system 1080, the actuator 1023 of the aspiration syringe 1012, the sensor 1045, etc., can be remove/detached from the housing 1031 and reused. Moreover, the system 1000 can be relatively compact and can be configured (e.g., sized, shape) to be portable.

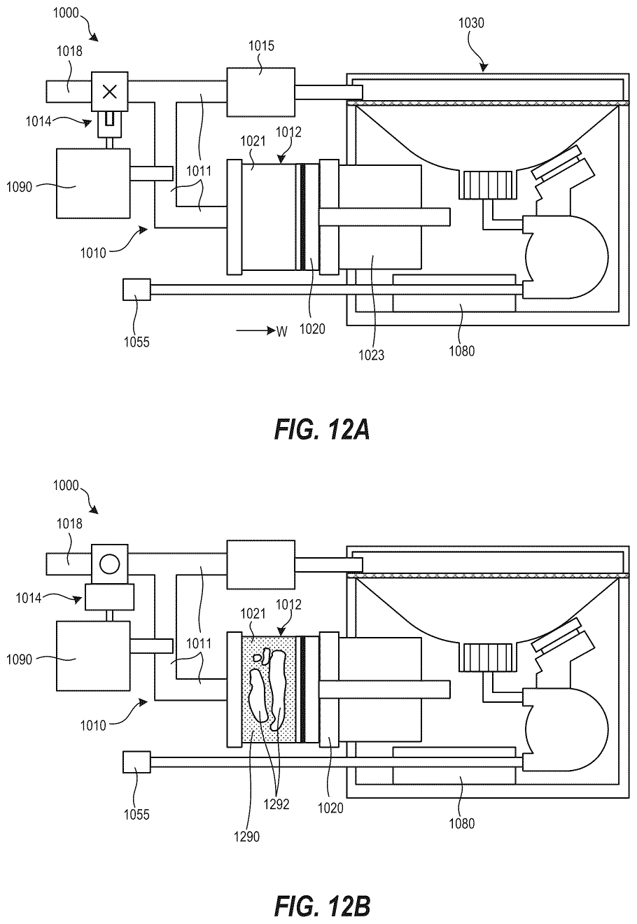

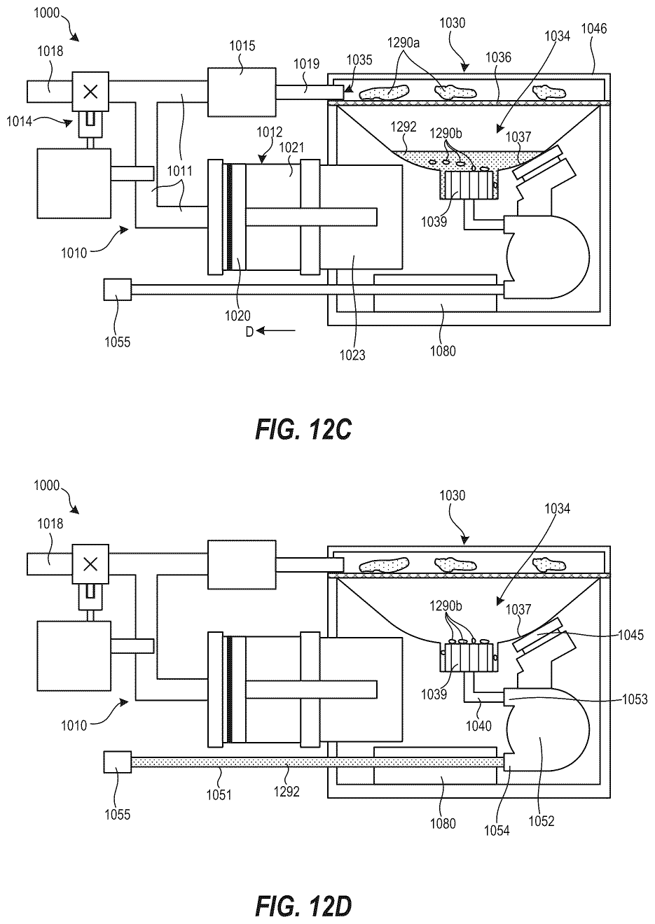

The control system 1080 can control the aspiration inlet valve 1014, the aspiration syringe 1012, and the pump 1052 in various sequences to perform a myriad of aspiration, blood filtering, filtered blood reinfusion, and/or system flushing operations during a clot removal procedure (e.g., thrombectomy procedure) carried out on a patient. Some such operations are described in detail below with reference to FIGS. 12A-12D, and can be generally similar or identical to any of the sequences/methods described herein. One of ordinary skill in the art will appreciate that the various operations may be combined and/or modified.

FIGS. 12A-12D are simplified side cross-sectional views of the clot treatment system 1000 of FIGS. 10A-10D illustrating a method or sequence performed by the system 1000 of aspirating clot material and blood from a patient, filtering the blood from the clot material, and reinfusing the filtered blood into the patient in accordance with embodiments with the present technology. Although an aspiration catheter (e.g., the aspiration catheter 102 of FIGS. 1-2B) and a reinfusion catheter (e.g., the reinfusion catheter 106 of FIGS. 1-2B) are not shown in FIGS. 12A-12D for clarity, the aspiration catheter can have a proximal portion that is fluidly coupled to the aspiration inlet 1018 and a distal end that can be positioned proximate to clot material within the vasculature of a patient, such as proximal to, within, and/or distal to the clot material within the vasculature. In some embodiments, the clot material comprises a pulmonary embolism within a pulmonary artery of the patient, a deep vein thrombosis (DVT) within a peripheral vein of the patient, and/or the like. The reinfusion catheter can likewise be positioned within the vasculature of the patient through the same or a different access site as the aspiration catheter, as described in detail above with reference to FIGS. 1-2B, and the reinfusion outlet valve 1055 can be fluidly coupled to the reinfusion catheter (e.g., to a proximal portion thereof).

Referring to FIGS. 10A, 10B, and 10D, in some embodiments to perform aspiration of the clot material, a user of the system 1000 (e.g., a surgeon and/or other healthcare team member) can actuate (e.g., slide) the first switch 1088a to trigger the control system 1080 (FIGS. 10B-10D) to perform an aspiration cycle/sequence. Referring to FIG. 12A, after actuation of the first switch 1088a, the control system 1080 can control the actuator assembly 1090 of the aspiration inlet valve 1014 to move the aspiration inlet valve 1014 to the closed position (if the aspiration inlet valve 1014 is not already in a closed position; as indicated by an “X” on the aspiration inlet valve 1014). More specifically, referring to FIG. 10A, the control system 1080 can control the actuator assembly 1090 to rotate the plunger 1073 via the shaft 1093 and the engagement member 1092 such that the through hole 1075 is out of alignment with and does not fluidly connect the aspiration inlet 1018 to the aspiration connection tubing 1011. Referring again to FIG. 12A, the control system 1080 can then control the actuator 1023 of the aspiration syringe 1012 to withdraw the plunger 1020 in the direction W through the barrel 1021 of the aspiration syringe 1012 to generate vacuum pressure (e.g., negative pressure) within the barrel 1021. Because the aspiration inlet valve 1014 is closed, vacuum is charged within the barrel 1021 of the aspiration syringe 1012 (e.g., a negative pressure is maintained) before the aspiration syringe 1012 is fluidly connected to the aspiration lumen of the aspiration catheter. Moreover, the aspiration outlet valve 1015 inhibits the negative pressure from being applied to the filtering and reinfusion assembly 1030. That is, the aspiration outlet valve 1015 inhibits or even prevents fluid flow from the reinfusion assembly 1030 to the aspiration connection tubing 1011 and the aspiration syringe 1012 when the negative pressure is generated in the aspiration syringe 1012.

Referring to FIG. 12B, the control system 1080 can then open the aspiration inlet valve 1014 (as indicated by an “0” on aspiration inlet valve 1014) to fluidly connect the barrel 1021 of the aspiration syringe 1012 to the aspiration lumen of the aspiration catheter via the aspiration inlet 1018 and the aspiration connection tubing 1011. More specifically, referring to FIG. 10A, the control system 1080 can control the actuator assembly 1090 to rotate the plunger 1073 via the shaft 1093 and the engagement member 1092 such that the through hole 1075 aligned with and fluidly connects the aspiration inlet 1018 to the aspiration connection tubing 1011. Accordingly, referring again to FIG. 12B, the vacuum stored in the barrel 1021 of the aspiration syringe 1012 is applied to the aspiration lumen of the aspiration catheter to aspirate clot material 1290 and blood 1292 through the aspiration lumen of the aspiration catheter, through the aspiration inlet 1018, through the aspiration inlet valve 1014, through the aspiration connection tubing 1011, and into the barrel 1021 of the aspiration syringe 1012. In some embodiments, opening the aspiration inlet valve 1014 instantaneously or nearly instantaneously applies the stored vacuum pressure to the aspiration lumen of the aspiration catheter, thereby generating a suction pulse throughout the aspiration lumen that can aspirate the clot material 1290 and the blood 1292 into and through the aspiration lumen. In other embodiments, the aspiration inlet valve 1014 can be opened before the plunger 1020 of the aspiration syringe 1012 is withdrawn to generate vacuum pressure such that the clot material 1290 and the blood 1292 are aspirated continuously as the vacuum pressure is generated—that is, for example, without or substantially without storing the vacuum pressure in the barrel 1021 of the aspiration syringe 1012.

In some aspects of the present technology, the aspiration syringe 1012 is no longer under vacuum after opening the aspiration inlet valve 1014. Accordingly, the blood collected in the barrel 1021 is not held under vacuum therein, which can inhibit or even prevent damage to the blood—for example, as compared to systems that store blood under vacuum such that it may boil or otherwise damagingly increase in temperature due to the reduced boiling point caused by the reduced pressure.

Referring to FIGS. 10A and 12B, in additional aspects of the present technology, utilizing a stopcock valve, such as a large bore stopcock valve, for the aspiration inlet valve 1014 can improve the efficiency of clot aspiration. In particular, the actuator assembly 1090 can enable the stopcock valve assembly 1070 to be quickly moved from a fully closed position to a fully open position, which allows the vacuum pressure stored in the barrel 1021 of the aspiration syringe 1012 to be applied to the aspiration catheter in a quick burst or pulse that is expected to improve the aspiration of the clot material 1290. Moreover, the flow path through the through hole 1075 of the stopcock valve assembly 1070 is unrestricted in the open position—providing a straight and large lumen through the aspiration inlet valve 1014—which is again expected to improve the aspiration of the clot material 1290. In contrast, other types of valves may not be able to move quickly from a fully open position to a fully closed position, and may have restricted and/or tortuous flow paths therethrough. For example, the fluid pathway through solenoid valve is typically not straight. Likewise, pinch valves require a tube of the right flexibility to close and not collapse under vacuum pressure. Such characteristics are difficult to achieve in large-bore systems such as the system 1000 including tubing sizes and flow paths of, for example, 16 French or greater, 20 French or greater, 24 French or greater, etc. Additionally, pinch valves—especially in the large bore context—may take a greater amount of time for the pinched tube to expand to a fully open position, reducing aspiration efficacy.

Referring to FIG. 12C, the control system 1080 can then (i) close the aspiration inlet valve 1014 to fluidly disconnect the aspiration syringe 1012 from the aspiration inlet 1018 and the aspiration catheter and subsequently (ii) actuate the actuator 1023 of the aspiration syringe 1012 to depress the plunger 1020 through the barrel 1021 of the aspiration syringe 1012 in the direction D to drive the clot material 1290 (identified as larger portions of clot material 1290a and smaller portions of clot material 1290b) and the blood 1292 through the aspiration connection tubing 1011, through the aspiration outlet valve 1015, through the aspiration outlet 1019, and into the chamber 1034 of the filtering and reinfusion assembly 1030. The clot material 1290 and the blood 1292 are received through the inlet 1035 of the filtering and reinfusion assembly 1030, and gravity causes the clot material 1290 and the blood 1292 to move downward through the chamber 1034 toward the filter tray 1036 and the collection component 1037. As described in detail above with reference to FIGS. 10B-10D, the filter tray 1036 can filter larger portions 1290a of the clot material 1290 while allowing the blood 1292 and smaller portions 1290b of the clot material 1290 to pass therethrough. The blood 1292 and the smaller portions 1290b of the clot material 1290 can collect on the collection component 1037 and/or around the filter 1039. In some embodiments, the user can open the lid 1046 to provide access to the filter tray 1036 and the clot material 1290a thereon for removal/cleaning.

In some embodiments, at this stage, the aspiration cycle/sequence triggered by actuation of the first switch 1088a (FIGS. 10A, 10B, and 10D) is complete. Accordingly, the user can either (i) again actuate the first switch 1088a to initiate another aspiration sequence or (ii) actuate the second switch 1088b (FIGS. 10A, 10B, and 10D) to initiate a reinfusion cycle/sequence. If the user again actuates the first switch 1088a, the system 100 can perform the operations/steps illustrated in and described in detail with reference to FIGS. 12A-12C to aspirate additional blood and clot material into the aspiration assembly 1010 and then drive the additional blood and clot material from the aspiration assembly 1010 into the filtering and reinfusion assembly 1030. As described in detail above, the chamber 1034 can be sized to receive and store clot material and blood from multiple aspiration passes utilizing the aspiration syringe 1012. That is, a volume of the chamber 1034 can be larger (e.g., two, three, four, five, ten, or more times larger) than a volume of the aspiration syringe 1012.

Upon actuation (e.g., sliding, pressing) of the second switch 1088b, the control system 1088 can trigger the control system 1080 to perform a reinfusion cycle/sequence. For example, referring to FIG. 12D, the control system 1080 can actuate the pump 1052 to (i) pull the blood 1292 through the filter 1039, through the filter conduit 1040, and into the inlet 1053 of the pump 1052 and (ii) drive/push the blood 1292 through the pump 1052, out of the outlet 1054 of the pump 1052, through the reinfusion conduit 1051, through the reinfusion outlet valve 1055, and at least partially through the reinfusion catheter fluidly coupled to the reinfusion outlet valve 1055 into the vasculature of the patient. As described in detail above with reference to FIGS. 10B-10D, the filter 1039 filters out the smaller portions 1290b of the clot material 1290 from the blood 1292 such that the blood 1292 is suitable for reinfusion into the patient. In some embodiments, the control system 1080 can control the pump 1052 to operate to reinfuse filtered blood through the reinfusion conduit 1051 only when the sensor 1045 indicates that the chamber 1034 has a predetermined level of the blood 1292 therein, and cease operation of the pump 1052 when the sensor 1045 indicates that the level of blood in the chamber 1034 is below the predetermined level. In this manner, the system 1000 can inhibit or even prevent the pump 1052 from drawing air from the chamber 1034 and, potentially, driving the air through the reinfusion conduit 1051 into the vasculature of the patient. In other embodiments, the control system 1080 can actuate the pump 1052 to automatically reinfuse blood through the reinfusion conduit 1051 whenever the sensor 1045 indicates that the level of blood in the chamber 1034 is at or above the predetermined level. In some such embodiments, this mode of operation can be caused when the second switch 1088b is actuated and remains actuated during some or all of the clot removal procedure using the system 1000.

Referring to FIGS. 10A-12D, the first and second switches 1088a, 1088b can be repeatedly actuated in various sequences to provide for one or multiple aspiration and/or reinfusion sequences. Additionally, the system 1000 can be flushed with a flushing fluid by, for example, injecting the flushing fluid through the flush port 1076 of the aspiration inlet valve 1014. With a flushing fluid in the aspiration assembly 1010, the control system 1080 can actuate the aspiration syringe 1012 to drive the flushing fluid into the filtering and reinfusion assembly 1030. The control system 1080 can then control the pump 1052 to pull the flushing fluid through the filter 1039 to flush the filter 1039 and/or to drive the flushing fluid through the reinfusion conduit 1051 and into the reinfusion catheter to drive any remaining portion of filtered blood into the vasculature of the patient.

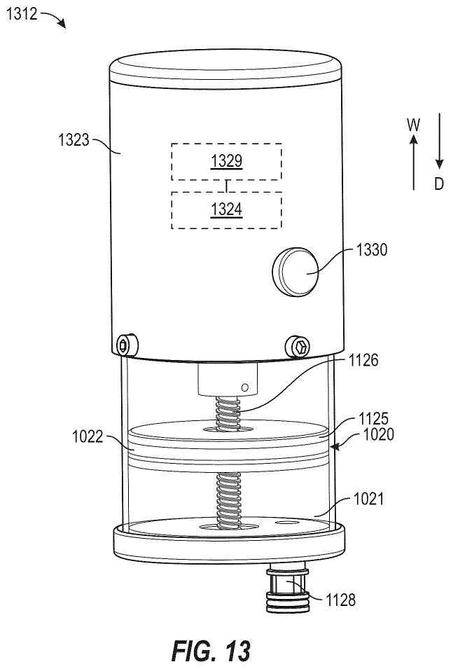

FIG. 13 is a partially-schematic side view of an aspiration syringe 1312 in accordance with additional embodiments of the present technology. The aspiration syringe 1312 can be utilized in any of the systems 100, 800, 900, and/or 1000 described in detail herein, independently, and/or in other systems, and can include several components generally similar or identical to, and can operate in a manner generally similar or identical to, the aspiration syringe 1012 described in detail above with reference to FIGS. 10A-12D. For example, in the illustrated embodiment the aspiration syringe 1312 includes the barrel 1301, an actuator 1323, the plunger 1020 configured to move through the barrel 1021, and the shaft 1126 operably coupling the plunger 1020 to the actuator 1323. The plunger 1020 can include the head 1125 having the seal 1022, and can be engaged via a threaded coupling with the shaft 1126. In some embodiments, the actuator 1323 includes a motor 1324 (shown schematically) configured to rotate the shaft 1126 and a battery 1329 (shown schematically) or other power source configured to power the motor 1324. The aspiration syringe 1312 can further include the tip 1128.

In operation, the motor 1324 is configured to (i) drive the shaft 1126 to rotate in a first direction to withdraw/retract the plunger 1020 through the barrel 1021 (e.g., in a direction W) to generate vacuum pressure within the barrel 1021 and (ii) drive the shaft 1326 to rotate in a second direction opposite the first direction to depress/advance the plunger 1320 through the barrel 1021 (e.g., in a direction D) to, for example, expel any contents therein. In some embodiments, movement of the plunger 1020 can be controlled via an actuator 1330, such as a button that can be pressed by a user to withdraw/depress of the plunger 1020 through the barrel 1021. In some aspects of the present technology, the aspiration syringe 1312 can be handheld, mobile/portable, and sized to be operated by one-hand of a user. More specifically, the battery 1324 allows the aspiration syringe 1312 to be self-contained and portable. In other embodiments, the aspiration syringe 1312 can also be coupled to a primary power supply, such as the electrical cable 1086 (FIGS. 10B-10D), and can include the battery 1324 as a back-up or alternate power supply.

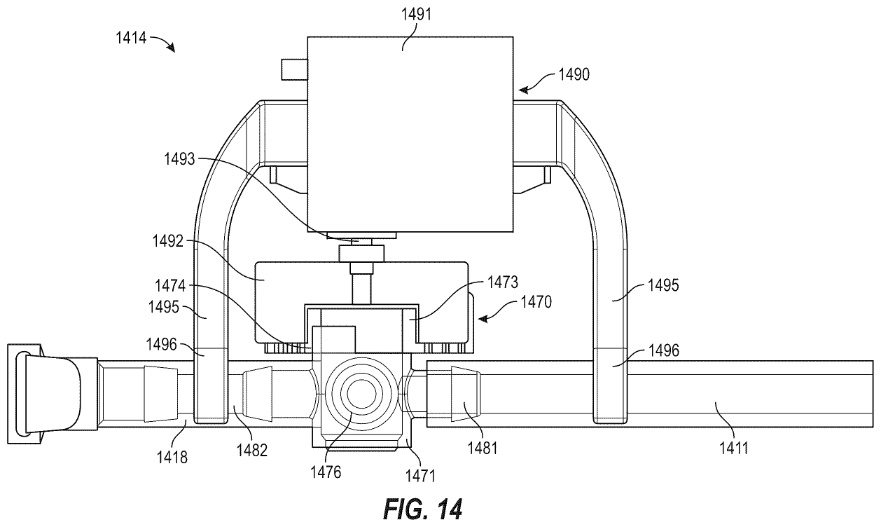

FIG. 14 is a side view of an automated stopcock valve 1414 in accordance with embodiments of the present technology. The automated stopcock valve 1414 can be utilized in any of the systems 100, 800, 900, and/or 1000 described in detail herein (e.g., as an electromechanically controlled valve) and can include several components generally similar or identical to, and can operate in a manner generally similar or identical to, the aspiration inlet valve 1014 described in detail above with reference to FIG. 10A.

For example, in the illustrated embodiment the automated stopcock valve 1414 includes a stopcock valve assembly 1470 operably coupled to an actuator assembly 1490 (e.g., a motor assembly, an electromechanical actuator assembly, a mechanical actuator assembly, and/or the like). The stopcock valve assembly 1470 can include a base 1471 (shown as transparent in FIG. 14) having a first fluid connector 1481 and a second fluid connector 1482 and defining a lumen (obscured in FIG. 14) extending therebetween. The base 1471 can be coupled between a first tube 1411 (e.g., the aspiration connection tubing 1011 of FIG. 10A) and a second tube 1418 (e.g., the aspiration inlet 1018 of FIG. 10A). The stopcock valve assembly 1470 can further include a plunger 1473 at least partially positioned within the lumen of the base 1471 and at least partially rotatable therein. The plunger 1473 can define a lumen or through hole (obscured) and further comprise one or more tabs (e.g., projections, fins) 1474 coupled to the plunger 1473 and positioned outside the base 1471. The through hole of the plunger 1473 can have a diameter equal to or greater than about 16 French, about 18 French, about 20 French, about 22 French, about 22 French, about 24 French, and/or the like. The actuator assembly 1490 can include a motor 1491 operably coupled to an engagement member 1492 via a shaft 1493. The motor 1491 can be a servomotor and/or other type of motor configured to rotate the shaft 1493 to rotate the engagement member 1492. The engagement member 1492 is configured to engage with (e.g., mate with) the plunger 1473 such that, when the motor 1491 drives the shaft 1493 to rotate the engagement member 1492, the engagement member 1492 correspondingly drives the plunger 1473 to rotate within the base 1471. For example, the engagement member 1492 can define a channel configured to receive the one or more tabs 1474 therein. In some embodiments, the automated stopcock valve 1414 can further comprise a flush port 1476 configured to provide fluid access to the first tube 1411 via the first fluid connector 1481 and/or to the second tube 1418 via the second fluid connector 1482.

In the illustrated embodiment, the actuator assembly 1490 includes a pair of arms or mounts 1495 each having a securement feature 1496 at end thereof. The securement features 1496 can each be snapped to or otherwise secured to a corresponding one of the first and second tubes 1411, 1418 to secure the actuator assembly 1490 in position relative to the stopcock valve assembly 1470. Accordingly, in some aspects of the present technology the actuator assembly 1490 can be secured to a preexisting stopcock valve assembly for motorized control thereof without modification of the stopcock valve assembly. For example, in some embodiments the stopcock valve assembly 1470 comprises a stopcock of any of the types described in U.S. patent application Ser. No. 18/182,966, titled “FLUID CONTROL DEVICES FOR CLOT TREATMENT SYSTEMS, AND ASSOCIATED SYSTEMS AND METHODS,” and filed Aug. 22, 2024, which is incorporated by reference herein in its entirety.

In operation, the actuator assembly 1490 can be controlled to operate the motor 1491 to rotate the plunger 1473 within the base 1471 (e.g., via the shaft 1493 and the engagement member 1492) to move the through hole of the plunger 1473 into and out of alignment within the first and second fluid connectors 1481, 1482 to fluidly connect the first tube 1411 to the second tube 1418 and fluidly disconnect the first tube 1411 from the second tube 1418, respectively. In some embodiments, the motor 1491 is a servomotor that can rotate the shaft 1493 by a maximum of about 90 degrees (e.g., incrementally or back and forth about a 90 degrees arc/range). Accordingly, the motor 1491 can rotate the through hole of the plunger 1473 by about 90 degrees relative to the first and second fluid connectors 1481, 1482 to bring the through hole into and out of alignment with the first and second fluid connectors 1481, 1482 to block or provide a fluid path therethrough.

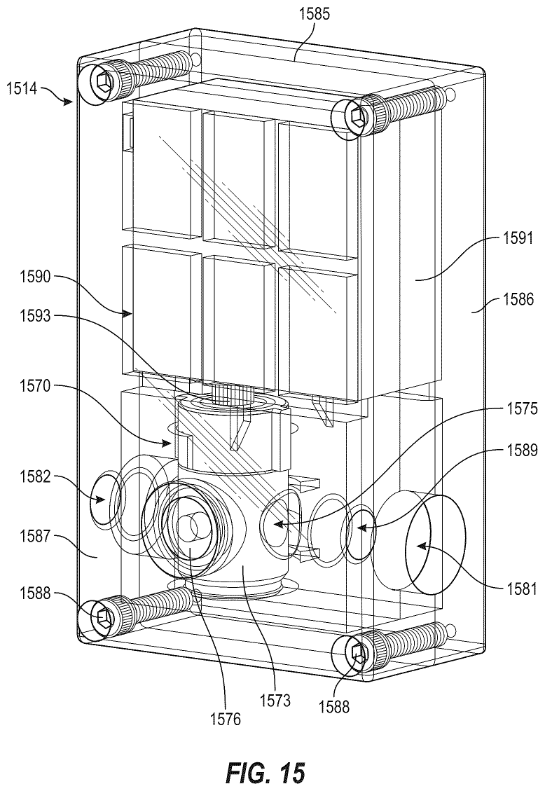

FIG. 15 is a perspective view of an automated stopcock valve 1514 in accordance with additional embodiments of the present technology. The automated stopcock valve 1414 can be utilized in any of the systems 100, 800, 900, and/or 1000 described in detail herein (e.g., as an electromechanically controlled valve) and can include several components generally similar or identical to, and can operate in a manner generally similar or identical to, the aspiration inlet valve 1014 and the automated stopcock valve 1414 described in detail above with reference to FIGS. 10A and 14, respectively.

In the illustrated embodiment, the automated stopcock valve 1514 includes a stopcock valve assembly 1570 operably coupled to an actuator assembly 1590 (e.g., a motor assembly, an electromechanical actuator assembly, a mechanical actuator assembly, and/or the like). The stopcock valve assembly 1570 and the actuator assembly 1590 are positioned/secured within a housing 1585 (e.g., between a first housing portion 1586 and a second housing portion 1587 secured together via fasteners, such as screws 1588). The housing 1585 is shown as partially transparent in FIG. 15 for clarity. The housing 1585 can define a lumen 1589 extending therethrough and having an inlet 1582 and an outlet 1581. The stopcock valve assembly 1570 can include a plunger 1573 at least partially positioned within the lumen 1589 and at least partially rotatable therein. The plunger 1573 can define a lumen or through hole 1575. The through hole 1575 and the lumen 1589 can have a diameter equal to or greater than about 16 French, about 18 French, about 20 French, about 22 French, about 22 French, about 24 French, and/or the like. The actuator assembly 1590 can include a motor 1591 operably coupled to a shaft 1593. The shaft 1593 can be directly coupled to (e.g., fixed to) the plunger 1573. The motor 1591 can be a servomotor and/or other type of motor configured to rotate the shaft 1593 to rotate the plunger 1573. In some embodiments, the automated stopcock valve 1514 can further comprise a flush port 1576 configured to provide fluid access through the plunger 1573 to either or both of the inlet 1582 and the outlet 1581 of the lumen 1589.

In operation, the actuator assembly 1590 can be controlled to operate the motor 1591 to rotate the plunger 1573 within the housing 1585 (e.g., via the shaft 1593) to move the through hole 1575 into and out of alignment with the lumen 1589 to fluidly connect the inlet 1582 to the outlet 1581 and fluidly disconnect the inlet 1582 from the outlet 1581, respectively. In some embodiments, the motor 1591 is a servomotor that can rotate the shaft 1593 by a maximum of about 90 degrees (e.g., incrementally or back and forth about a 90 degrees arc/range). Accordingly, the motor 1591 can rotate the through hole 1575 by about 90 degrees relative to the inlet 1582 and the outlet 1581 to bring the through hole into and out of alignment with the inlet 1582 and the outlet 1581 to block or provide a fluid path through the lumen 1589.

In some aspects of the present technology, the automated stopcock valve 1514 is a fully integrated unit. That is, for example, the automated stopcock valve 1514 can replace conventional stopcocks having features for manual operation, such as one or more tabs configured to be grasped and rotated by a user to open and close the stopcock.

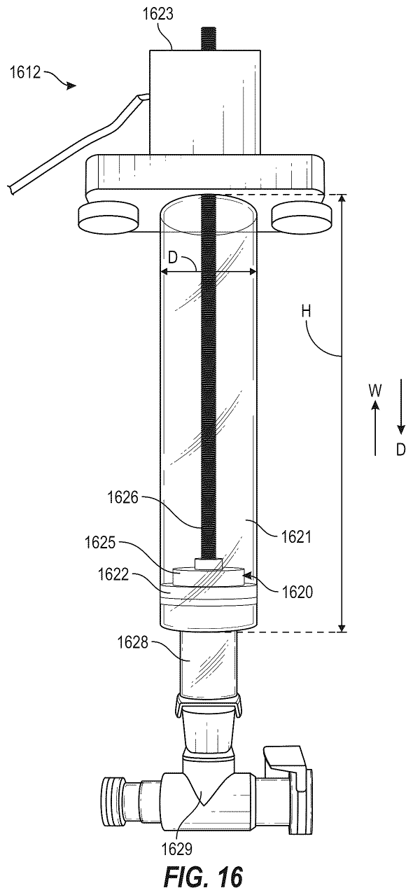

FIG. 16 is a perspective view of an automated syringe 1612 in accordance with embodiments of the present technology. The automated syringe 1612 can be utilized in any of the systems 100, 800, 900, and/or 1000 described in detail herein (e.g., as an electromechanically controlled syringe for aspiration, reinfusion, and/or flushing) and can include several components generally similar or identical to, and can operate in a manner generally similar or identical to any or all of the syringes 112, 113, 152, 153, and/or 1012 described in detail above with reference to FIGS. 1-11.

For example, the automated syringe 1612 can include a plunger 1620 slidably positioned within a barrel 1621 (shown as partially transparent in FIG. 16). The plunger 1620 can include a seal 1622 (e.g., an O-ring) positioned to slidably contact and seal against an interior surface of the barrel 1621. The barrel 1621 can have a volume equal to or greater than about 30 cubic centimeters (cc), about 40 cc, about 50 cc, about 60 cc, about 80 cc, about 150 cc, about 150 cc, about 200 cc, and/or the like. The automated syringe 1612 further includes an actuator 1623 (e.g., a motor, an electromechanical actuator, a mechanical actuator, and/or the like) configured to be controlled by the a control system to (i) withdraw/retract the plunger 1620 through the barrel 1621 (e.g., in a direction W) to generate vacuum pressure within the barrel 1621 and (ii) depress/advance the plunger 1620 through the barrel 1621 (e.g., in a direction D) to expel any contents therein. More specifically, the plunger 1620 can comprise a head 1625 about which the seal 1622 is positioned, and a shaft 1626 coupled to the head 1625 and operably coupled to the actuator 1623. The actuator 1623 can operate to drive the shaft 1626 in the directions W and D to drive the head 1625 through the barrel 1621 to generate vacuum pressure and expel contents therein, respectively. In some embodiments, the shaft 1626 is threaded and the actuator 1623 comprises a motor configured to rotate the threaded shaft 1626 in a first direction to drive the plunger 1620 in the direction W and a second direction, opposite the first direction, to drive the plunger 1620 in the direction D. In some such embodiments, the actuator 1623 comprises a stepper motor configured to rotate the shaft 1626. The automated syringe 1612 can further comprises a tip 1628 (e.g., a Toomey tip or connector) configured to be coupled to a fluid connector or tube 1629 (e.g., aspiration connection tubing, reinfusion connection tubing, etc.). The tip 1628 can have a size and corresponding inner diameter equal to or greater than about 16 French, about 18 French, about 20 French, about 22 French, about 22 French, about 24 French, and/or the like.

In the illustrated embodiment, the barrel 1621 has a diameter A and a height H. As described in detail above with reference to FIG. 11, in some embodiments the diameter A can be relatively large such that the height H can be relatively small compared to conventional syringes. In the illustrated embodiment, the diameter A and the height H of the barrel 1621 are smaller and larger, respectively, than the corresponding diameter A and height H, respectively, of the aspiration syringe 1012 of FIG. 11—more closely matching the dimensions of a conventional syringe. In some embodiments, the actuator 1623 can be controlled to precisely withdraw the plunger 1620 through the barrel 1621 to generate a selected volume of vacuum pressure or volume of fluid withdrawal (e.g., for reinfusion).

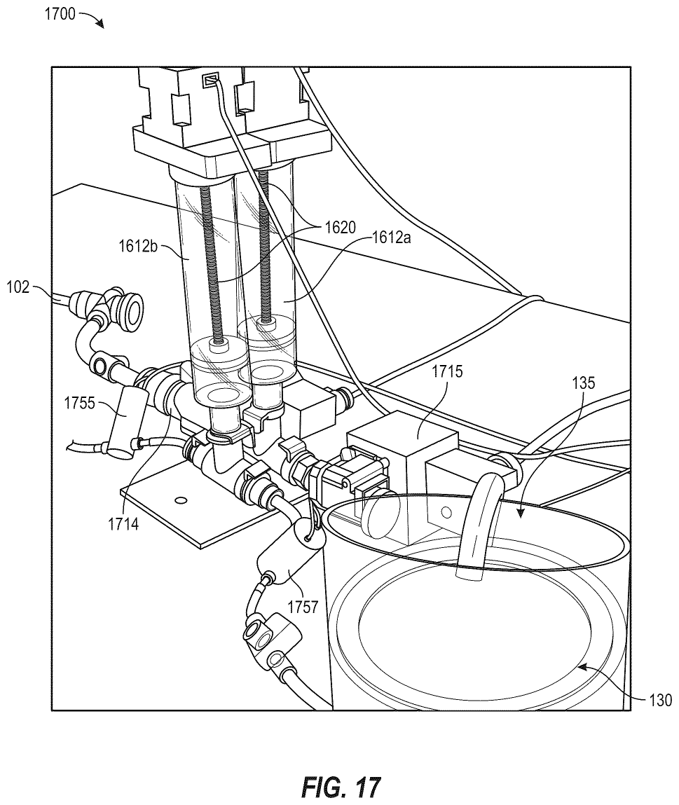

FIG. 17 is a perspective view of a clot treatment system 1700 including a pair of the automated syringes 1612 (identified individually as an automated aspiration syringe 1612a and an automated reinfusion syringe 1612b) of FIG. 16 in accordance with embodiments of the present technology. The system 1700 is generally configured as described in detail above with reference to FIGS. 1-7H. For example, in the illustrated embodiment the aspiration syringe 1612a is selectively fluidly coupled (i) to the aspiration catheter 102 via an automated aspiration valve 1714 and (ii) to the filter assembly 130 (e.g., the inlet 135 thereof) via an automated aspiration outlet valve 1715. The aspiration inlet valve 1714 and the aspiration outlet valve 1715 can be, for example, automated stopcock valves of the type described in detail above with reference to FIG. 15. In the illustrated embodiment, the reinfusion syringe 1612b is selectively fluidly coupled to (i) the filter assembly 130 via an automated reinfusion syringe valve 1757 and (ii) to a reinfusion catheter, syringe, and/or other blood receptacle (not shown) via an automated reinfusion outlet valve 1755. The reinfusion syringe valve 1757 and the reinfusion outlet valve 1755 can be, for example, automated stopcock valves of the type described in detail above with reference to FIG. 15.

As described in detail above, the system 1700 (e.g., a control system thereof, such as the control system 180 of FIG. 1) can charge vacuum in the aspiration syringe 1612a by driving the plunger 1620 of the aspiration syringe 1612a to withdraw while both the aspiration inlet valve 1714 and the aspiration outlet valve 1715 are closed. The system 1700 can then open the aspiration inlet valve 1714 to apply the stored vacuum to the catheter 102 to aspirate blood and clot material through the catheter 102 and into the aspiration syringe 1612a. The system 1700 can next drive the aspirated blood and clot material into the filter assembly 130 by driving the plunger 1620 of the aspiration syringe 1612a to depress while the aspiration inlet valve 1714 is closed and the aspiration outlet valve 1715 is open. The filter assembly 130 is configured to filter the blood from the clot material. Next, the system 1700 can draw the filtered blood into the reinfusion syringe 1612b by driving the plunger 1620 of the reinfusion syringe 1612b to withdraw while the reinfusion syringe valve 1757 is open and the reinfusion outlet valve 1755 is closed. Finally, the system 1700 can drive the filtered blood back into the patient through a reinfusion catheter or into a syringe or other blood receptacle by driving the plunger 1620 of the reinfusion syringe 1612b to depress while the reinfusion syringe valve 1757 is closed and the reinfusion outlet valve 1755 is open.

In the illustrated embodiment, the automated syringe 1712 is fluidly coupled to (i) a catheter 1703 via an automated stopcock 1714a and (ii) a filtering assembly 1730 via an automated stopcock 1714b. The automated syringe 1753 is fluidly coupled to (i) the filtering assembly 1730 and (ii) the catheter 1703. The system 1700 can include several components generally similar or identical to, and can operate in a manner generally similar or identical to, the system 100 described in detail above with reference to FIGS. 1-7H.

In some embodiments, any of the syringes (e.g., aspiration syringes, reinfusion syringes, flushing syringes, and/or the like) described herein can comprise a pneumatically-actuated syringe such as, for example, any of the syringes described in U.S. Provisional Patent Application No. 63/763,433, titled “PNEUMATIC SYRINGES, SUCH AS FOR CLOT ASPIRATION, AND ASSOCIATED SYSTEMS AND METHODS,” and filed Feb. 26, 2025, which is incorporated herein by reference in its entirety.

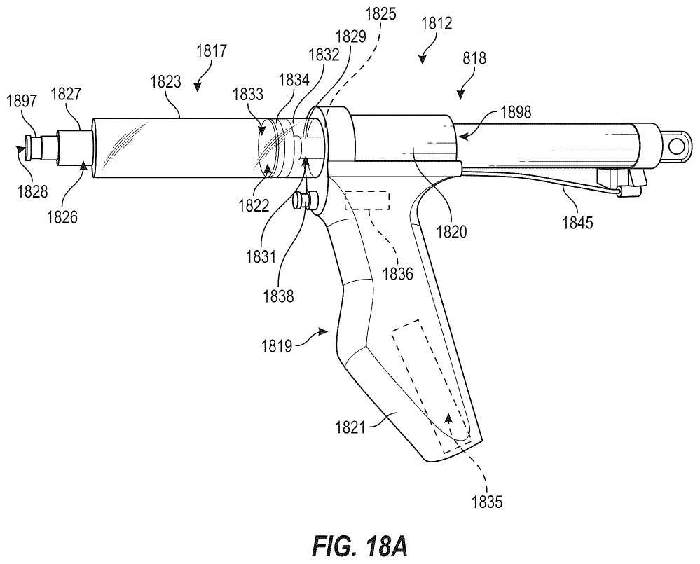

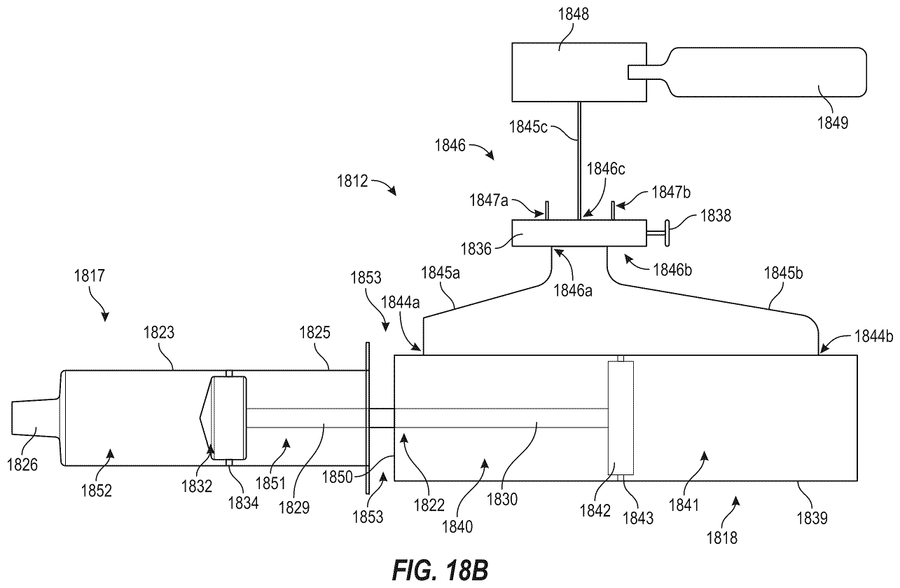

For example, FIGS. 18A and 18B are a partially-schematic side view and a side-cross-sectional view, respectively, of a pneumatic syringe 1812 in accordance with embodiments of the present technology. Referring to FIG. 18A, in the illustrated embodiment the pneumatic syringe 1812 includes a syringe 1817 and a pneumatic cylinder 1818 both coupled to a housing 1819. The housing 1819 can include a coupling portion 1820 and a storage portion 1821 (e.g., a base). In some embodiments, the housing 1819 can be configured (e.g., shaped, sized) as a handle with, for example, the storage portion 1821 configured to be gripped by a user. In other embodiments, the housing 1819 can have other configurations. The housing 1819 can define a lumen 1898 extending through the coupling portion 1820. The syringe 1817 and the pneumatic cylinder 1818 can be positioned/secured partially or completely within the lumen 1898 and coupled to the coupling portion 1820 of the housing 1819. In the illustrated embodiment, the syringe 1817 extends distally from and generally perpendicular to the housing 1819 from the lumen 1898 of the coupling portion 1820 and the pneumatic cylinder 1818 extends proximally and generally perpendicular to the handle 1819 from the lumen 1898 of the coupling portion 1820.

In the illustrated embodiment, the syringe 1817 includes a plunger assembly 1822 slidably positioned within a barrel 1823. The barrel 1823 can be generally cylindrical and can extend between a proximal end portion 1825 (obscured in FIG. 18A; e.g., a proximal flange) and a distal tip 1826 (e.g., a distal end portion). The proximal end portion 1825 can be secured to the coupling portion 1820 of the housing 1819 within the lumen 1898 and/or can be secured to the pneumatic cylinder 1818. In some embodiments, the barrel 1823 has a volume of about 60 cc or greater than about 60 cc. The distal tip 1826 can be releasably or permanently coupled to an adaptor 1827. In some embodiments, the adaptor 1827 can define a bore 1828 having a size (e.g., and corresponding inner diameter) equal to or greater than 16 Fr, 18 Fr, 20 Fr, 22 Fr, 24 Fr, 26 Fr, 28 Fr, 30 Fr, 32 Fr, and/or the like. In the illustrated embodiment, the adaptor 1827 is a Toomey-tip adaptor having a sealing member 1897 (e.g., an O-ring) extending around an exterior surface thereof for sealingly engaging (e.g., connecting to) a Toomey fitting or Toomey adaptor. In other embodiments, the adaptor 1827 can be omitted and the distal tip 1826 of the barrel 1823 can be directly coupled to a suitable connector, and/or the adaptor 1827 can be another type of adaptor such as, for example, a Luer lock, Lock slip, and/or needle.

The plunger assembly 1822 includes a shaft 1829 (partially obscured in FIG. 18B; having a proximal end portion 1830 (FIG. 3) and a distal end portion 1831). The distal end portion 1831 of the shaft 1829 can be coupled to (e.g., integrally formed, releasably, or permanently attached to) a sealing head 1832. In the illustrated embodiment, the sealing head 1832 has an annular shape including a circumferential groove 1833. The circumferential groove 1833 in the sealing head 1832 can receive a sealing member 1834 therein, such as an O-ring. The sealing head 1832 is configured to sealingly engage an interior surface of the barrel portion 1824—even as the plunger assembly 1822 moves through the barrel 1823—to, for example, define a sealed volume (e.g., of negative/vacuum pressure) within the barrel 1823. As described in greater detail below with reference to FIG. 18B, the plunger assembly 1822 extends into the pneumatic cylinder 1818.

The housing 1819 can further define a lumen 1835 (generally obscured in FIG. 18A and shown schematically) extending therethrough through the storage portion 1821 towards the coupling portion 1820. The lumen 1835 can be shaped to accommodate a canister containing pressurized fluid (e.g., a pressurized canister; not shown, such as a carbon dioxide canister). In some embodiments, the housing 1819 can include features configured to lock the pressurized canister into place once it has been inserted sufficiently into the lumen 1835 and to allow a user to release the pressurized canister once it has been emptied or used. The housing 1819 can also include additional internal channels configured to allow wires, tubes, lines, or varying mediums to pass through the housing 1819. In some embodiments, the housing 1819 can be ergonomically designed so that a user can place their forefinger near the coupling portion 1820 while wrapping their additional fingers along the length of the housing 1819 toward the grip portion 1821 to maintain a comfortable grip. In other embodiments, the housing 1819 is not intended to be gripped by a user during operation and can, accordingly, have other shapes. For example, the pneumatic syringe 1812 can be controlled by the control system 180 (FIG. 1). In the illustrated embodiment, and as described in greater detail below, the pneumatic cylinder 1818 is fluidly coupled to a control unit 1836 (obscured in FIG. 18A and shown schematically) housed within the housing 1819 via pressure line(s) 1845 (only one shown in FIG. 2). The control unit 1836 is coupled to a button 1838 (e.g., switch, trigger), which is located on the housing 1819, and fluidly coupled to the pressurized canister (not shown) housed within the housing 1819. In some embodiments, the button 1838 is placed so that a user can easily compress (e.g., push, activate) the button 1838 with their forefinger while maintaining their grip around the housing 1819. In other embodiments, the button 1838 or other actuator is configured to be controlled by the control system 180 (FIG. 1). The button 1838 can be configured to move between a first (e.g., compressed) and a second (e.g., released, decompressed) position. As described in further detail below, actuation of the button 1838 is configured to move the plunger assembly 1822 between a first (e.g., depressed) position and second (e.g., withdrawn) position, respectively. In other embodiments, the button 1838 can be electrically connected to the control unit 1836.

Referring to FIG. 18B, the housing 1819 (FIG. 18A) is omitted for the sake of clarity, but it should be generally understood that any number of elements of the pneumatic syringe 1810 can be housed partially or entirely within the housing 1819. In the illustrated embodiment, the pneumatic cylinder 1818 includes a barrel 1839 having a distal chamber 1840 and a proximal chamber 1841. The barrel 1839 can be coupled to and or integrally formed with the barrel 1823 of the syringe 1817 and fluidly separated therefrom by a shared seal wall 1850. The shaft 1829 of the syringe 1817 can extend through the wall 1850 and into the barrel 1839 of the pneumatic cylinder 1818. The shaft 1829 can sealingly engage an opening in the wall 1850 such that the barrel 1823 of the syringe 1817 is fluidly disconnected from the barrel 1839 of the pneumatic cylinder 1818. In the illustrated embodiment, the proximal end portion 1830 of the shaft 1829 is movably positioned within the barrel 1839 and coupled to (e.g., integrally formed, releasably or permanently attached to) a sealing head 1842. In the illustrated embodiment, the sealing head 1842 receives/holds a sealing member 1843, such as an O-ring in, for example, the same manner the sealing head 1832 of the syringe 1817 receives the sealing member 1834. The sealing head 1842 is configured to sealingly engage an interior surface of the barrel 1839—even as the plunger assembly 1822 moves through the barrel 1839. The sealing head 1842 accordingly divides the barrel 1839 into the distal (e.g., first) chamber 1840 and the proximal (e.g., second) chamber 1841.

In the illustrated embodiment, the sealing head 1832 divides the barrel 1823 of the syringe 1817 into a distal chamber 1852 and a proximal chamber 1851 (e.g., formed between the sealing head 1832 and the wall 1850). The barrel 1823 of the syringe 1817 can include one or more vents 1853 to the proximal chamber 1851 at and/or proximate to the proximal end portion 1825 that allow fluid (e.g., gas) to vent out of the proximal chamber 1851 of the barrel 1823.

The shaft 1829 of the plunger assembly 1822 couples the sealing head 1832 of the syringe 1817 to the sealing head 1842 of the pneumatic cylinder 1818 such that the sealing head 1832 of the syringe 1817 is configured to move in tandem with the sealing head 1842 of the pneumatic cylinder 1818, maintaining a constant distance apart from another. Accordingly, a location of the sealing head 1842 within the barrel 1839 of the pneumatic cylinder 1818 determines/controls a corresponding location of the sealing head 1832 within the barrel 1823 of the syringe 1817.

The barrel 1839 further includes a first port 1844a to the distal chamber 1840 and a second port 1844b to the proximal chamber 1841. The first and second ports 1844a and 1844b are fluidly coupled to a first pressure line 1845a and a second pressure line 1845b, respectively, which are fluidly coupled to a first port 1846a and a second port 1846b of the control unit 1836, respectively. The control unit 1836 further includes a first vent 1847a and a second vent 1847b, which are configured to alternate allowing fluid to flow out of the barrel 1839 as the pneumatic cylinder 218 cycles between a first and second position, which are described in further detail below. The first vent 1847a can be smaller in size (e.g., diameter, width, bore) than the second vent 1847b to allow less fluid (e.g., gas) to vent out of the first vent 1847a than the second vent 1847b. In some embodiments, the first and second vents 1847a and 1847b can be the same size. In other embodiments, the first and second vents 1847a and 1847b can be combined into a single vent. In the illustrated embodiment, the control unit 1836 further includes a third port 1846c fluidly connected to a third pressure line 1845c, which is fluidly coupled to a pressure regulator 1848. The first, second, and third pressure lines 1845a-c (collectively “pressure lines 1845”) can have a same or substantially same inner dimension and can be configured to allow high-pressure gaseous or other fluid mediums to pass quickly through them. In some embodiments, the pressure lines 1845 are omitted and the first and second ports 1844a and 1844b (collectively “barrel ports 1844”) and first, second, and third ports 1846a-c (collectively “regulator ports 1846”) are directly connected to the corresponding components described above. For example, in some embodiments, the third pressure line 1845c can be removed and the third port 1846c can be directly connected to the pressure regulator 1848.

In the illustrated embodiment, the pressure regulator 1848 is fluidly coupled to a pressurized canister 1849. The pressure regulator 1848 can be configured to regulate the pressure from the pressurized canister 1849 to ensure a constant pressure within the control unit 1836. In some embodiments, the connection between the pressure regulator 1848 and the pressurized canister 1849 can be configured to enable a user (or the control system 180 of FIG. 1) to quickly release a depleted pressurized canister 1849 and replace it with a new pressurized canister 1849. The pressurized canister 1849 can contain carbon dioxide (CO₂), another (e.g., high-energy density) gas, and/or a pressurized fluid. In some embodiments, the pressurized canister 1849 can contain nitrogen or inert gas such as argon. In still other embodiments, the pressurized canister 1849 can be replaced by a large remote tank (e.g., a tank of gas, water, or other hydraulic fluid) connected to the control unit 1836 via a tubing system. In some embodiments, the pneumatic syringe 1812 can include one or more additional pressure line(s) connecting the pressure regulator 1848 to the pressurized canister 1849. In the illustrated embodiment, the control unit 1836 is configured to (i) alternate directing the pressurized gas from the pressurized canister 1849 between the first port 1844a and the second port 1844b and (ii) to inversely alternate between allowing gas to vent out of the first vent 1847a and the second vent 1847b. The button 1838 can be configured to switch between these alternating configurations.

In operation, fluid (e.g., gas) from the pressurized canister 1849 can exert a constant pressure on the third port 1846c of the control unit 1836 via the third pressure line 1845c. The pressure regulator 1848 can regulate a pressure of the gas from the pressurized canister 1849 to ensure that the pressure at the third port 1846c, and accordingly the first port 1844a and/or the second port 1844b, does not slowly decrease as the pressurized canister 1849 depletes. The control unit 1836 can direct the pressurized gas to either the first port 1844a or the second port 1844b of the pneumatic cylinder 1818 depending on whether the button 1838 is in the first position (e.g., compressed, pushed) or the second position (e.g., decompressed, released), respectively. For example, when the button 1838 is in the first position, the control unit 1836 can direct the pressurized gas from the pressurized canister 1849 into the first port 1844a and the distal chamber 1840 via the first pressure line 1845a, filling the distal chamber 1840 with gas. The pressurized gas can push the sealing head 1842 of the pneumatic cylinder 1818 toward the proximal end chamber 1841 of the barrel 1839 and, correspondingly, pull (e.g., withdraw) the sealing head 1832 of the syringe 1817 proximally through the barrel 1823 of the syringe 1817 toward the proximal end portion 1825 of the barrel 1823 and the pneumatic cylinder 1818. Simultaneously, ambient gas (e.g., air) between the sealing head 1832 and the wall 1850 can vent out of the barrel 1823 to atmosphere. In effect, the vents 1853 inhibit or even prevent pressure from building up within the distal chamber 1852 of the barrel 1823 as the plunger assembly 1822 moves proximally, which could otherwise create resistance against the movement of the plunger assembly 1822. In some aspects of the present technology, the distal chamber 1840 can be rapidly pressurized in this manner to cause rapid movement of the plunger assembly 1822, nearly instantaneously generating a vacuum within the barrel 1823 of the syringe 1817. For example, the pneumatic force of the pressurized fluid can drive the plunger assembly 1822 to fully withdraw the sealing head 1832 of the syringe 1817 through the barrel 1823 from a depressed position to a withdrawn position in less than about 2 seconds, less than about 1 second, less than about 0.9 second, less than about 0.8 second, less than about 0.7 second, less than about 0.6 second, less than about 0.6 second, less than about 0.4 second, less than about 0.3 second, less than about 0.2 second, less than about 0.1 second, or less.

When the button 1838 is in a second position (e.g., decompressed, released), the control unit 1836 can direct the pressurized gas from the pressurized canister 1849 into the second port 1844b and the proximal chamber 1841 via the second pressure line 1845b, filling the proximal chamber 1841 with pressurized gas. Simultaneously, the control unit 1836 can connect the first port 1844a with the first vent 1847a via the first pressure line 1845a, allowing the pressurized gas within the distal chamber 1840 to vent to atmosphere. The pressurized gas within the proximal chamber 1841 can push/drive the sealing head 1842 of the pneumatic cylinder 1818 distally through the barrel 1839 toward the distal end chamber 1840 of the barrel 1839 and, correspondingly, the sealing head 1832 of the syringe 1817 toward the distal tip 1826 (e.g., depressing the sealing head 1832 within the barrel 1823 of the syringe 1817). The distal movement of the sealing head 1832 within the barrel 1823 can generate positive pressure in the distal chamber 1852 of the syringe 1817 to, for example, drive any material aspirated into the syringe 1817 out of the distal tip 1826. The vents 1853 allow ambient gas to fill the proximal chamber 1851 to inhibit or even prevent vacuum from forming in the proximal chamber 1851 during distal movement of the sealing head 1832. If the button 1838 is pressed again (e.g., moved from the second position to the first position), the control unit 1836 can again direct the pressurized gas from the pressurized canister 1849 into the first port 1844a via the first pressure line 1845a and connect the second port 1844b with the second vent 1847b via the second pressure line 1845b, to drive the plunger assembly 1822 to retract/withdraw proximally as described in detail above.

### V. SELECTED EXAMPLES

The following examples are illustrative of several embodiments of the present technology:

1. A system for treating clot material in a vasculature of a patient, comprising:
an aspiration catheter defining an aspiration lumen and having a distal end portion, wherein the aspiration catheter is configured to be positioned within the vasculature of the patient such that the distal end portion is positioned proximate to the clot material;
a reinfusion catheter defining a reinfusion lumen, wherein the reinfusion catheter is configured to be positioned within the vasculature of the patient;
an aspiration assembly comprising—
   an aspiration inlet valve;
   an aspiration outlet valve; and
   an aspiration syringe configured to be fluidly coupled to the aspiration lumen of the aspiration catheter via the aspiration valve;
a filter assembly comprising—
   a filter inlet configured to be fluidly coupled to the aspiration syringe via the aspiration outlet valve;
   a filter outlet; and
   a filter between the filter inlet and the filter outlet;
a reinfusion assembly comprising—
   a reinfusion valve; and
   a reinfusion syringe configured to be fluidly coupled to (a) the filter outlet and (b) the reinfusion lumen of the reinfusion catheter via the reinfusion valve; and
a control assembly communicatively coupled to the aspiration assembly and the reinfusion assembly, wherein the control assembly includes a non-transitory computer-readable storage medium storing instructions that, when executed by the control assembly, cause (a) the aspiration assembly to selectively generate vacuum pressure and positive pressure in the aspiration syringe and (b) the reinfusion assembly to selectively generate vacuum pressure and positive pressure in the reinfusion syringe.

2. The system of example 1 wherein the instructions, when executed by the control assembly, further cause:
actuation of the aspiration syringe to generate the vacuum pressure in the aspiration syringe to aspirate at least a portion of the clot material and blood through the aspiration lumen of the aspiration catheter and the aspiration inlet valve into the aspiration syringe;
actuation of the aspiration syringe to generate the positive pressure in the aspiration syringe to drive the portion of the clot material and the blood from the aspiration syringe through the aspiration outlet valve into the filter assembly, wherein the filter is configured to filter the portion of the clot material from the blood;
actuation of the reinfusion syringe to generate the vacuum pressure in the reinfusion syringe to draw the filtered blood through the filter outlet and into the aspiration syringe; and
actuation of the reinfusion syringe to generate the positive pressure in the reinfusion syringe to drive the filtered blood through the reinfusion valve and into the reinfusion lumen for reinfusion into the vasculature of the patient.

3. The system of example 2 wherein the instructions, when executed by the control assembly, further cause:
closure of the aspiration inlet valve;
actuation of the aspiration syringe to generate the vacuum pressure in the aspiration syringe with the aspiration inlet valve closed to generate stored vacuum pressure in the aspiration syringe; and
opening of the aspiration inlet valve to apply the stored vacuum pressure to the aspiration lumen of the aspiration catheter to aspirate the portion of the clot material and the blood through the aspiration lumen of the aspiration catheter and the aspiration inlet valve and into the aspiration syringe.

4. The system of example 3 wherein the aspiration inlet valve comprises a stopcock.

5. The system of any one of examples 1-4 wherein the aspiration syringe comprises a plunger slidably positioned within a barrel and a pneumatic actuator configured to drive the plunger through the barrel in a first direction and a second direction through the barrel.

6. The system of example 5 wherein the instructions, when executed by the control assembly, further cause the pneumatic actuator to (a) withdraw the plunger through the barrel in the first direction to generate the vacuum pressure in the aspiration syringe and (b) depress the plunger through the barrel in the second direction to generate the positive pressure.

7. The system of any one of examples 1-6 wherein the aspiration syringe comprises a plunger slidably positioned within a barrel and an electromechanical actuator configured to drive the plunger through the barrel in a first direction and a second direction through the barrel.

8. The system of example 7 wherein the instructions, when executed by the control assembly, further cause the electromechanical actuator to (a) withdraw the plunger through the barrel in the first direction to generate the vacuum pressure in the aspiration syringe and (b) depress the plunger through the barrel in the second direction to generate the positive pressure.

9. The system of any one of examples 1-8 wherein the aspiration inlet valve comprises a one-way valve configured to (a) permit fluid flow from the aspiration lumen to the aspiration syringe and (b) inhibit fluid flow from the aspiration syringe to the aspiration lumen.

10. The system of any one of examples 1-9 wherein—
the aspiration inlet valve comprises an electromechanical valve; and
the instructions, when executed by the control assembly, further cause the electromechanical valve to (a) open to permit fluid flow from the aspiration lumen to the aspiration syringe and (b) close to inhibit fluid flow from the aspiration syringe to the aspiration lumen.

11. The system of any one of examples 1-10 wherein the aspiration catheter and the reinfusion catheter comprise the same catheter, and wherein the aspiration lumen extends at least partially parallel to and separate from the reinfusion lumen through the reinfusion catheter.

12. The system of any one of examples 1-11 wherein the aspiration catheter is separate and spaced apart from the reinfusion catheter.

13. A system for treating clot material in a vasculature of a patient, comprising:
an aspiration catheter defining an aspiration lumen and having a distal end portion, wherein the aspiration catheter is configured to be positioned within the vasculature of the patient such that the distal end portion is positioned proximate to the clot material;
a reinfusion catheter defining a reinfusion lumen, wherein the reinfusion catheter is configured to be positioned within the vasculature of the patient;
an aspiration assembly comprising—
   an aspiration inlet tube fluidly coupled to the aspiration lumen of the aspiration catheter;
   an aspiration outlet tube;
   aspiration connection tubing;
   an aspiration syringe having a plunger slidable within a barrel and an aspiration syringe actuator configured to slide the plunger through the barrel;
   an aspiration inlet valve positioned between the aspiration connection tubing and the aspiration inlet tube;
   an aspiration syringe valve positioned between the aspiration connection tubing and the aspiration syringe; and
   an aspiration outlet valve positioned between the aspiration connection tubing and the aspiration outlet tube;
a filter assembly comprising—
   a filter inlet fluidly coupled to the aspiration outlet tube;
   a filter conduit;
   a filter between the filter inlet and the filter conduit; and
   a filter valve;
a reinfusion assembly comprising—
   reinfusion connection tubing fluidly coupled to the filter conduit via the filter valve;
   a reinfusion outlet tube fluidly coupled to the reinfusion lumen of the reinfusion catheter;
   a reinfusion syringe having a plunger slidable within a barrel and a reinfusion syringe actuator configured to slide the plunger through the barrel;
   a reinfusion outlet valve positioned between the reinfusion connection tubing and the reinfusion outlet tube; and
   a reinfusion syringe valve positioned between the reinfusion connection tubing and the reinfusion syringe; and
a processing device communicatively coupled to the aspiration assembly, the filter assembly, and the reinfusion assembly.

14. The system of example 13 wherein the processing device is configured to perform a sequence comprising—
closing each of the aspiration inlet valve, the aspiration syringe valve, the aspiration outlet valve, the filter valve, the reinfusion outlet valve, and the reinfusion syringe valve;
opening the aspiration inlet valve;
actuating the aspiration syringe actuator to withdraw the plunger of the aspiration syringe valve to generate vacuum pressure within the barrel of the aspiration syringe;
opening the aspiration syringe valve to apply the vacuum pressure to the aspiration lumen of the aspiration catheter to aspirate at least a portion of the clot material and blood through the aspiration lumen into the barrel of the aspiration syringe;
closing the aspiration inlet valve;
opening the aspiration outlet valve;
actuating the aspiration syringe actuator to depress the plunger of the aspiration syringe to drive the portion of the clot material and the blood from the barrel of the aspiration syringe into the filter assembly through the filter inlet;
opening the filter valve;
opening the reinfusion syringe valve;
actuating the reinfusion syringe actuator to withdraw the plunger of the reinfusion syringe valve to draw the blood through the filter into the barrel of the reinfusion syringe, wherein the filter is configured to inhibit the portion of the clot material from passing through the filter to the barrel of the reinfusion syringe;
closing the filter valve;
opening the reinfusion outlet valve; and
actuating the reinfusion syringe actuator to depress the plunger of the reinfusion syringe valve to drive the blood through from the barrel of the reinfusion syringe into the reinfusion lumen for reinfusion into the vasculature of the patient.

15. The system of example 13 or example 14, further comprising:
a flushing syringe having a plunger slidable within a barrel and a flushing syringe actuator configured to slide the plunger through the barrel, and wherein the barrel of the flushing syringe is configured to hold a flushing fluid; and
a flushing valve positioned between the reinfusion connection tubing and the flushing syringe.

16. The system of example 15 wherein the processing device is configured to perform a sequence comprising—
closing each of the aspiration inlet valve, the aspiration syringe valve, the aspiration outlet valve, the filter valve, the reinfusion outlet valve, and the reinfusion syringe valve;
opening the aspiration inlet valve;
actuating the aspiration syringe actuator to withdraw the plunger of the aspiration syringe valve to generate vacuum pressure within the barrel of the aspiration syringe;
opening the aspiration syringe valve to apply the vacuum pressure to the aspiration lumen of the aspiration catheter to aspirate at least a portion of the clot material and the blood through the aspiration lumen into the barrel of the aspiration syringe;
closing the aspiration inlet valve;
opening the aspiration outlet valve;
actuating the aspiration syringe actuator to depress the plunger of the aspiration syringe to drive the portion of the clot material and the blood from the barrel of the aspiration syringe into the filter assembly through the filter inlet;
opening the filter valve;
opening the reinfusion syringe valve;
actuating the reinfusion syringe actuator to withdraw the plunger of the reinfusion syringe valve to draw the blood through the filter into the barrel of the reinfusion syringe, wherein the filter is configured to inhibit the portion of the clot material from passing through the filter to the barrel of the reinfusion syringe;
closing the filter valve;
opening the reinfusion outlet valve;
actuating the reinfusion syringe actuator to depress the plunger of the reinfusion syringe valve to drive the blood through from the barrel of the reinfusion syringe into the reinfusion lumen for reinfusion into the vasculature of the patient;
closing the reinfusion syringe valve;
opening the flushing valve; and
actuating the flushing syringe actuator to at least partially depress the plunger of the flushing syringe to drive the flushing fluid from the barrel of the flushing syringe into the reinfusion lumen to push the blood through the reinfusion lumen.

17. The system of example 15 or example 16 wherein the processing device is configured to perform a sequence comprising—
closing each of the aspiration inlet valve, the aspiration syringe valve, the aspiration outlet valve, the filter valve, the reinfusion outlet valve, and the reinfusion syringe valve;
opening the aspiration inlet valve;
actuating the aspiration syringe actuator to withdraw the plunger of the aspiration syringe valve to generate vacuum pressure within the barrel of the aspiration syringe;
opening the aspiration syringe valve to apply the vacuum pressure to the aspiration lumen of the aspiration catheter to aspirate at least a portion of the clot material and the blood through the aspiration lumen into the barrel of the aspiration syringe;
closing the aspiration inlet valve;
opening the aspiration outlet valve;
actuating the aspiration syringe actuator to depress the plunger of the aspiration syringe to drive the portion of the clot material and the blood from the barrel of the aspiration syringe into the filter assembly through the filter inlet;
opening the filter valve;
opening the reinfusion syringe valve;
actuating the reinfusion syringe actuator to withdraw the plunger of the reinfusion syringe valve to draw the blood through the filter into the barrel of the reinfusion syringe, wherein the filter is configured to inhibit the portion of the clot material from passing through the filter to the barrel of the reinfusion syringe;
closing the filter valve;
opening the reinfusion outlet valve;
actuating the reinfusion syringe actuator to depress the plunger of the reinfusion syringe valve to drive the blood through from the barrel of the reinfusion syringe into the reinfusion lumen for reinfusion into the vasculature of the patient;
closing the reinfusion syringe valve;
closing the reinfusion outlet valve;
opening the flushing syringe valve;
opening the filter valve; and
actuating the flushing syringe actuator to at least partially depress the plunger of the flushing syringe to drive the flushing fluid from the barrel of the flushing syringe into the filter assembly to flush the filter with the flushing fluid.

18. The system of any one of examples 13-17 wherein the aspiration syringe is a first aspiration syringe, wherein the aspiration syringe actuator is a first aspiration syringe actuator, wherein the aspiration syringe valve is a first aspiration syringe valve, and wherein the aspiration assembly further comprises:
a second aspiration syringe having a plunger slidable within a barrel and a second aspiration syringe actuator configured to slide the plunger through the barrel; and
a second aspiration syringe valve positioned between the aspiration connection tubing and the second aspiration syringe.

19. The system of any one of examples 13-18 wherein the aspiration syringe actuator comprises a pneumatic actuator.

20. The system of any one of examples 13-19 wherein the aspiration syringe actuator comprises an electric motor.

21. A system for treating clot material in a vasculature of a patient, comprising:
an aspiration catheter defining an aspiration lumen and having a distal end portion, wherein the aspiration catheter is configured to be positioned within the vasculature of the patient such that the distal end portion is positioned proximate to the clot material;
a reinfusion catheter defining a reinfusion lumen, wherein the reinfusion catheter is configured to be positioned within the vasculature of the patient;
an aspiration assembly comprising—
   an aspiration inlet tube fluidly coupled to the aspiration lumen of the aspiration catheter;
   an aspiration outlet tube;
   aspiration connection tubing;
   an aspiration syringe having a plunger slidable within a barrel and an aspiration syringe actuator configured to slide the plunger through the barrel;
   an aspiration inlet valve positioned between the aspiration connection tubing and the aspiration inlet tube;
   an aspiration syringe valve positioned between the aspiration connection tubing and the aspiration syringe; and
   an aspiration outlet valve positioned between the aspiration connection tubing and the aspiration outlet tube;
a filter assembly comprising—
   a filter inlet fluidly coupled to the aspiration outlet tube;
   a filter conduit;
   a filter between the filter inlet and the filter conduit; and
   a filter valve;
a reinfusion assembly comprising—reinfusion connection tubing fluidly coupled to the filter conduit via the filter valve;
   a reinfusion outlet tube fluidly coupled to the reinfusion lumen of the reinfusion catheter;
   a reinfusion syringe having a plunger slidable within a barrel and a reinfusion syringe actuator configured to slide the plunger through the barrel;
   a reinfusion outlet valve positioned between the reinfusion connection tubing and the reinfusion outlet tube; and
   a reinfusion syringe valve positioned between the reinfusion connection tubing and the reinfusion syringe.

### VI. CONCLUSION

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for treating clot material in a vasculature of a patient, comprising:
an aspiration catheter defining an aspiration lumen and having a distal end portion, wherein the aspiration catheter is configured to be positioned within the vasculature of the patient such that the distal end portion is positioned proximate to the clot material;
a reinfusion catheter defining a reinfusion lumen, wherein the reinfusion catheter is configured to be positioned within the vasculature of the patient;
an aspiration assembly comprising—
an aspiration inlet valve;
an aspiration outlet valve; and
an aspiration syringe configured to be fluidly coupled to the aspiration lumen of the aspiration catheter via the aspiration valve;
a filter assembly comprising—
a filter inlet configured to be fluidly coupled to the aspiration syringe via the aspiration outlet valve;
a filter outlet; and
a filter between the filter inlet and the filter outlet;
a reinfusion assembly comprising—
a reinfusion valve; and
a reinfusion syringe configured to be fluidly coupled to (a) the filter outlet and (b) the reinfusion lumen of the reinfusion catheter via the reinfusion valve; and
a control assembly communicatively coupled to the aspiration assembly and the reinfusion assembly, wherein the control assembly includes a non-transitory computer-readable storage medium storing instructions that, when executed by the control assembly, cause (a) the aspiration assembly to selectively generate vacuum pressure and positive pressure in the aspiration syringe and (b) the reinfusion assembly to selectively generate vacuum pressure and positive pressure in the reinfusion syringe.

2. The system of claim 1 wherein the instructions, when executed by the control assembly, further cause:
actuation of the aspiration syringe to generate the vacuum pressure in the aspiration syringe to aspirate at least a portion of the clot material and blood through the aspiration lumen of the aspiration catheter and the aspiration inlet valve into the aspiration syringe;
actuation of the aspiration syringe to generate the positive pressure in the aspiration syringe to drive the portion of the clot material and the blood from the aspiration syringe through the aspiration outlet valve into the filter assembly, wherein the filter is configured to filter the portion of the clot material from the blood;
actuation of the reinfusion syringe to generate the vacuum pressure in the reinfusion syringe to draw the filtered blood through the filter outlet and into the aspiration syringe; and
actuation of the reinfusion syringe to generate the positive pressure in the reinfusion syringe to drive the filtered blood through the reinfusion valve and into the reinfusion lumen for reinfusion into the vasculature of the patient.

3. The system of claim 2 wherein the instructions, when executed by the control assembly, further cause:
closure of the aspiration inlet valve;
actuation of the aspiration syringe to generate the vacuum pressure in the aspiration syringe with the aspiration inlet valve closed to generate stored vacuum pressure in the aspiration syringe; and
opening of the aspiration inlet valve to apply the stored vacuum pressure to the aspiration lumen of the aspiration catheter to aspirate the portion of the clot material and the blood through the aspiration lumen of the aspiration catheter and the aspiration inlet valve and into the aspiration syringe.

4. The system of claim 3 wherein the aspiration inlet valve comprises a stopcock.

5. The system of claim 1 wherein the aspiration syringe comprises a plunger slidably positioned within a barrel and a pneumatic actuator configured to drive the plunger through the barrel in a first direction and a second direction through the barrel.

6. The system of claim 5 wherein the instructions, when executed by the control assembly, further cause the pneumatic actuator to (a) withdraw the plunger through the barrel in the first direction to generate the vacuum pressure in the aspiration syringe and (b) depress the plunger through the barrel in the second direction to generate the positive pressure.

7. The system of claim 1 wherein the aspiration syringe comprises a plunger slidably positioned within a barrel and an electromechanical actuator configured to drive the plunger through the barrel in a first direction and a second direction through the barrel.

8. The system of claim 7 wherein the instructions, when executed by the control assembly, further cause the electromechanical actuator to (a) withdraw the plunger through the barrel in the first direction to generate the vacuum pressure in the aspiration syringe and (b) depress the plunger through the barrel in the second direction to generate the positive pressure.

9. The system of claim 1 wherein the aspiration inlet valve comprises a one-way valve configured to (a) permit fluid flow from the aspiration lumen to the aspiration syringe and (b) inhibit fluid flow from the aspiration syringe to the aspiration lumen.

10. The system of claim 1 wherein—
the aspiration inlet valve comprises an electromechanical valve; and
the instructions, when executed by the control assembly, further cause the electromechanical valve to (a) open to permit fluid flow from the aspiration lumen to the aspiration syringe and (b) close to inhibit fluid flow from the aspiration syringe to the aspiration lumen.

11. The system of claim 1 wherein the aspiration catheter and the reinfusion catheter comprise the same catheter, and wherein the aspiration lumen extends at least partially parallel to and separate from the reinfusion lumen through the reinfusion catheter.

12. The system of claim 1 wherein the aspiration catheter is separate and spaced apart from the reinfusion catheter.

13. A system for treating clot material in a vasculature of a patient, comprising:
an aspiration catheter defining an aspiration lumen and having a distal end portion, wherein the aspiration catheter is configured to be positioned within the vasculature of the patient such that the distal end portion is positioned proximate to the clot material;
a reinfusion catheter defining a reinfusion lumen, wherein the reinfusion catheter is configured to be positioned within the vasculature of the patient;
an aspiration assembly comprising—
an aspiration inlet tube fluidly coupled to the aspiration lumen of the aspiration catheter;
an aspiration outlet tube;
aspiration connection tubing;
an aspiration syringe having a plunger slidable within a barrel and an aspiration syringe actuator configured to slide the plunger through the barrel;
an aspiration inlet valve positioned between the aspiration connection tubing and the aspiration inlet tube;
an aspiration syringe valve positioned between the aspiration connection tubing and the aspiration syringe; and
an aspiration outlet valve positioned between the aspiration connection tubing and the aspiration outlet tube;
a filter assembly comprising—
a filter inlet fluidly coupled to the aspiration outlet tube;
a filter conduit;
a filter between the filter inlet and the filter conduit; and
a filter valve;
a reinfusion assembly comprising—
reinfusion connection tubing fluidly coupled to the filter conduit via the filter valve;
a reinfusion outlet tube fluidly coupled to the reinfusion lumen of the reinfusion catheter;
a reinfusion syringe having a plunger slidable within a barrel and a reinfusion syringe actuator configured to slide the plunger through the barrel;
a reinfusion outlet valve positioned between the reinfusion connection tubing and the reinfusion outlet tube; and
a reinfusion syringe valve positioned between the reinfusion connection tubing and the reinfusion syringe; and
a processing device communicatively coupled to the aspiration assembly, the filter assembly, and the reinfusion assembly.

14. The system of claim 13 wherein the processing device is configured to perform a sequence comprising—
closing each of the aspiration inlet valve, the aspiration syringe valve, the aspiration outlet valve, the filter valve, the reinfusion outlet valve, and the reinfusion syringe valve;
opening the aspiration inlet valve;
actuating the aspiration syringe actuator to withdraw the plunger of the aspiration syringe valve to generate vacuum pressure within the barrel of the aspiration syringe;
opening the aspiration syringe valve to apply the vacuum pressure to the aspiration lumen of the aspiration catheter to aspirate at least a portion of the clot material and blood through the aspiration lumen into the barrel of the aspiration syringe;
closing the aspiration inlet valve;
opening the aspiration outlet valve;
actuating the aspiration syringe actuator to depress the plunger of the aspiration syringe to drive the portion of the clot material and the blood from the barrel of the aspiration syringe into the filter assembly through the filter inlet;
opening the filter valve;
opening the reinfusion syringe valve;
actuating the reinfusion syringe actuator to withdraw the plunger of the reinfusion syringe valve to draw the blood through the filter into the barrel of the reinfusion syringe, wherein the filter is configured to inhibit the portion of the clot material from passing through the filter to the barrel of the reinfusion syringe;
closing the filter valve;
opening the reinfusion outlet valve; and
actuating the reinfusion syringe actuator to depress the plunger of the reinfusion syringe valve to drive the blood through from the barrel of the reinfusion syringe into the reinfusion lumen for reinfusion into the vasculature of the patient.

15. The system of claim 13, further comprising:
a flushing syringe having a plunger slidable within a barrel and a flushing syringe actuator configured to slide the plunger through the barrel, and wherein the barrel of the flushing syringe is configured to hold a flushing fluid; and
a flushing valve positioned between the reinfusion connection tubing and the flushing syringe.

16. The system of claim 15 wherein the processing device is configured to perform a sequence comprising—
closing each of the aspiration inlet valve, the aspiration syringe valve, the aspiration outlet valve, the filter valve, the reinfusion outlet valve, and the reinfusion syringe valve;
opening the aspiration inlet valve;
actuating the aspiration syringe actuator to withdraw the plunger of the aspiration syringe valve to generate vacuum pressure within the barrel of the aspiration syringe;
opening the aspiration syringe valve to apply the vacuum pressure to the aspiration lumen of the aspiration catheter to aspirate at least a portion of the clot material and the blood through the aspiration lumen into the barrel of the aspiration syringe;
closing the aspiration inlet valve;
opening the aspiration outlet valve;
actuating the aspiration syringe actuator to depress the plunger of the aspiration syringe to drive the portion of the clot material and the blood from the barrel of the aspiration syringe into the filter assembly through the filter inlet;
opening the filter valve;
opening the reinfusion syringe valve;
actuating the reinfusion syringe actuator to withdraw the plunger of the reinfusion syringe valve to draw the blood through the filter into the barrel of the reinfusion syringe, wherein the filter is configured to inhibit the portion of the clot material from passing through the filter to the barrel of the reinfusion syringe;
closing the filter valve;
opening the reinfusion outlet valve;
actuating the reinfusion syringe actuator to depress the plunger of the reinfusion syringe valve to drive the blood through from the barrel of the reinfusion syringe into the reinfusion lumen for reinfusion into the vasculature of the patient;
closing the reinfusion syringe valve;
opening the flushing valve; and
actuating the flushing syringe actuator to at least partially depress the plunger of the flushing syringe to drive the flushing fluid from the barrel of the flushing syringe into the reinfusion lumen to push the blood through the reinfusion lumen.

17. The system of claim 15 wherein the processing device is configured to perform a sequence comprising—
closing each of the aspiration inlet valve, the aspiration syringe valve, the aspiration outlet valve, the filter valve, the reinfusion outlet valve, and the reinfusion syringe valve;
opening the aspiration inlet valve;
actuating the aspiration syringe actuator to withdraw the plunger of the aspiration syringe valve to generate vacuum pressure within the barrel of the aspiration syringe;
opening the aspiration syringe valve to apply the vacuum pressure to the aspiration lumen of the aspiration catheter to aspirate at least a portion of the clot material and the blood through the aspiration lumen into the barrel of the aspiration syringe;
closing the aspiration inlet valve;
opening the aspiration outlet valve;
actuating the aspiration syringe actuator to depress the plunger of the aspiration syringe to drive the portion of the clot material and the blood from the barrel of the aspiration syringe into the filter assembly through the filter inlet;
opening the filter valve;
opening the reinfusion syringe valve;
actuating the reinfusion syringe actuator to withdraw the plunger of the reinfusion syringe valve to draw the blood through the filter into the barrel of the reinfusion syringe, wherein the filter is configured to inhibit the portion of the clot material from passing through the filter to the barrel of the reinfusion syringe;
closing the filter valve;
opening the reinfusion outlet valve;
actuating the reinfusion syringe actuator to depress the plunger of the reinfusion syringe valve to drive the blood through from the barrel of the reinfusion syringe into the reinfusion lumen for reinfusion into the vasculature of the patient;
closing the reinfusion syringe valve;
closing the reinfusion outlet valve;
opening the flushing syringe valve;
opening the filter valve; and
actuating the flushing syringe actuator to at least partially depress the plunger of the flushing syringe to drive the flushing fluid from the barrel of the flushing syringe into the filter assembly to flush the filter with the flushing fluid.

18. The system of claim 13 wherein the aspiration syringe is a first aspiration syringe, wherein the aspiration syringe actuator is a first aspiration syringe actuator, wherein the aspiration syringe valve is a first aspiration syringe valve, and wherein the aspiration assembly further comprises:
a second aspiration syringe having a plunger slidable within a barrel and a second aspiration syringe actuator configured to slide the plunger through the barrel; and
a second aspiration syringe valve positioned between the aspiration connection tubing and the second aspiration syringe.

19. The system of claim 13 wherein the aspiration syringe actuator comprises a pneumatic actuator.

20. The system of claim 13 wherein the aspiration syringe actuator comprises an electric motor.
